(19) United States
(12) Patent Application Publication
(10) Pub. No.: US 2008/0039625 A1
(43) Pub. Date: Feb. 14, 2008

(54) Screening Methods

(76) Inventors: Mark Lautens, Etobicoke (CA); Yuanqing Fang, Somerville, MA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/718,769

(22) PCT Filed: Nov. 4, 2005
(86) PCT No.: PCT/CA05/01703

§ 371 Date: Sep. 24, 2007

Related U.S. Application Data
(60) Provisional application No. 60/625,102, filed on Nov. 5, 2004.
(60) Provisional application No. 60/662,797, filed on Mar. 18, 2005.

(51) Int. Cl. C07D 209/08; C07D 209/10; C07D 401/14

(52) U.S. Cl. 544/363; 548/509; 548/510; 548/511

(57) ABSTRACT

Disclosed are processes for the preparation of 2-substituted indole compounds wherein the 2-substituent comprises an R₄ group, wherein R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond; the process comprising reacting an orthogem-dihalovinylaniline compound of the formula (I): wherein Halo comprises Br, Cl, or I; each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the indole ring; all of which are optionally substituted with one or more suitable substituents at one or more substitutable positions; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents; R₃ comprises H, alkyl, haloalkyl, alkenyl, alkynyl, aryl, heteroaryl, cycloalkyl, heterocycle, aryl-(C₁₋₆)alkyl-, or heteroaryl-loweralkyl-, all of which are optionally substituted at one or more substitutable positions with one or more suitable substituents; with an organoboron reagent selected from the group consisting of a boronic ester of R₄, a boronic acid of R₄, a boronic acid anhydride of R₄, a trialkylborane of R₄ and a 9-BBN derivative of R₄; in the presence of a base, a palladium metal pre-catalyst and a ligand under reaction conditions effective to form the 2-substituted indole compound. Also disclosed are processes for the preparation of ortho-gem-dihalovinylaniline compounds. Novel compounds prepared by the processes and novel uses of the compounds are likewise disclosed.

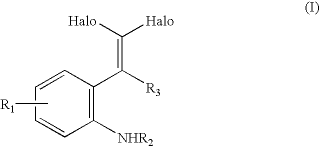

### BACKGROUND OF THE INVENTION

1. Field of the Invention

The present invention relates generally to processes for the chemical synthesis of indole compounds, in particular indole compounds that are substituted at the 2-position of the indole ring, and optionally at additional locations of the indole ring such as the 1- and/or 3-position, compounds prepared by such processes, and synthetic precursors of such processes. More particularly, the present invention relates to the preparation of 2-substituted indole compounds from an ortho-gem-dihalovinylaniline compound and an organoboron reagent using a palladium pre-catalyst, base and a ligand. The present invention also relates to processes for the production of ortho-gem-dibromovinylanilines which are useful as starting materials in the production of 2-substituted indoles, and novel compounds prepared by the processes.

2. Brief Description of the Related Art

The indole moiety is a privileged structural motif exhibiting pharmacological properties in numerous therapeutic agents and natural products (for example, see Somei, M.; Yamada, F. Nat. Prod. Rep. 2004, 21, 278-311; Somei, M.; Yamada, F. Nat. Prod. Rep. 2003, 20, 216-242. (c) Somei, M. Adv. Heterocycl. Chem. 2002, 82, 101-155). A brief survey of the scientific literature demonstrates the ubiquitous nature of indoles, as numerous drugs currently on the market contain the indole structure either in the final pharmaceutical agent as a substructure or as intermediate compound en route to the final target molecule. Consequently, methodology giving access to new indole derivatives is attractive to many synthetic and medicinal chemists (see Gribble, G. W. Perkin 1 2000, 1045-1075; Sundberg, R. J.; Editor Indoles, Academic Press, San Diego, Calif., 1996). In particular, modular synthetic methods are desirable due to their ability to rapidly synthesize a library of indole derivatives using traditional combinatorial approaches (Horton, D. A.; Bourne, G. T.; Smythe, M. L. Chem. Rev. 2003, 103, 893-930; Thompson, L. A.; Ellman, J. A. Chem. Rev. 1996, 96, 555-600).

Previous work in the field has lead to the development of numerous processes for the synthesis of indoles and derivatives thereof, several of which are shown below with the reported yields for the preparation of various indoles.

To date, many of the prior art processes are reported to have numerous drawbacks such as being inefficient, requiring multiple steps, requiring commercially unavailable or expensive starting materials, requiring the use of harsh reaction conditions, and/or are challenging to adapt to an industrial scale. A general description of several prior art processes is set out below in Schemes 1-23, additional details of which are set out in the references as indicated.

Fisher indole synthesis (Scheme 1) is one of the most commonly used methods for indole synthesis (Robinson, B. The Fischer Indole Synthesis, 1982). However, for some cases, yields may be low. The reaction can be done either in one pot or via isolation of the hydrazone. Relatively harsh conditions are called for as Lewis acids are normally required as a catalyst and reactions are typically carried out at high temperature. When the starting hydrazine is meta-substituted, two possible isomeric products can be produced as a mixture. Electron-poor hydrazines are normally retarded starting materials and 4-substituted and 2-alkyl substituted indoles have been reported to be particularly challenging to make via this method.

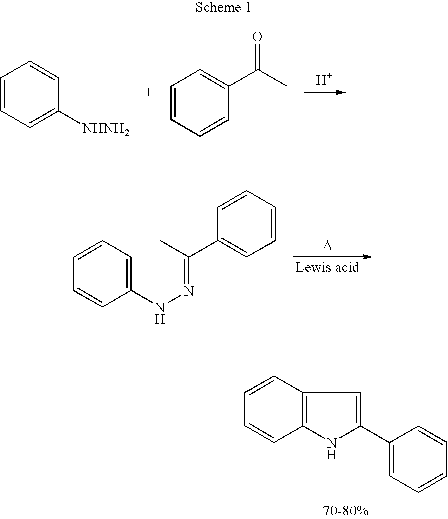

As shown in Scheme 2, Buchwald and coworkers have developed Pd-catalyzed a C—N coupling reaction between diphenylhydrazone and aryl bromide to form a hydrazone intermediate and applied Fischer indoles synthesis methodology to make functionalized indoles (Wagaw, S.; Yang, B. H.; Buchwald, S. L. J. Am. Chem. Soc. 1998, 120, 6621-6622).

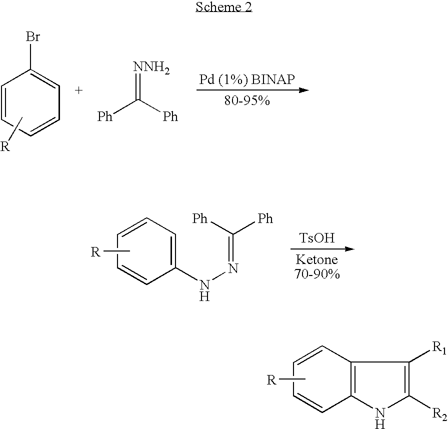

Another example, known as the classic Reissert indole synthesis is also a common method (Scheme 3). It involves reductive cyclization of o-nitrophenylpyruvic acid to give indole-2-carboxylate (Noland, W. E.; Baude, F. J. Org. Synth. 1963, 43, 40-45).

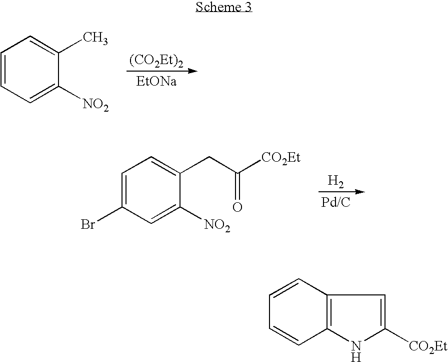

A modified method was reported by Clark and coworkers (see Scheme 4) to make functionalized indoles was further developed for which yields have been reported to be generally low (Clark, C. I.; White, J. M.; Kelly, D. P.; Martin, R. F.; Lobachevsky, P. Aust. J. Chem. 1998, 51, 243-247).

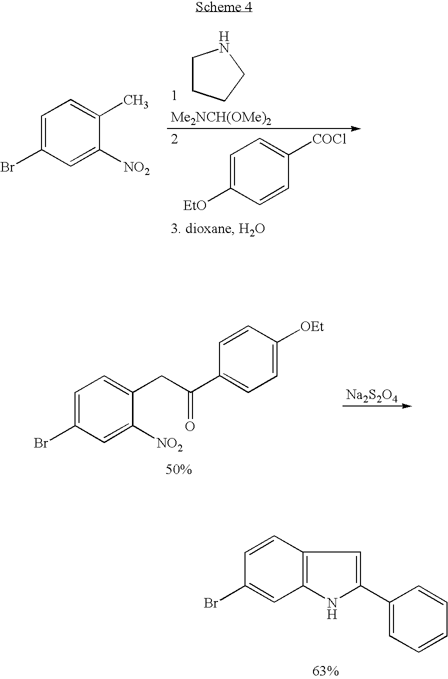

In another example, Buchwald also developed Pd-catalyzed coupling between o-halonitrobenzene and methyl ketone to give an intermediate which was reductively cyclized to give highly substituted indoles (see Scheme 5) (Rutherford, J. L.; Rainka, M. P.; Buchwald, S. L. J. Am. Chem. Soc. 2002, 124, 15168-15169).

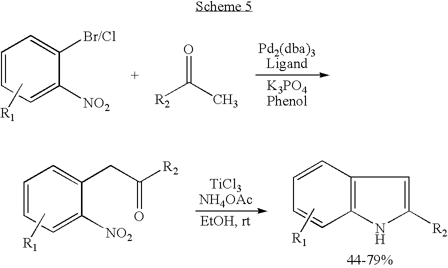

In yet another example in Scheme 6, Madelung indole synthesis uses o-methylacetanilide as a starting material and a strong base such as NaNH₂ or n-BuLi (Houlihan, W. J.; Parrino, V. A.; Uike, Y. J. Org. Chem. 1981, 46, 4511-4515).

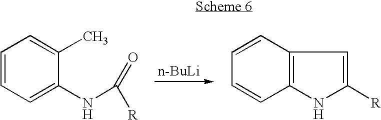

In yet another example, 2-nitrostyrene has been reported as a precursor for preparing substituted indoles via reductive cyclization methodologies (Scheme 7). The reducing agent can be CO/Pd (Soederberg, B. C.; Shriver, J. A.; Wallace, J. M. Org. Synth. 2003, 80, 75-84) or CO/Se system (Nishiyama, Y.; Maema, R.; Ohno, K.; Hirose, M.; Sonoda, N. Tetrahedron Lett. 1999, 40, 5717-5720). Relatively high pressures of CO and high catalyst loading (6%) are reported to have been used.

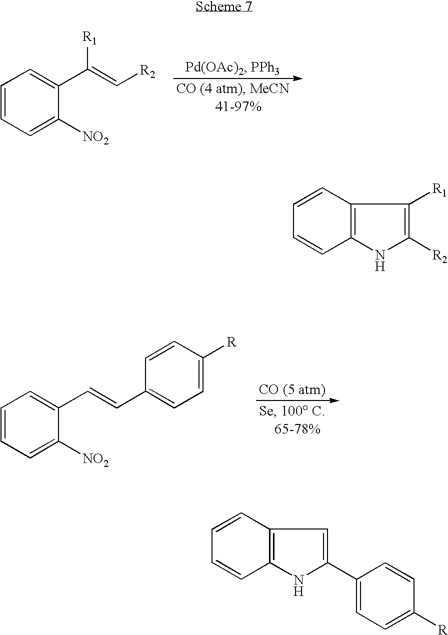

2-substituted indoles can also be made from o-azastyrenes using the Sundberg indole synthesis (Scheme 8). High temperature and instability of azides may make this method less favoured for industrial process (Molina, P.; Alcantara, J.; Lopez-Leonardo, C. Tetrahedron Lett. 1995, 36, 953-956; Molina, P.; Alcantara, J.; Lopez-Leonardo, C. Tetrahedron 1996, 52, 5833-5844; Kissman, H. M.; Farnsworth, D. W.; Witkop, B. J. Am. Chem. Soc. 1952, 74, 3948-3949; Smith, P. A. S.; Rowe, C. D.; Hansen, D. W., Jr. Tetrahedron Lett. 1983, 24, 5169-5172).

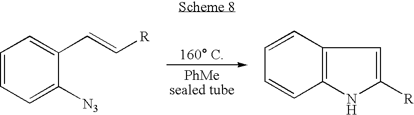

In another example of indole synthesis in Scheme 9, the Hemetsberger procedure for preparing indole-2-carboxylic acid involves thermolysis of α-azidocinnamate, which is from the condensation of aryl aldehyde and azidoacetate (Moody, C. J. J. Chem. Soc., Perkin 1: 1984, 1333-1337).

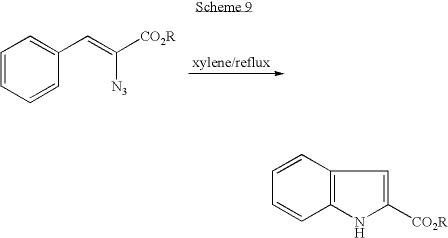

Thyagarajan has reported the synthesis of 2,3-disubstituted indoles from arylpropynylamine via N-oxidation using mCPBA and sequential sigmatropic rearrangement, Scheme 10 (Thyagarajan, B. S.; Hillard, J. B.; Reddy, K. V.; Majumdar, K. C. Tetrahedron Lett. 1974, 1999-2002).

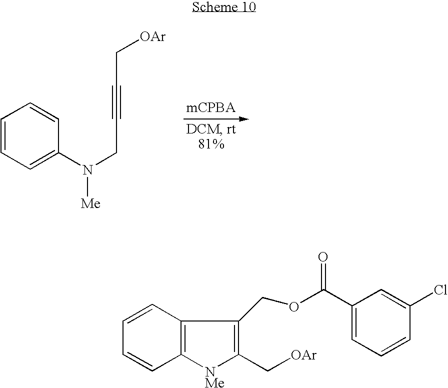

Allenylphenylamine was used to prepare 2-vinyl indoles in Scheme 11. The scope of the reaction, however, is limited to vinylindoles (Balasubramanian, T.; Balasubramanian, K. K. J. Chem. Soc., Chem. Commun. 1994, 1237-1238).

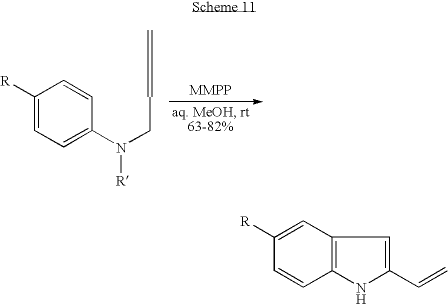

Gasssman has reported indole synthesis using a [2,3]-sigmatropic rearrangement from chlorosulfonium salt and aniline, Scheme 12 (Gassman, P. G.; Van Bergen, T. J.; Gilbert, D. P.; Cue, B. W., Jr. J. Am. Chem. Soc. 1974, 96, 5495-5508; Gassman, P. G.; Gruetzmacher, G.; Van Bergen, T. J. J. Am. Chem. Soc. 1974, 96, 5512-5517; Gassman, P. G.; Van Bergen, T. J. Org. Synth. 1977, 56, 72-77).

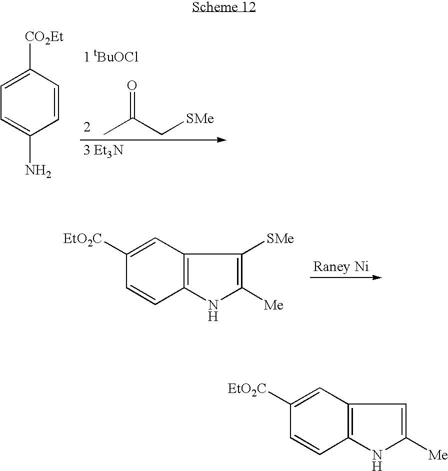

The Fürstner indole synthesis as shown in Scheme 13 involves Ti-induced cyclization of an oxo amide to give 2,3-disubstituted indoles (Fürstner, A.; Hupperts, A. J. Am. Chem. Soc. 1995, 117, 4468-4475; Fürstner, A.; Hupperts, A.; Seidel, G. Org. Synth. 1999, 76, 142-150; Fürstner, A.; Ptock, A.; Weintritt, H.; Goddard, R.; Krueger, C. Angew. Chem., Int. Ed. 1995, 34, 678-681).

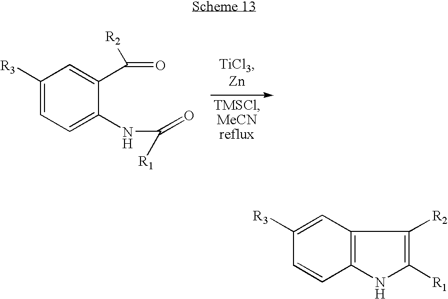

Castro and co-works developed the reaction of copper acetylenide with o-iodoaniline or copper mediated reaction of cyclization of o-alkynylanilines to synthesize 2-substituted indoles, as shown in Scheme 14 (Stephens, R. D.; Castro, C. E. J. Org. Chem. 1963, 28, 3313-3315; Castro, C. E.; Gaughan, E. J.; Owsley, D.C. J. Org. Chem. 1966, 31, 4071-4078; Castro, C. E.; Havlin, R.; Honwad, V. K.; Malte, A. M.; Moje, S. W. J. Am. Chem. Soc. 1969, 91, 6464-6470).

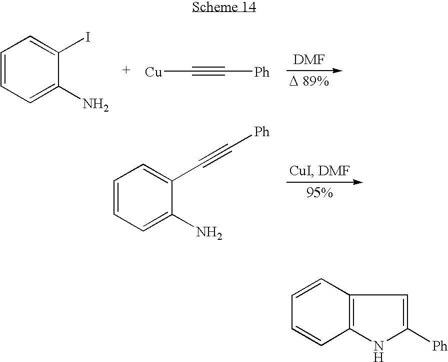

As shown in Scheme 15, Yamanaka and Sakamoto developed a Pd-catalyzed version of the reaction (Sakamoto, T.; Kondo, Y.; Yamanaka, H. Heterocycles 1988, 27, 2225-2249). When both copper and palladium were utilized in the catalytic system, an efficient one-pot reaction was developed (Sakamoto, T.; Kondo, Y.; Iwashita, S.; Nagano, T.; Yamanaka, H. Chem. Pharm. Bull. 1988, 36, 1305-1308). Other variations of this reaction involve coupling between o-aminophenylacetylene and vinyl triflates followed by cyclization (Cacchi, S.; Carnicelli, V.; Marinelli, F. J. Organomet. Chem. 1994, 475, 289-296).

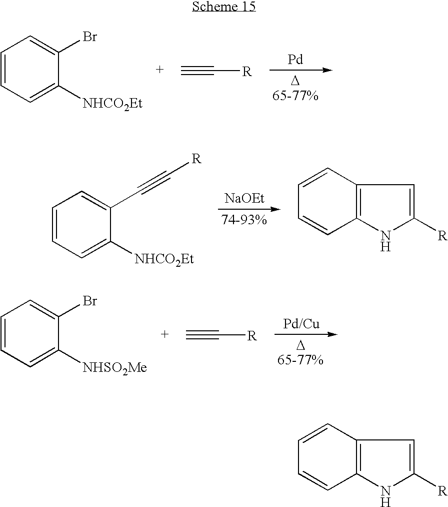

Larock reported Pd-catalyzed indole synthesis reaction between o-iodoaniline and internal alkynes (Scheme 16) (Larock, R. C.; Yum, B. K. J. Am. Chem. Soc. 1991, 113, 6689-6690).

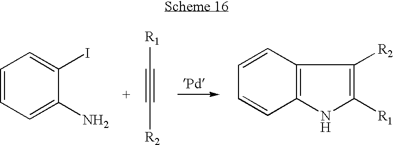

Iodine-mediated cyclization of N,N-dialkyl-2-(1-alkynyl)anilines to give N-alkyl-3-iodoindoles has also been reported (Scheme 17; Yue, D.; Larock, R. C. Org. Lett. 2004, 6, 1037-1040).

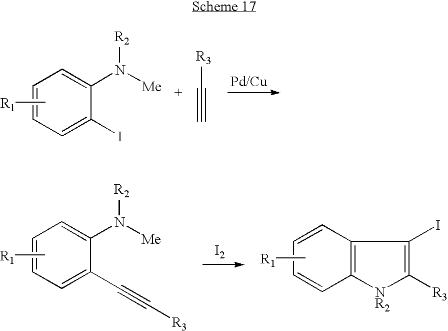

In yet another example, ring contraction-dimerization of 4H-3,1-benzothiazines was used to synthesize 2-substituted indoles using a two-step sequence, Scheme 18 (El-Desoky, S. I.; Kandeel, E. M.; Abd-el-Rahman, A. H.; Schmidt, R. R. J. Heterocycl. Chem. 1999, 36, 153-160).

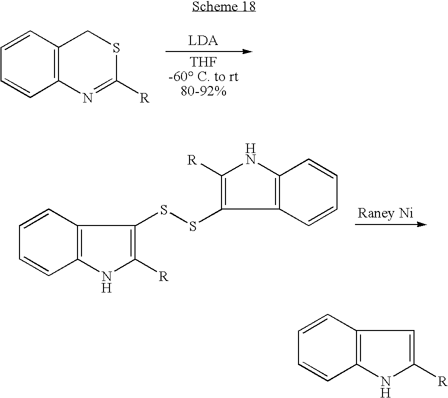

As shown in Scheme 19, Jamart-Grègoire and co-workers have reported 2-substituted indoles by cyclization of halogenated aryl imines through a benzyne intermediate (Kuehn-Caubere, C.; Rodriguez, I.; Pfeiffer, B.; Renard, P.; Caubere, P. J Chem. Soc., Perkin 1 1997, 2857-2862).

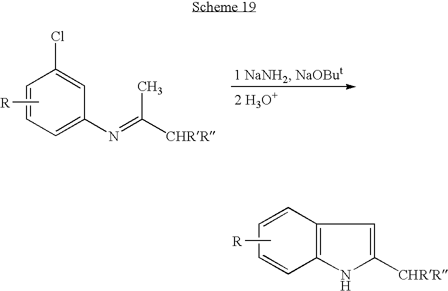

2-Substituted indoles have also been reported to be obtainable by modification of unsubstituted indoles, mainly using directed lithiation methodologies as shown in Scheme 20 (Sundberg, R. J.; Russell, H. F. J. Org. Chem. 1973, 38, 3324-3330; Saulnier, M. G.; Gribble, G. W. J. Org. Chem. 1982, 47, 2810-2812).

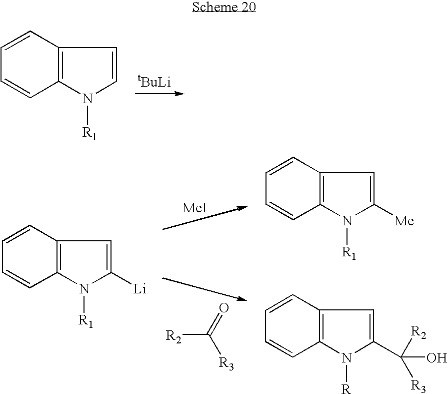

Direct C—H activation is also possible for introducing 2-aryl substitution by reacting an indole with aryl iodide under palladium-catalyzed conditions, shown in Scheme 21 (Sames, D.; Sezen, B.; Lane, B. S. WO 2004069394, 2004; Lane, B. S.; Sames, D. Org. Lett. 2004, 6, 2897-2900; Sezen, B.; Sames, D. J. Am. Chem. Soc. 2003, 125, 5274-5275).

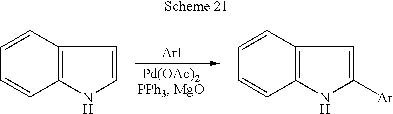

Recently, Bisseret and co-workers reported on the preparation of an N-acetyl-2-arylindole using N-acetylated ortho-gem-dibromovinylaniline and p-methoxyphenylboronic acid in the presence of a palladium catalyst and dppf (1,1′-bis(diphenylphosphino)ferrocene) as a ligand, Scheme 22. However, the yield of this example was moderate, 52% yield) (Thielges, S.; Meddah, E.; Bisseret, P.; Eustache, J. Tetrahedron Lett. 2004, 45, 907-910). Moreover, the amino group of the ortho-gem-dibromovinylaniline with activated with an acetyl group prior to successful tandem cyclization-coupling, followed by a deprotection step to remove the acetyl group from the final product. Protection and deprotection add undesirable steps to the synthesis since N—Ac indoles are usually not synthetic targets.

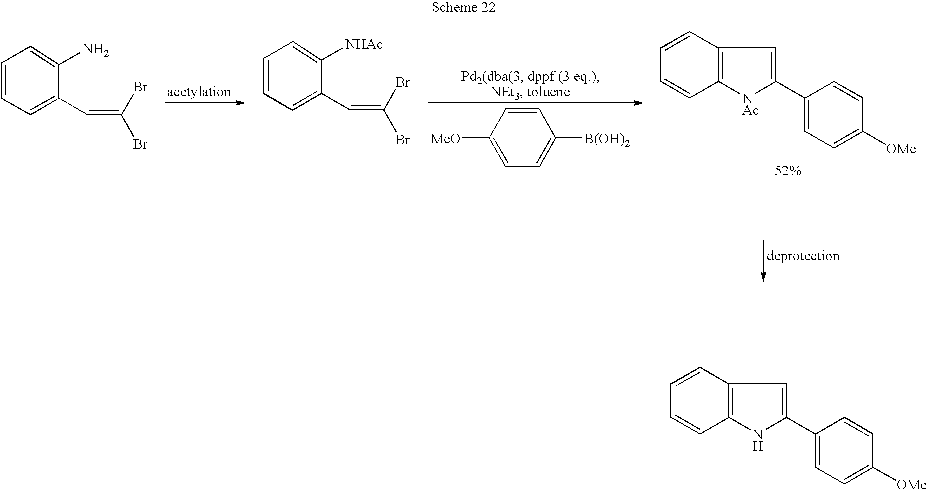

In yet another example as shown in Scheme 23, 2-haloanilines were condensed with a ketone to form enamines, which were in situ cyclized using Pd-catalyzed C—C bond formation (Chen, C.-y.; Lieberman, D. R.; Larsen, R. D.; Verhoeven, T. R.; Reider, P. J. J. Org. Chem. 1997, 62, 2676-2677; Nazare, M.; Schneider, C.; Lindenschmidt, A.; Will, D. W. Angew. Chem., Int. Ed. 2004, 43, 4526-4528).

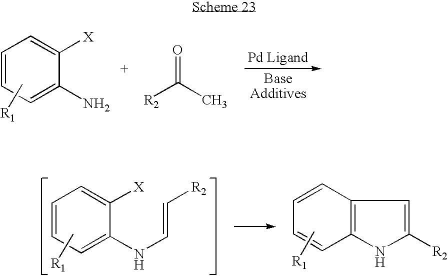

In view of the above, there remains a need for novel and versatile processes for synthesizing substituted indole compounds, in particular 2-substituted indole compounds, such as 1,2-substituted indoles, 2,3-substituted indoles, and 1,2,3-substituted indoles. The development and implementation of such processes could simplify the preparation of commercially important indole compounds.

One such commercially important indole compound is the lipid metabolism regulator fluvastatin (sold as Lescol®), the structure of which is shown below in its sodium salt form:

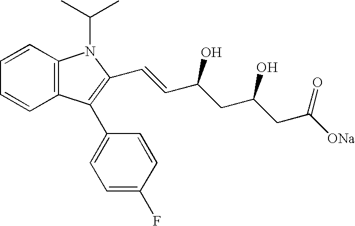

Fluvastatin is currently sold as a racemate of two erthryo enantiomers of which one exerts the pharmacological activity. Fluvastatin has two optical enantiomers, an active 3R,5S and an inactive 3S,5R form (Compendium of Pharmaceuticals and Specialities (CPS), 2005, 40^{th} Edition, Canadian Pharmacists Association). Synthetic methods exist for the synthesis of the racemic version of the drug (Repic, O.; Prasad, K.; and Lee, G. T. Organic Process Research & Development 2001, 5, 519-527), however, processes for making the enantiopure drug are highly desired.

Another such commercially important indole compound is the following potent and selective kinase insert domain receptor (KDR) inhibitor 3-[5-[[4-(methylsulfonyl)-1-piperazinyl]methyl]-1H-indole-2-yl]quinolin-2(1H)-one: 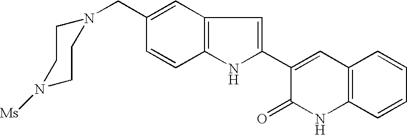
KDR belongs to the class of enzymes known as tyrosine kinases, which are believed to play a critical role in signal transduction in a number of cellular functions. Tyrosine kinases have been implicated in a wide range of diseases and conditions. KDR in particular is a tyrosine kinase that has a high affinity for vascular endothelial growth factor, and is believed to be a primary mediator of tumor induced angiogenesis. Therefore, compounds which inhibit, modulate, or regulate the I<DR receptor are useful for preventing and treating tumor induced angiogenesis. The KDR inhibitor 3-[5-[[4-(methylsulfonyl)-1-piperazinyl]methyl]-1H-indole-2-yl]quinolin-2(1H)-one shown above has recently been identified as a clinical candidate for use in cancer treatment (Kuethe, J. T. et. al. J. Org. Chem. 2005, 70, 2555-2567; Payack, J. F. et. al. J. Org. Chem. 2005, 70, 175-178; Wong, A. et al. J. Org. Chem. 2004, 69, 7761-7764; and references therein).

Methods for synthesizing 3-[5-[[4-(methylsulfonyl)-1-piperazinyl]methyl]-1H-indole-2-yl]quinolin-2(1H)-one are known in the art and provide the desired compound in 55-60% overall yield (Payack, J. F. et. al. J. Org. Chem. 2005, 70, 175-178; Wong, A. et. al. J. Org. Chem. 2004, 69, 7761-7764; Kuethe, J. T. et. al. J. Org. Chem. 2005, 70, 2555-2567; De-Feo-Jones, D. et. al. U.S. patent US2002/0041880 A1, 2002; Fraley, M. E. et. al. U.S. Pat. No. 6,306,874 B1, 2001; Merck&Co., Inc. WO 087651, 2004). Processes for synthesizing 3-[5-[[4-(methylsulfonyl)-1-piperazinyl]methyl]-1H-indole-2-yl]quinolin-2(1H)-one in higher yields are highly desired.

### SUMMARY OF THE INVENTION

Included in the scope of the invention is a process for the preparation of 2-substituted indole compounds. In particular, a process for the preparation of 2-substituted indole compounds is provided wherein the 2-substituent designated as R₄ is bonded to the 2-position of the indole ring via a C—C bond, the process comprising reacting an ortho-gem-dihalovinylaniline compound of the formula: 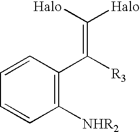
wherein Halo comprises Br, Cl, or I, R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents, and R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; with an organoboron reagent selected from the group consisting of a boronic ester of R₄, a boronic acid of R₄, a boronic acid anhydride of R₄, a trialkylborane of R₄ and a 9-BBN derivative of R₄; in the presence of a base, a palladium metal pre-catalyst and a ligand under reaction conditions effective to form the 2-substituted indole compound.

Also included within the scope of the invention is a process for the preparation of a compound comprising within its structure a 2-substituted indole moiety of formula (I), 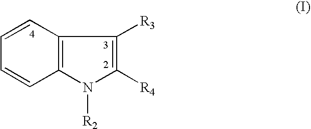
wherein R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond; and R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents, and R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; the process comprising reacting an ortho-gem-dihalovinylaniline compound of formula (II) 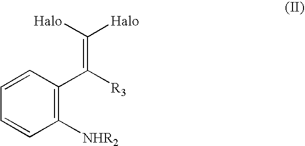
wherein Halo comprises Br, Cl, or I; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents, and R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; with an organoboron reagent selected from the group consisting of a boronic ester of R₄, a boronic acid of R₄, a boronic acid anhydride of R₄, a trialkylborane of R₄ and a 9-BBN derivative of R₄; in the presence of a base, a palladium metal pre-catalyst and a ligand under reaction conditions effective to form the 2-substituted indole compound.

In yet another aspect of the present invention is provided a process for the preparation of a 2-substituted indole compound of formula (IV) 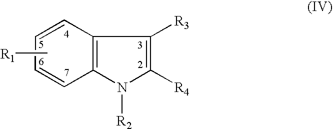
wherein each of the one or more R₁ substituents is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the indole ring; all of which are optionally substituted with one or more suitable substituents at one or more substitutable positions; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents; R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond; the process comprising reacting an ortho-gem-dihalovinylaniline compound of formula (V) 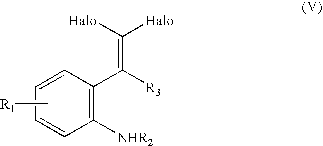
wherein R₁, R₂ and R₃ are as defined above, and Halo comprises bromo, chloro, or iodo; with an organoboron reagent selected from the group consisting of a boronic ester of R₄, a boronic acid of R₄, a boronic acid anhydride of R₄, a trialkylborane of R₄ and a 9-BBN derivative of R₄; in the presence of a base, a palladium metal pre-catalyst and a ligand under reaction conditions effective to form the 2-substituted indole compound.

Also included within the scope of the invention is a process for the palladium-catalyzed tandem intramolecular C—N bond formation and intermolecular C—C bond formation between an ortho-gem-dihalovinylaniline compound of formula (V) 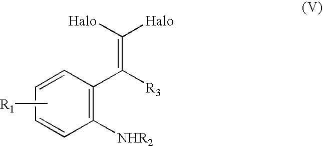
wherein each R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the indole ring; all of which are optionally substituted with one or more suitable substituents at one or more substitutable positions; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents; R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and Halo comprises bromo, chloro, or iodo; preferably chloro or bromo; with an organoboron reagent selected from the group consisting of a boronic ester of R₄, a boronic acid of R₄, a boronic acid anhydride of R₄, a trialkylborane of R₄ and a 9-BBN derivative of R₄, wherein R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond, for the preparation of a 2-substituted indole of formula (IV) 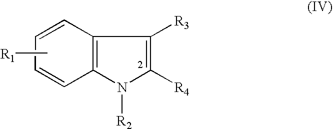
wherein R₁, R₂, R₃ and R₄ are as defined above, the process comprising reacting the ortho-gem-dihalovinylaniline compound of formula (V) with the organoboron reagent in the presence of a base, a palladium metal pre-catalyst and a ligand under reaction conditions effective to afford the tandem C—N and C—C bond formation between the ortho-gem-dihalovinylaniline compound of formula (V) and the organoboron reagent to afford the 2-substituted indole of formula (IV).

In yet another aspect of the present invention is a process for the preparation of an ortho-gem-dibromovinylaniline compound of formula (V) 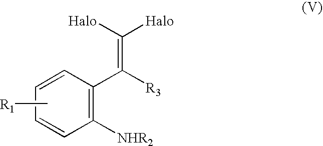
wherein each R₁, is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₂ is H, and R₃ is H, CF₃ or alkynyl optionally substituted at one or more positions with one or more suitable substituents, and Halo comprises bromo, said process comprising the steps of: (a) reacting a nitrobenzaldehyde compound (R₃═H) of formula (VI) or a trifluoroacetylnitrobenzene (R₃═CF₃) or alkynylcarbonynitrobenzene (R₃-alkynyl). 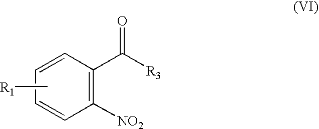
wherein R₁ is as defined above, with CBr₄ and PPh₃ under conditions effective to generate in situ the ortho-gem-dibromovinyl compound of formula (VII) 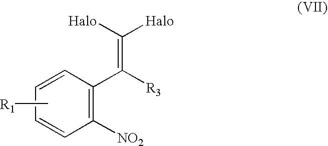
wherein R₁ and R₃ are as defined above and Halo is bromo; and (b) reducing the compound of formula (VII) under conditions effective to reduce the nitro group of the compound of formula (VII) without affecting the functional groups present in the compound, to afford the compound of formula (V).

In yet another aspect of the present invention is a process for the preparation of an ortho-gem-dihalovinylaniline compound of formula (V) 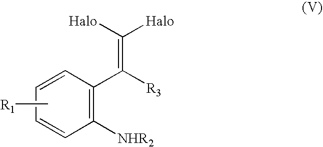
wherein each R₁, is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₂ is H and R₃ is H, alkyl, or alkynyl optionally substituted at one or more positions with one or more suitable substituents, and Halo comprises chloro, said process comprising the steps of: (a) reacting a nitrobenzaldehyde or ketone compound of formula (VI) 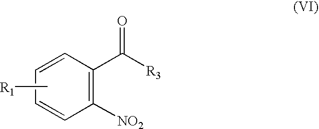
wherein R₁ and R₃ are as defined above, with about 2 or more equivalents of CHCl₃ and PPh₃ in the presence of about 2 or more equivalents of KO^{t}Bu (all equivalents relative to the starting material of formula (VI)) under conditions effective to generate in situ the ortho-gem-dichlorovinyl compound of formula (VII) 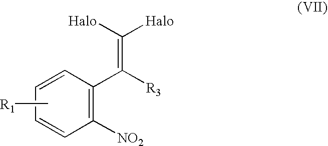
wherein R₁ and R₃ are as defined above and Halo is chloro; and (b) reducing the compound of formula (VII) under conditions effective to reduce the nitro group of the compound of formula (VII), without affecting the functional groups present in the compound, to afford the compound of formula (V). In a preferred embodiment, the reducing agent is SnCl₂.2H₂O and H₂ catalyzed by platinum on carbon doped with vanadium.

Also included within the scope of the invention is the use of a compound of formula (V) 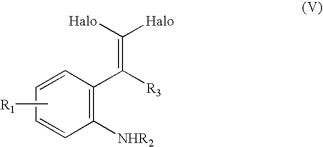
wherein Halo comprises Br, Cl, or I; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents; R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; in the preparation of a compound containing a 2-substituted indole compound of formula 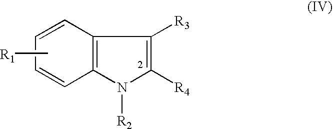
wherein R₁, R₂, and R₃ are as defined above, R₄ comprises monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond.

Also included within the scope of the invention are the following novel 2-substituted indoles, and their salts:

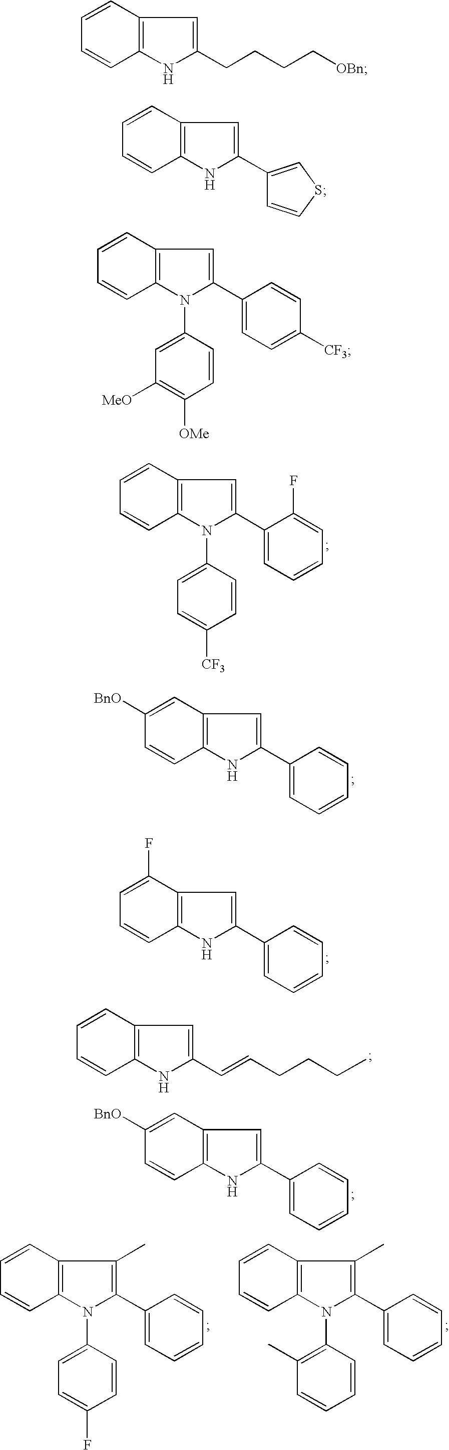

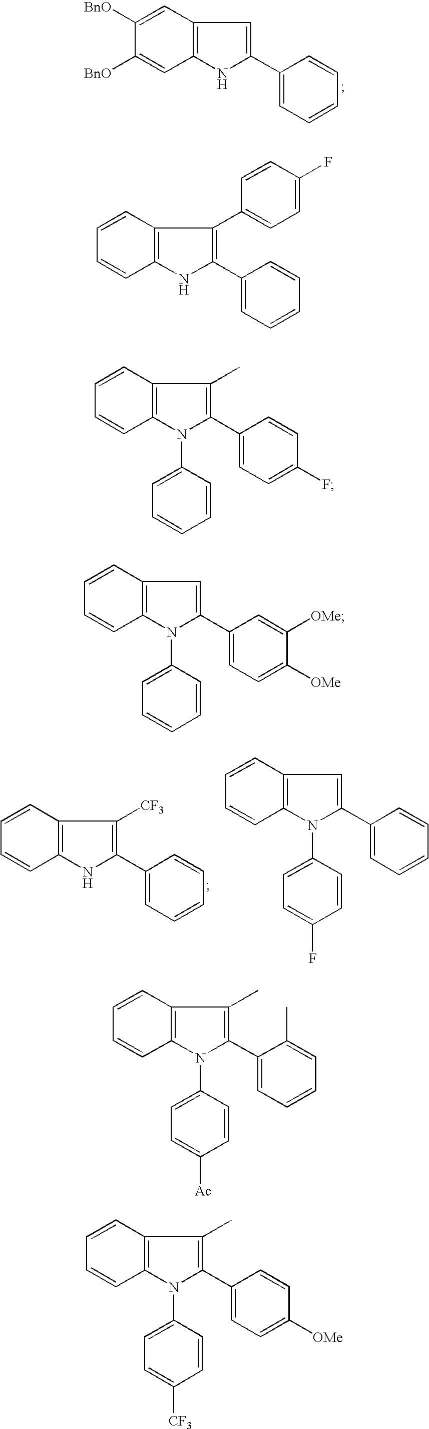

Also contained within the invention are the novel 2-substituted indoles and their salts when prepared by a process of the present invention.

Also contained within the present invention are the following novel ortho-gem-dihalovinylaniline compounds and their salts:

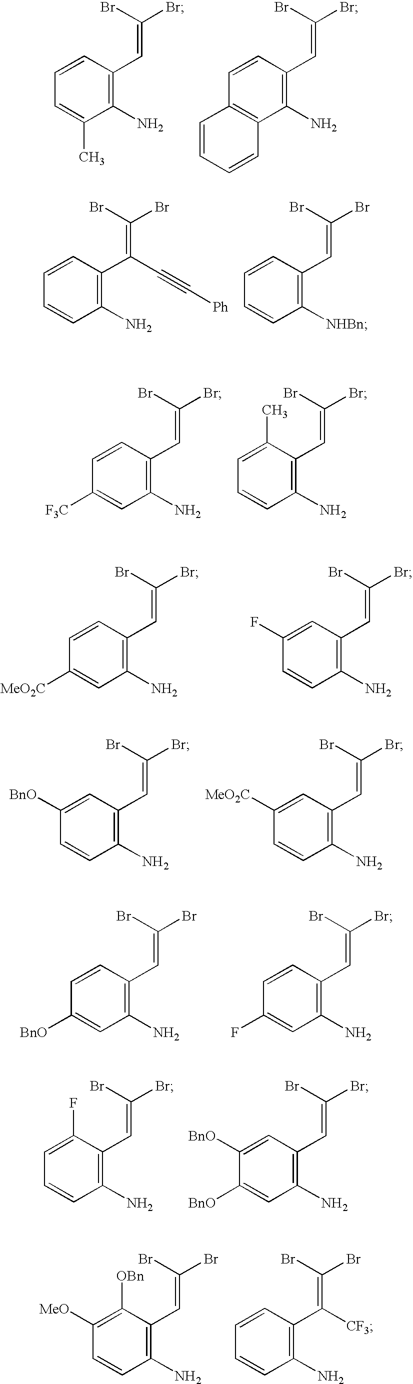

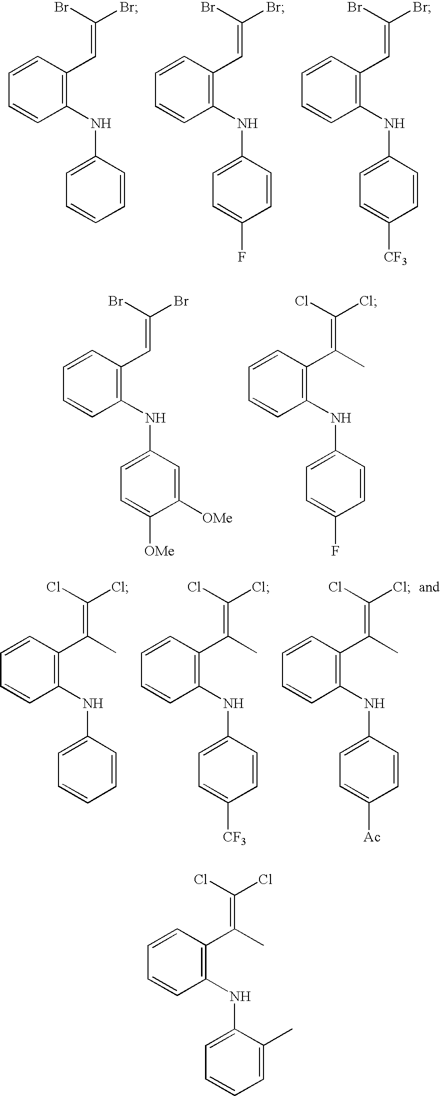

Novel ortho-gem-dihalovinylaniline compounds when prepared by a process of the present invention are likewise encompassed within the present invention. Novel ortho-gem-dihalovinylaniline compounds are useful in the preparation of 2-substituted indoles as described herein.

In yet another aspect of the present invention is a process for the preparation of an ortho-gem-dihalovinylaniline compound of formula (V) 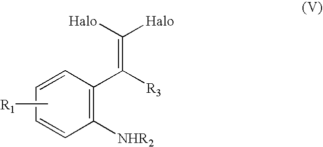
wherein each of the one or more R₁ substituents is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₂ comprises H; R₃ comprises alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and Halo comprises bromo or chloro, said process comprising the steps of: (a) converting a ketone of formula (VIII) 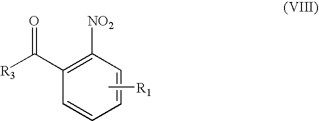
wherein R₁ and R₃ are as defined above into its corresponding olefin derivative of formula (IX) under conditions effective to generate the corresponding olefin derivative of formula (IX) 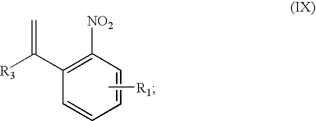
(b) halogenating the olefin derivative of formula (IX) under conditions effective to generate the dihalogen compound of formula (X) 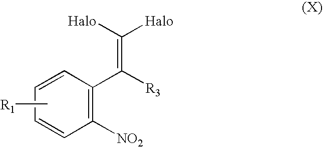
wherein R₁, Halo, and R₃ are defined above; and
(c) reducing the compound of formula (X) under conditions effective to reduce the nitro group of the compound of formula (X) without affecting the functional groups present in the compound, to afford the compound of formula (V).

In yet another aspect of the present invention is provided a method for the preparation of N-arylaniline compounds of formula (XI) 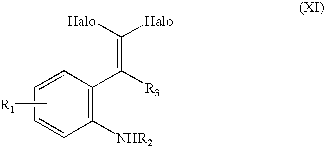
wherein Halo comprises Br, Cl, or I; R₂ comprises aryl which is optionally substituted at one or more substitutable positions with one or more suitable substituents; R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (XI); all of which are optionally substituted with one or more suitable substituents at one or more substitutable positions; said process comprising the steps of:
(a) reacting a compound of formula (V) 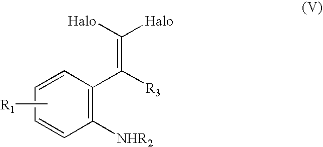
wherein Halo, R₁, R₃ are as defined in Formula (XI) above and R₂ is H, with an organoboron reagent comprising a boronic acid, boronic acid anhydride or BF₃⁻ salt of R₂ in the presence of at least about 1, more preferably at least about 1.5 equivalents of a copper (II) catalyst (relative to the compound of formula (V)), at least about 0.3 equivalents of a C₈-C₂₀ fatty acid, preferably myristic acid (relative to the compound of formula (V)), molecular oxygen, and a non-nucleophilic base, such as lutidine or collidine, at a reaction temperature of between about 40° C. and 60° C., under conditions effective to form a C—N bond between formula (V) and the R₂ group of the organoboron reagent, to afford the N-arylaniline compounds of formula (XI).

In yet another aspect of the present invention is provided a method for the preparation of N-alkylaniline compounds of formula (XI) 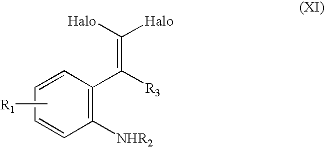
wherein Halo comprises Br, Cl, or I; R₂ comprises alkyl which is optionally substituted at one or more substitutable positions with one or more suitable substituents; R₃ comprises H, alkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (XI); all of which are optionally substituted with one or more suitable substituents at one or more substitutable positions; said process comprising the steps of:
reacting a compound of formula (V) 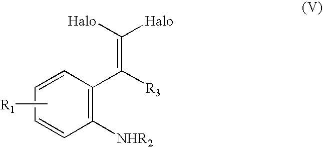
wherein Halo, R₁, R₃ are as defined in Formula (XI) above and R₂ is H, with a suitable alkylating agent, such as alkyl iodide or alkylbromide, under conditions effective to form a C—N bond between formula (V) and the alkyl group of the alkyl halide, to afford the N-alkylaniline compounds of formula (XI). These compounds are useful for the synthesis of 2-substituted indoles of the present invention as described herein.

In yet another aspect, the invention provides the following novel compounds 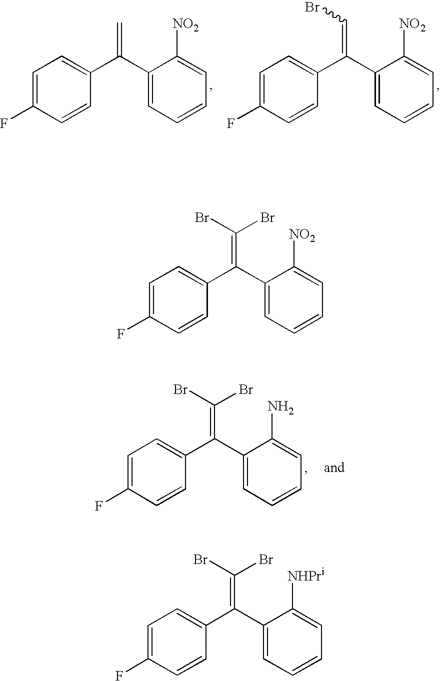
the use thereof for the synthesis of fluvastatin or a pharmaceutically acceptable salt thereof shown below in its sodium salt form:

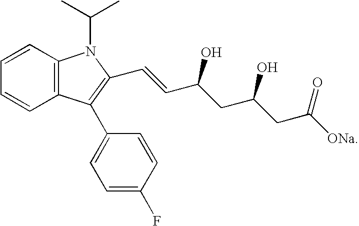

In yet another aspect, the invention provides the following novel compounds 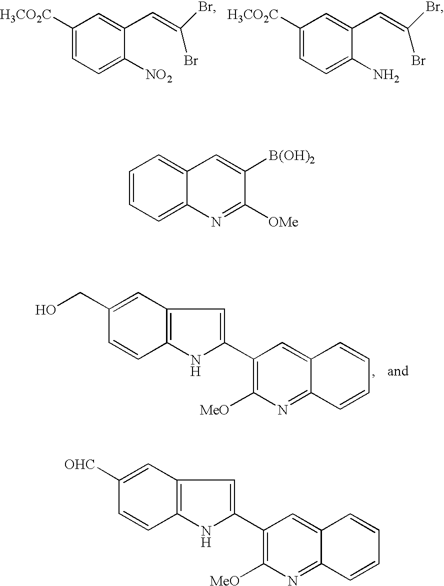
and the use thereof for the synthesis of the KDR inhibitor 3-[5-[[4-(methylsulfonyl)-1-piperazinyl]methyl]-1H-indole-2-yl]quinolin-2(1H)-one:

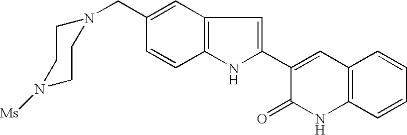

These and other aspects will become apparent upon reading the following detailed description of the invention.

### DETAILED DESCRIPTION OF EMBODIMENTS

The present invention provides novel, versatile and efficient processes and conditions for the palladium-catalyzed chemical synthesis of a variety of 2-substituted indole compounds, including 2,4-disubstituted, 1,2-disubstituted, and 1,2,3-trisubstituted indoles, from inexpensive starting materials that can be easily prepared in large quantities. Moreover, the palladium pre-catalyst loadings useful in the present invention are low, in some embodiments about 1% or less, and the processes typically afford yields of 2-substituted indoles in about the 70-90% range. The novel process can allow for the rapid access and the ease of production of diversified indoles, their analogs and their derivatives.

The processes of the present invention further provide reaction conditions, and starting materials which are precursors for the preparation of 2-substituted indoles, as well as novel processes and conditions for the preparation of the precursor materials.

The present invention further provides a highly modular method for palladium-catalyzed tandem carbon-nitrogen/carbon-carbon bond formation between an ortho-gemdihalogen substituted vinylaniline compound with an organoboron reagent in the presence of a palladium pre-catalyst and a ligand to afford 2-substituted indole compounds.

The present invention also provides novel 2-substituted indole compounds prepared by the novel processes of the present invention as well as novel ortho-gem-dihalovinylaniline derivatives for the production of 2-substituted indoles.

The present invention further provides novel methods for the copper-mediated C—N coupling of anilines and arylboronic acids to prepare N-aryl-ortho-gem-dihalovinylaniline compounds that are useful as intermediates in the processes of the present invention for the preparation of 2-substituted indoles.

The present invention further provides novel methods for the preparation of ortho-gem-dihalovinylaniline compounds as intermediates in the processes of the present invention for the preparation of 2-substituted indoles.

The present invention further provides a novel method for the synthesis of the 2-substituted indole, Fluvastatin and its salts.

The present invention further provides a novel method for the synthesis of the KDR inhibitor 3-[5-[[4-(methylsulfonyl)-1-piperazinyl]methyl]-1H-indole-2-yl]quinolin-2(1H)-one.

Therefore, in one embodiment of the present invention is provided a process for the preparation of 2-substituted indole compounds wherein the 2-substituent (generally designated as an R₄ group) is bonded to the 2-position of the indole ring via a C—C bond, which comprises reacting an ortho-gem-dihalovinylaniline compound of the formula: 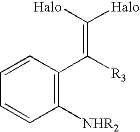
wherein Halo comprises Br, Cl, or I, R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents, and R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; with an organoboron reagent selected from the group consisting of a boronic ester of R₄, a boronic acid of R₄, a boronic acid anhydride of R₄, a trialkylborane of R₄ and a 9-BBN derivative of R₄; in the presence of a base, a palladium metal pre-catalyst and a ligand under reaction conditions effective to form a 2-substituted indole compound, wherein R₄ is directly bonded to the 2-position of the indole ring via a C—C bond.

In one embodiment, R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents.

In another embodiment of the present invention is provided a process for the preparation of a compound comprising within its structure a 2-substituted indole moiety of formula (I), 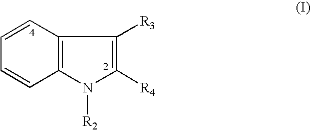
wherein R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents, and R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; the process comprising reacting an ortho-gem-dihalovinylaniline compound of formula (II) 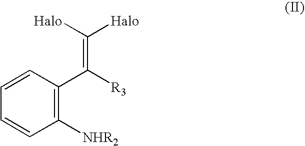
wherein Halo comprises Br, Cl, or I; R₂ is H, alkyl, cycloalkyl, aryl, heteroaryl, aryl-loweralkyl-, or heteroaryl-loweralkyl-, all of which are optionally substituted at one or more substitutable positions with one or more suitable substituents (preferably H, Benzyl (Bn), or alkyl, wherein said alkyl and benzyl group are optionally substituted at one or more substitutable positions with one or more suitable substituents); and R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; with an organoboron reagent selected from the group consisting of a boronic ester of R₄, a boronic acid of R₄, a boronic acid anhydride of R₄, a trialkylborane of R₄ and a 9-BBN derivative of R₄; in the presence of a base, a palladium metal pre-catalyst and a ligand under reaction conditions effective to form the 2-substituted indole compound.

In yet another embodiment of the present invention is provided a process for the preparation of a 2-substituted indole compound of formula (IV) 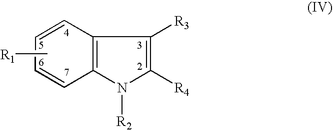
wherein each R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the indole ring; all of which are optionally substituted with one or more suitable substituents at one or more substitutable positions; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents; R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond; the process comprising reacting an ortho-gem-dihalovinylaniline compound of formula (V) 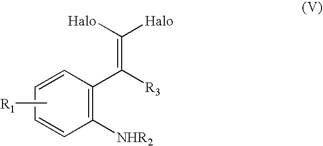
wherein R₁, R₂ and R₃ are as defined above, and Halo comprises bromo, chloro, or iodo; with an organoboron reagent selected from the group consisting of a boronic ester of R₄, a boronic acid of R₄, a boronic acid anhydride of R₄, a trialkylborane of R₄ and a 9-BBN derivative of R₄; in the presence of a base, a palladium metal pre-catalyst and a ligand under reaction conditions effective to form the 2-substituted indole compound.

In yet another embodiment of the present invention is a process for the palladium-catalyzed tandem intramolecular C—N bond formation and intermolecular C—C bond formation between an ortho-gem-dihalovinylaniline compound of formula (V) 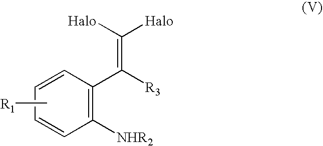
wherein each R₁ is independently selected from the group consisting of H, fluoro, lower alkyl, lower alkenyl, lower alkoxy, aryloxy, lower halo alkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the indole ring; all of which are optionally substituted with one or more suitable substituents at one or more substitutable positions; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents; R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and Halo comprises Iodo, chloro, or bromo; with an organoboron reagent selected from the group consisting of a boronic ester of R₄, a boronic acid of R₄, a boronic acid anhydride of R₄, a trialkylborane of R₄ and a 9-BBN derivative of R₄, wherein R₄ is selected from the group consisting of monocyclic aromatic, polycyclic aromatic, monocyclic heteroaromatic, polycyclic heteroaromatic, alkyl, cycloalkyl, and alkenyl, all of which are optionally substituted at one or more substitutable positions with one or more suitable substituents, and wherein R₄ is bonded to the 2-position of the indole ring via a C—C bond, for the preparation of a 2-substituted indole of formula (IV) 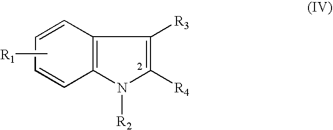
wherein R₁, R₂, R₃ and R₄ are as defined above, the process comprising reacting the ortho-gem-dihalovinylaniline compound of formula (V) with the organoboron reagent in the presence of a base, a palladium metal pre-catalyst and a ligand under reaction conditions effective to afford the tandem C—N and C—C bond formation between the ortho-gem-dihalovinylaniline compound of formula (V) and the organoboron reagent to afford the 2-substituted indole of formula (IV).

As used in the context of the present invention, the various chemical terms are to be given their ordinary meaning as would be understood by persons skilled in the art, unless provided otherwise.

The following chemical terms presently described apply to all compounds and processes disclosed herein, unless provided otherwise.

The term “suitable substituent” as used in the context of the present invention is meant to include independently H; hydroxyl; cyano; alkyl, such as lower alkyl, such as methyl, ethyl, propyl, n-butyl, t-butyl, hexyl and the like; alkoxy, such as lower alkoxy such as methoxy, ethoxy, and the like; aryloxy, such as phenoxy and the like; vinyl; alkenyl, such as hexenyl and the like; alkynyl; formyl; haloalkyl, such as lower haloalkyl which includes CF₃, CCl₃ and the like; halide; aryl, such as phenyl and napthyl; heteroaryl, such as thienyl and furanyl and the like; amide such as C(O)N(CH₃)₂ and the like; acyl, such as C(O)—C₆H₅, and the like; ester such as —C(O)OCH₃ the like; ethers and thioethers, such as O-Bn and the like; amino; thioalkoxy; phosphino and the like. It is to be understood that a suitable substituent as used in the context of the present invention is meant to denote a substituent that does not interfere with the formation of the desired product by the claimed processes of the present invention.

As used in the context of the present invention, the term “loweralkyl” as used herein either alone or in combination with another substituent means acyclic, straight or branched chain alkyl substituent containing from one to six carbons and includes for example, methyl, ethyl, 1-methylethyl, 1-methylpropyl, 2-methylpropyl, and the like. A similar use of the term is to be understood for “lower alkoxy”, “lower thioalkyl”, “lower alkenyl” and the like in respect of the number of carbon atoms. For example, “lower alkoxy” as used herein includes methoxy, ethoxy, t-butoxy.

The term “aryl” as used herein, either alone or in combination with another substituent, means an aromatic monocyclic system containing 6 carbon atoms or an aromatic bicyclic system containing 10 carbon atoms. For example, the term “aryl” includes a phenyl or a napthyl ring.

The term “heteroaryl” as used herein, either alone or in combination with another substituent means a 5, 6, or 7-membered unsaturated heterocycle containing from one to 4 heteroatoms selected from nitrogen, oxygen, and sulphur and which form an aromatic system

The term “cycloalkyl” as used herein, either alone or in combination with another substituent, means a cycloalkyl substituent that includes for example, but is not limited to, cyclopropyl, cyclobutyl, cyclopentyl, cyclohexyl and cycloheptyl.

The term “cycloalkyl-alkyl-” as used herein means an alkyl radical to which a cycloalkyl radical is directly linked; and includes, but is not limited to, cyclopropylmethyl, cyclobutylmethyl, cyclopentylmethyl, 1-cyclopentylethyl, 2-cyclopentylethyl, cyclohexylmethyl, 1-cyclohexylethyl and 2-cyclohexylethyl. A similar use of the “alkyl” term is to be understood for aryl-alkyl-, heteroaryl-alkyl-, and the like as used herein. For example, the term “aryl-alkyl-” means an alkyl radical, to which an aryl is bonded. Examples of aryl-alkyl- include, but are not limited to, benzyl (phenylmethyl), 1-phenylethyl, 2-phenylethyl and phenylpropyl.

As used herein, the term “heterocycle”, either alone or in combination with another radical, means a monovalent radical derived by removal of a hydrogen from a three- to seven-membered saturated or unsaturated (including aromatic) heterocycle containing from one to four heteroatoms selected from nitrogen, oxygen and sulfur. Examples of such heterocycles include, but are not limited to, azetidine, pyrrolidine, tetrahydrofuran, thiazolidine, pyrrole, thiophene, hydantoin, diazepine, imidazole, isoxazole, thiazole, tetrazole, piperidine, piperazine, homopiperidine, homopiperazine, 1,4-dioxane, 4-morpholine, 4-thiomorpholine, pyridine, pyridine-N-oxide or pyrimidine, and the like.

The term “alkenyl”, as used herein, either alone or in combination with another radical, is intended to mean an unsaturated, acyclic straight chain radical containing two or more carbon atoms, at least two of which are bonded to each other by a double bond. Examples of such radicals include, but are not limited to, ethenyl (vinyl), 1-propenyl, 2-propenyl, and 1-butenyl.

The term “alkynyl”, as used herein is intended to mean an unsaturated, acyclic straight chain radical containing two or more carbon atoms, at least two of which are bonded to each other by a triple bond. Examples of such radicals include, but are not limited to, ethynyl, 1-propynyl, 2-propynyl, and 1-butynyl.

The term “alkoxy” as used herein, either alone or in combination with another radical, means the radical —O—(C₁₋ₙ)alkyl wherein alkyl is as defined above containing 1 or more carbon atoms, and includes for example methoxy, ethoxy, propoxy, 1-methylethoxy, butoxy and 1,1-dimethylethoxy.

As used herein the term “heteroatom” means O, S or N.

Depending on the substitution on the starting material ortho-gem-dihalovinyl aniline compound and the organoboron reagent used in the processes of the present invention, the 2-substituted indole compound may bear additional substituents at various position of the indole ring, and it is to be understood that, in the context of the present invention, the term 2-substituted indoles is meant to include indoles that may include additional substituents at other positions in the structure. For example, in one embodiment, the present invention provides 2-substituted indoles that also have a substituent at the 4-position of the indole ring. In another embodiment, the present invention provides 2-substituted indoles that also bear a substituent at the 3-position of the indole ring and/or the 1-position of the indole ring. In one embodiment, the 2-substituted indoles additionally contain one or more substituents designated R₁, at the 4, 5, 6, and/or 7 position of the indole ring depending on the substitution pattern of the starting material ortho-gem-dihalovinylaniline to afford an indole of the following structure: 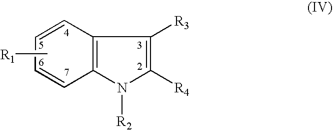
wherein each R₁ is independently selected from H; fluoro; alkyl, such as methyl, ethyl, propyl, n-butyl, t-butyl, and the like; alkenyl, and alkynyl; lower alkoxy, aryloxy, halo alkyl, —C(O)O-lower alkyl, monocyclic or polycyclic aryl or heteroaryl moiety, or R₁ is an alkenyl group bonded so to as to form a 4- to 20-membered fused monocycle or polycyclic ring with the indole ring; all of which are optionally substituted with one or more suitable substituents at one or more substitutable positions; R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents; R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond.

Additional specific examples of 2-substituted indoles that may be prepared by the processes of the present invention are shown in Tables 1 through 4, discussed in detail below.

In one embodiment of the novel processes, halo of the ortho-gem-dihalovinylaniline starting material of formula (II) or formula (V) comprises bromo or chloro. In another embodiment, halo of the ortho-gem-dihalovinylaniline compound of formula (II) or formula (V) comprises bromo. In other preferred embodiments, R₂ comprises H; or benzyl which is optionally substituted at one or more substitutable positions with one or more suitable substituents; or aryl which is optionally substituted at one or more substitutable positions with one or more suitable substituents, for example optionally substituted phenyl; or R₂ comprises alkyl such as methyl or ethyl, or the like. Use of an ortho-gem-dihalovinylaniline starting materials of formula (II) or formula (V) having an R₂ group such as H, or benzyl or alkyl or phenyl which are optionally substituted at one or more substitutable positions, advantageously does not significantly increase the acidity of the NH group to which they are bonded, unlike other groups such as N-acetyl groups, affording improved reactivity and acceptable yields in the process of the present invention. Since N-Acyl indoles are not usually final targets, and the use of N-Bn, N-alkyl or N-aryl indoles is more commonly observed, the claimed processes can be more straightforward and efficient.

In another preferred embodiment, R₂ comprises H and Halo of the ortho-gem-dihalovinylaniline starting material of formula (II) or formula (V) comprises bromo.

Methods for preparing ortho-gem-dihalovinylaniline compounds are known to those skilled in the art. For example, see Thielges, S.; Meddah, E.; Bisseret, P.; Eustache, J. Tetrahedron Lett. 2004, 45, 907-910 and Topolski, M. J. Org. Chem. 1995, 60, 5588-5594. Alternatively, the ortho-gem-dihalovinylaniline compounds of formula (II) or formula (V) may be prepared by the novel process of the present invention as are described and claimed below.

In one embodiment, the ortho-gem-dihalovinylaniline employed in the processes for the preparation of 2-substituted indoles comprises ortho-gem-dibromovinylaniline as described below in Example 1a, and the organoboron reagent of formula (III) comprises an reagent as follows:

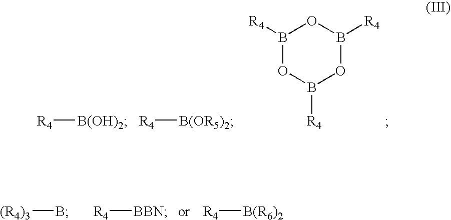

In one embodiment, the organoboron reagent comprises an organoboronic acid, such as phenylboronic acid, C₆H₅—B(OH)₂, which is optionally further substituted at one or more substitutable positions with one or more substituents such as methyl, OMe, CF₃, and the like. In another embodiment, the organoboron reagent comprises an organoboronic ester, such as a cyclic catechol ester, pinacol ester or ethylene glycol and the like. In one embodiment, R₅ of the organoboron ester may be a simple alkyl, such as methyl, ethyl, propyl and the like. Likewise, the organoboron reagent can comprise a 9-BBN derivative, such as n-HexBBN, or a trialkylboron reagent, such as Et₃B. In another embodiment, R₆ of the organoborane reagent maybe a cyclic or non-cyclic secondary alkyl group.

Many organoboron reagents are commercially available and methods for preparing organoboron reagents for use in the present invention are known to those skilled in the art. A description of general synthetic techniques used for preparing such organobornon reagents found in Miyaura, N.; Suzuki, A., Chem. Rev. 1995, 95, 2457-2483, and Suzuki, A. J. Organomet. Chem. 1999, 576, 147-168 is hereby incorporated herein by reference.

In one embodiment, the palladium pre-catalyst used in the processes for preparing 2-substituted indoles of the present invention is Pd(OAc)₂, Pd(PPh₃)₄, Pd₂(dba)₃, Pd(CH₃CN)₂Cl₂, PdCl₂, K₂PdCl₄, or Pd₂(dba)₃.HCCl₃. Palladium pre-catalysts are commercially available, and methods for preparing such palladium pre-catalysts are known to those skilled in the art. A description of general synthetic techniques used for preparing such pre-catalysts found in Jiro Tsuji, Palladium Reagents and Catalysts, John Wiley & Sons Ltd., 2004, is hereby incorporated herein by reference. In one embodiment, the pre-catalyst comprises Pd(OAc)₂ and the organoboron reagent comprises a boronic acid of R₄. In another embodiment, the pre-catalyst comprises Pd₂(dba)₃, and the organoboron reagent comprises a 9-BBN derivative of R₄.

The quantity of pre-catalyst which can be used can be any quantity which allows for the formation of the 2-substituted indole product. In one embodiment, the pre-catalyst is present in an amount of about 1 mole percent to about 5 mole percent relative to the ortho-gem-dihalovinylaniline compound used in the reaction. In another embodiment, the pre-catalyst is present in an amount of about 1 mole percent relative to the ortho-gem-dihalovinylaniline compound used in the reaction.

Ligands for use in the present processes for the preparation of 2-substituted indoles comprise a phosphorous-containing ligand or a nitrogen-containing carbenoid ligand, such as s-Phos, P(o-tol)₃, PPh₃, P(O—CF₃-Ph)₃, BINAP, tol-BINAP, dppm, dppe, dppp, dppb, dppf, Xanphos, BIPHEP, AsPh₃, and 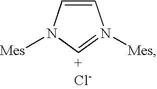
and the like. In one embodiment, the preferred ligand is s-Phos. Methods for preparing such ligands are well known to those skilled in the art. A description of general synthetic techniques used for preparing such ligands as found in Jiro Tsuji, Palladium Reagents and Catalysts, John Wiley & Sons Ltd., 2004, is hereby incorporated herein by reference.

The quantity of ligand which can be used can be any quantity which allows for the formation of the 2-substituted indole. In one embodiment, the ligand is present in amount of about 2 mole % to about 10 mole % relative to the ortho-gem-dihalovinylaniline compound used in the reaction. In another embodiment, the ligand is s-Phos and it is present in amount of about 1 mole % to 5 mole % relative to the ortho-gem-dihalovinylaniline compound. The preparation of s-Phos is described and referenced in the publication of Walker et al. Angew. Chem. Int. Ed. 2004, 43, 1871-1876 and Barder et al. J. Am. Chem. Soc. 2005, 127, 4685. the details of which are herein incorporated by reference. In one embodiment, s-Phos is employed as a ligand at about 2 mole % relative to the ortho-gem-dihalovinylaniline compound. In another embodiment, the ligand is s-Phos, used in combination with Pd(OAc)₂ as a pre-catalyst, and which are present in quantities of 2.5 mole % and 1 mole %, respectively. The ratio of s-Phos and Pd ranges from 1.5˜2.5:1.

In another embodiment of the processes of the present invention for the preparation of 2-substituted indoles, the base comprises an organic base or an inorganic base, such as a metal carbonate, a metal hydroxide, a metal phosphonate or a trialkylamine, and the like. In one embodiment, the base comprises K₂CO₃, Na₂CO₃, Cs₂CO₃, NaOH, K₃PO₄.H₂O, or NEt₃. In another embodiment, the base comprises K₃PO₄.H₂O. Additional bases for use with the present processes are known to those skilled in the art, for example, such as those disclosed in the publication of Miyaura et al. Chem. Rev. 1995, 95, 2457-2483, the details of which as relating to the bases is hereby incorporated herein by reference. In another embodiment, the base K₃PO₄.H₂O is used in combination with s-Phos as the ligand and Pd(OAc)₂ as a pre-catalyst. The quantity of the base which is used can be any quantity which allows for the formation of the 2-substituted indole compound. In one embodiment, the base is present in about 5 equivalents relative to the ortho-gem-dihalovinylaniline starting material. In another embodiment, the base is K₃PO4 with KOH and is present in about 1.5 equiv. of K₃PO4 and 1.5 equiv. of KOH relative to the or ortho-gem-dihalovinylaniline starting material.

Any solvent may be used in the processes of the present invention for the formation of 2-substituted indoles provided that it does not interfere with the formation of the 2-substituted indole product. Both protic and aprotic and combinations thereof are acceptable. A suitable solvent includes but is not limited to toluene, dioxane, benzene, THF, and the like.

In general, the reagents may be mixed together or added together in any order for the preparation of 2-substituted indoles. Air can be removed from the reaction vessel during the course of the reaction and the solvent and reaction mixtures can be sparged with a non-reactive gas.

The process conditions for the preparation of 2-substituted indoles can be any operable conditions which yield the desired 2-substituted indole product. A preferred temperature for the processes for the production of 2-substituted indoles is about 90° C., although this temperature can be higher or lower depending upon the reagents, reaction conditions and the solvent used. Typical reaction times are between 2 and 14 hours, although longer or shorter times may be used if necessary.

The 2-substituted indole product can be recovered by conventional methods known to those skilled in the art, for example crystallization and silica gel chromatography. The yield of the product 2-substituted indole will vary depending upon the specific pre-catalyst, ligand, base, starting materials and process conditions used. Typically, the 2-substituted indole in provided in a yield greater than 50%, preferably in a yield of greater than 70%, more preferably in a yield greater than 80%. In a preferred embodiment, the s-Phos is present at about 2 mol %, Pd(OAc)₂ is present at about 1 mol %, the base comprises K₃PO₄.H₂O and is present at about 5 equivalents, the solvent is toluene, the ortho-gem-dihalovinylaniline comprises ortho-gem-dibromovinylaniline which is described in Example 1a, and the organoboronic reagent comprises an organoboronic acid of structure R₄—B(OH)₂, and the yield is greater than 60%, preferably greater than 70%, more preferably greater than 80%.

In another embodiment of the present invention, when R₂ is benzyl, or a substituted benzyl in the final 2-substituted indole prepared by the processes of the present invention, the process may also include an additional step of cleavage of the optionally substituted N-benzyl group to afford a 2-substituted indole wherein R₂ is H. Methods and reaction conditions for the cleavage of benzyl groups are known to those skilled in the art, for example, such as those disclosed in Theodora W. Greene, Protective Groups in Organic Synthesis, Wiley Interscience Publications, John Wiley & Sons, New York, copyright 1981), the details of which are incorporated herein by reference. In one embodiment, a mixture of Pd—C, HCOOH and methanol are used for effective cleavage. In another embodiment, H₂/Pd—C is used to afford cleavage. In yet another embodiment, Na/NH₃ can be used to afford cleavage.

The present invention also provides novel processes for the chemical synthesis of the precursor ortho-gem-dibromovinylaniline compounds which are exemplified in the Examples below, for use in the novel process for the chemical synthesis of 2-substituted indole compounds.

In one embodiment of the invention is provided a process for the preparation of an ortho-gem-dibromovinylaniline compound of formula (V) 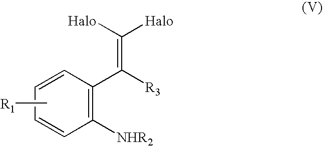
wherein R₁ is independently selected from H; fluoro; alkyl, such as methyl, ethyl, propyl, n-butyl, t-butyl, and the like; alkenyl, and alkynyl; lower alkoxy, aryloxy, haloalkyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₂ is H and R₃ is H or CF₃ or alkynyl optionally substituted at one or more positions with one or more suitable substituents and Halo comprises bromo; said process comprising reacting a nitrobenzaldehyde compound of formula (VI) 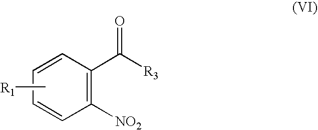
wherein R₁ and R₃ are as defined above for formula (V); with CBr₄ and PPh₃ under conditions effective to generate in situ the olefin ortho-gem-dihalovinyl compound of formula (VII) 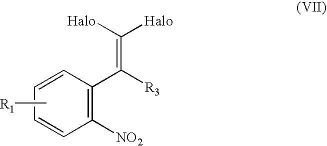
wherein R₁ and R₃ are as defined above for formula (V), and Halo is bromo; followed by reducing the compound of formula (VII) under conditions effective to reduce the nitro group of the compound of formula (VII) to its amino form without affecting the functional groups present in the compound, to afford the compound of formula (V) where R₂ is H and R₃ is H, CF₃, or alkynyl optionally substituted at one or more positions with one or more suitable substituents. Use of this process for the preparation of the ortho-gemhalovinylaniline starting compounds obviates the need for protection of the amino group of the aniline moiety (for a report of the related reaction using CHCl₃ for preparation of the related ortho-gem-dichlorovinyl aniline compound, see Olah et al., J. Org. Chem. Vol. 40, No. 8, 1107, 1975).

Additional methods for the preparation of ortho-gem-dibromovinyl compounds are known in the art, for example, see, Eymery, F.; Iorga, B, Synthesis, 2000, 185-213.

In one embodiment, the starting material aniline comprising ortho-gemdibromovinylaniline as shown in Scheme 24 is obtained from the olefination of 2-nitrobenzaldehyde by treating it with CBr₄/PPh₃ (92%) followed by SnCl₂.2H₂O (90%) reduction in ethanol. Relatively large scale preparation following this method can allow for a one-pot synthesis without isolation of the intermediate, in approximately 85% yield. 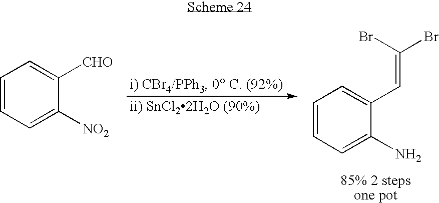
Other reducing conditions for the preparation of ortho-gem-dihalovinylanilines include Fe/HOAc, Fe/catalytic FeCl₃/HOAc/EtOH Zn/NH₄Cl, and hydrogenation with platinum on charcoal doped with vanadium (Degussa F4 (Strem catalogue 2004-2006 78-1512)), illustrated in Scheme 25 and Schemes 28-30. It will be apparent to those skilled in the art that reducing conditions are selected such that they do not affect the functional groups present in the compound. Appropriate conditions can be found in Richard C. Larock, Comprehensive Organic Transformation, Wiley VCH, New York, copyright 1999, the details of which are incorporated herein by reference.

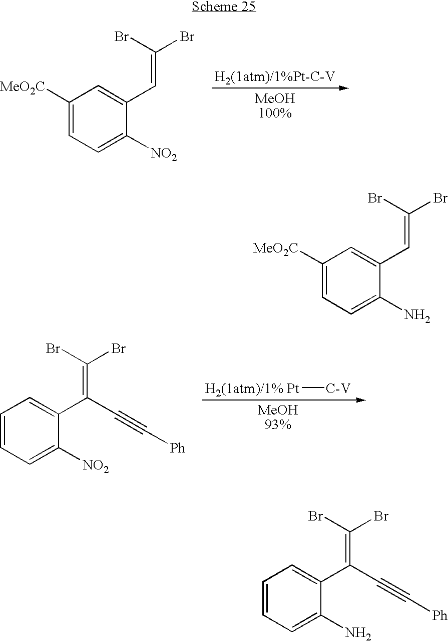

Any solvents may be used in the processes of the present invention for the formation of the starting material ortho-gembromovinylaniline compounds provided that they do not interfere with the formation of the desired ortho-gem-dibromovinylaniline products. Both protic and aprotic and combinations thereof are acceptable. Suitable solvents include but are not limited to dichloromethane and ethanol, ether, dichloromethane, ethyl acetate, THF and the like which are compatible with the reaction.

In general, the reagents in the olefination step may be mixed together or added together in any order. Likewise, reagents in the reduction step of the process mixed together or added together in any order. Air is removed from the reaction vessel during the course of the reaction, and the solvent and reaction mixtures can be sparged with a non-reactive gas.

The process conditions can be any operable conditions which yield the desired ortho-gem-dibromovinylaniline products. A preferred temperature for the processes for the olefination step in production of ortho-gem-dibromovinylaniline products is about 1-5° C., followed by ambient temperature, while a preferred temperature for the reduction step is at the reflux temperature of the solvent employed. Typical reaction times are between 3 and 6 hours, although longer or shorter times may be used if necessary.

The ortho-gem-dihalovinylaniline compounds can be recovered by conventional methods known to those skilled in the art, for example crystallization, silica gel chromatography, vacuum distillation and the like, where appropriate. The yield of the ortho-gem-dihalovinylaniline compounds will vary including depending upon the bases, starting materials and process conditions used. Typically, the ortho-gem-dihalovinylaniline is provided in a yield greater than about 40%. In another embodiment, the ortho-gem-dihalogenvinylaniline compound is afforded in yield of between about 40% and about 85% yield.

In another embodiment of the present invention, the ortho-gem-dihalovinylaniline precursor bears an R₃ substituent other than H or CF₃ or alkynyl. In one embodiment in order to prepare such precursors, the invention provides a process for the preparation of an ortho-gem-dihalovinylaniline compound of formula (V) 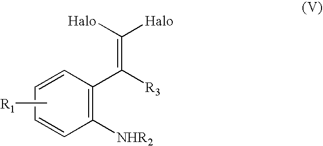
wherein each of the one or more R₁ substituents is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₂ comprises H; R₃ comprises alkyl, haloalkyl, alkenyl, alkynyl, aryl, heteroaryl, cycloalkyl, heterocycle, aryl-(C₁₋₆)alkyl-, or heteroaryl-loweralkyl-, all of which are optionally substituted at one or more substitutable positions with one or more suitable substituents; and Halo comprises bromo or chloro (preferably bromo), the process comprises the steps of: (a) converting a ketone of formula (VIII) 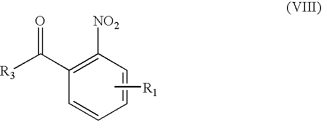
wherein R₁ and R₃ are as defined above for formula (V) into its corresponding olefin derivative of formula (IX) under conditions effective to generate the corresponding olefin derivative of formula (IX) 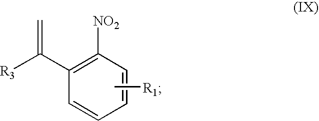
(b) halogenating the olefin derivative of formula (IX) under conditions effective to generate the diahalogen compound of formula (X) 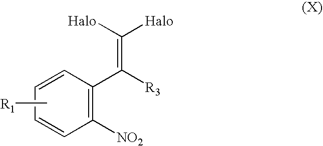
wherein R₁, Halo, and R₃ are defined above; and (c) reducing the compound of formula (X) under conditions effective to reduce the nitro group of the compound of formula (X), without affecting the functional groups present in the compound, to afford the compound of formula (V).

In one embodiment, the ortho-gem-dibromovinylaniline compound of formula (V) is prepared according to Scheme 26 as follows (yields are provide for specific intermediates and final product as indicated to further exemplify the present method):

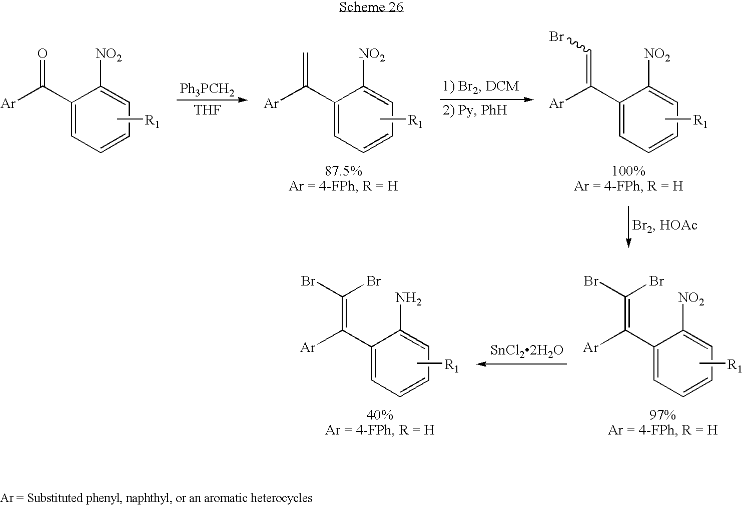

In an alternative embodiment, the ortho-gem-dihalovinylaniline compound may be prepared according to Scheme 27 as follows, which shows the preparation of the ortho-gem-dibromovinylaniline compound of item 15 in Table 2 below according to this method:

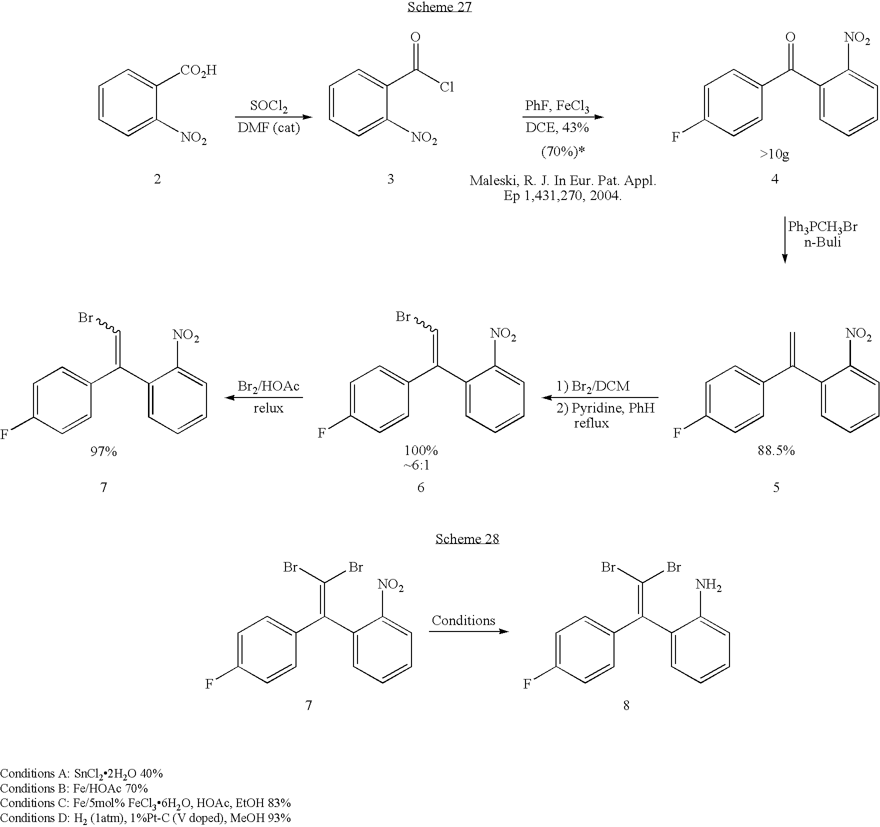

Conditions effecting the reduction of the nitro group to the amino group in the presence of the gem-dihalovinyl functional group include the use of SnCl₂.2H₂O, Fe, or hydrogenation catalyzed by 1% platinum on charcoal doped with vanadium, as shown above.

Selective hydrogenation reaction using 1% platinum on activated carbon doped with vanadium (50% wetted powder; Degussa F4 (Strem catalogue 2004-2006 #78-1512)) is a preferred process as the workup procedure is simple, environmentally benign, and the reaction proceeds with high efficiency, as shown in Scheme 29. Both gem-dibromovinylnitrobenzenes and gem-dichlorovinylnitrobenzenes work well in this process (the former is also illustrated by the examples in Scheme 25).

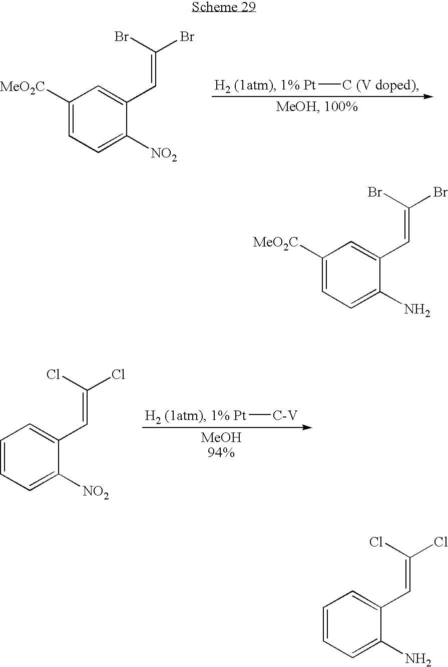

In the case where the nitro group is more sterically hindered, the preferred reduction conditions involve the use of Fe (Crich, D.; Rumthao, S. 2004, 60, 1513-1516) and a catalytic amount of FeCl₃.6H₂O, with HOAc using EtOH as solvent (as per Scheme 30, below).

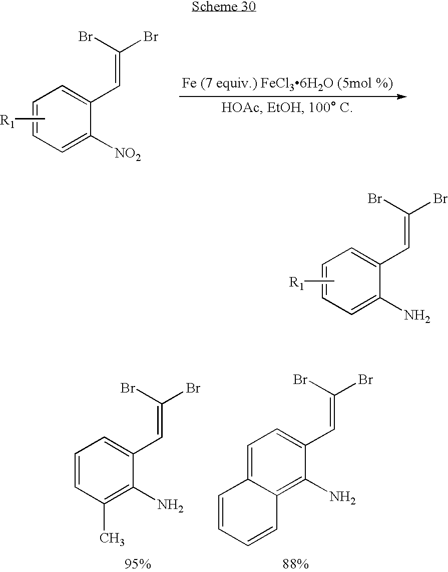

Other alternative embodiments under alternative conditions to effect the olefination other than via the Wittig reaction as shown in Scheme 27, and the elimination/halogenation steps, followed by reduction of the nitro group to an amino group to afford the desired ortho-gem-dihalovinylaniline will likewise be apparent to those skilled in the art. For example, such alternative conditions can be found in Richard C. Larock, Comprehensive Organic Transformation, Wiley VCH, New York, copyright 1999, the details of which are incorporated herein by reference.

For example, an alternative method for the preparation of intermediate compound 5 in Scheme 27 (Nishinaga et al., J. Org. Chem. Vol. 51, 2257, 1986) above is shown in Scheme 31 as follows:

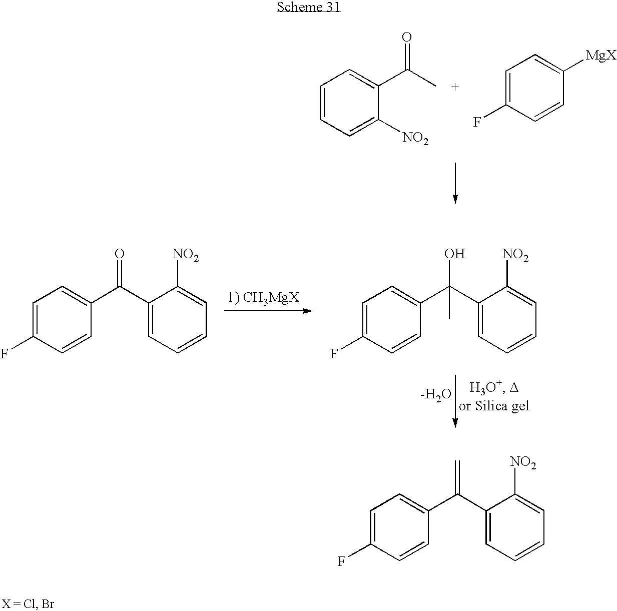

Likewise, another embodiment for the preparation of the intermediate compound 6 from compound 4 of Scheme 27 involves the reaction of compound 4 of Scheme 27 with the Wittig Reagent CHBrPPh₃ (Romero et al, Tetrahedron Lett., Vol 35, 4711, 1994) as shown in Scheme 32 below:

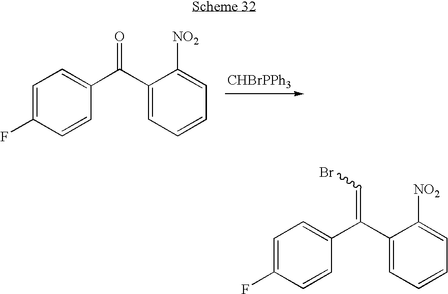

The process conditions for the above embodiment can be any operable conditions which yield the desired ortho-gem-dibromovinylaniline products and their precursors (Richard C. Larock, Comprehensive Organic Transformation, Wiley VCH, New York, copyright 1999).

Any solvent may be used in the processes of the present invention for the formation of the ortho-gem-dihalovinylaniline compounds from ketones provided that it does not interfere with the formation of the ortho-gem-dihalovinylaniline product. Suitable solvents includes but are not limited to those as set out in the examples below.

In general, the reagents may be mixed together or added together in any order for the preparation of the ortho-gem-dihalovinylaniline compounds from ketones provided that it does not interfere with the formation of the ortho-gem-dihalovinylaniline product.

The process conditions for the preparation of the ortho-gem-dihalovinylaniline compounds from either their respective aldehydes or ketones can be any operable conditions which yield the desired the ortho-gem-dihalovinylaniline products. Preferred temperatures for the processes for the production of the ortho-gem-dihalovinylaniline compounds are set out in the examples below, although temperatures can be higher or lower depending upon the reagents, reaction conditions and the solvent used. Typical reaction times are set out in the examples below, although longer or shorter times may be used if necessary. The ortho-gem-dihalovinylaniline compounds can be recovered by conventional methods known to those skilled in the art, for example crystallization and silica gel chromatography.

In another embodiment of present invention is provided a process for the preparation of an ortho-gem-dihalovinylaniline compound of formula (V) 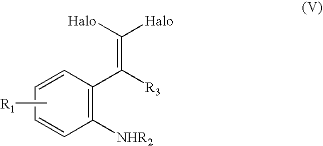
wherein each of the one or more R₁ substituents is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₂ is H, R₃ is H, alkyl, or alkynyl optionally substituted at one or more positions with one or more suitable substituents, and Halo comprises chloro, said process comprising the steps of: (a) reacting a nitrobenzaldehyde or ketone compound of formula (VI) 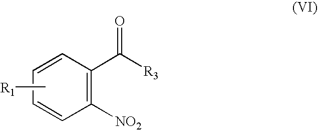
wherein R₁ and R₃ are as defined above for formula (V), with 2 or more equivalents of CHCl₃ and PPh₃ in the presence of 2 or more equivalents of KO^{t}Bu (all equivalents relative to the starting material of formula (VI)) under conditions effective to generate in situ the ortho-gem-dichlorovinyl compound of formula (VII) 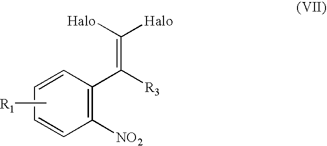
wherein R₁ and R₃ are as defined above and Halo is chloro; and (b) reducing the compound of formula (VII) under conditions effective to reduce the nitro group of the compound of formula (VII), without affecting the functional groups present in the compound, to afford the compound of formula (V). In a preferred embodiment, the reducing agent is SnCl₂.2H₂O (except where R₃ is alknyl). Use of two or more equivalents of CHCl₃ and PPh₃ in the presence of 2 or more equivalents of KOtBu surprisingly and unexpectedly affords higher yields than reported previously (Olah et al., J. Org. Chem. 1975, 40, 8, 1107-1110).
In embodiments where the final 2-substituted indole is N-aryl substituted at the 1-position of the indole, preparation of the N-arylaniline compounds of formula (XI) 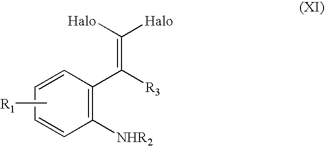
wherein Halo comprises Br, Cl, or I; R₂ comprises aryl which is optionally substituted at one or more substitutable positions with one or more suitable substituents; R₃ comprises H, alkyl, haloalkyl, alkenyl, alkynyl, aryl, heteroaryl, cycloalkyl, heterocycle, aryl-(C₁₋₆)alkyl-, or heteroaryl-loweralkyl-, all of which are optionally substituted at one or more substitutable positions with one or more suitable substituents; and each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (XI); all of which are optionally substituted with one or more suitable substituents at one or more substitutable positions; are made in one embodiment by a process comprising the steps of: reacting a compound of formula (V) 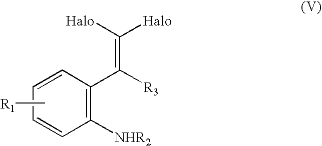
wherein Halo, R₁, R₃ are as defined in Formula (XI) above and R₂ is H, with an organoboron reagent comprising a boronic acid, boronic acid anhydride or BF₃⁻ salt of R₂ in the presence of at least about 1, more preferably at least about 1.5 equivalents of a copper (II) catalyst (relative to the compound of formula (V)), at least about 0.3 equivalents of a C₈-C₂₀ fatty acid, preferably myristic acid (relative to the compound of formula (V)), molecular oxygen, and a non-nucleophilic base, such as lutidine or collidine, at a reaction temperature of between about 40° C. and 60° C., under conditions effective to form a C—N bond between formula (V) and the R₂ group of the organoboron reagent, to afford the N-arylaniline compounds of formula (XI). These compounds are useful for the synthesis of 2-substituted indoles of the present invention as described herein. This improved method is shown in the examples below to be effective for affording arylation of sterically hindered anilines, which can be challenging to achieve by conventional methods, and affords the desired N-arylanilines in good yield with less copper (II) catalyst required than previously known in the art (Antilla et al., Organic Letters 2001, 3, 13, 2077-2079).

In one embodiment, when the 2-substituted indoles comprise N-alkylaniline compounds, of formula (XI) 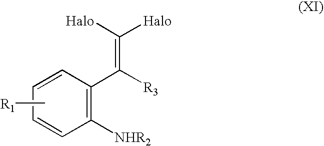
wherein Halo comprises Br, Cl, or I; R₂ comprises alkyl which is optionally substituted at one or more substitutable positions with one or more suitable substituents; R₃ comprises H, alkyl, alkenyl, alkynyl, aryl, heteroaryl, cycloalkyl, heterocycle, aryl-(C₁₋₆)alkyl-, or heteroaryl-loweralkyl-, all of which are optionally substituted at one or more substitutable positions with one or more suitable substituents; and each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (XI); all of which are optionally substituted with one or more suitable substituents at one or more substitutable positions; the process comprises the steps of:
reacting a compound of formula (V) 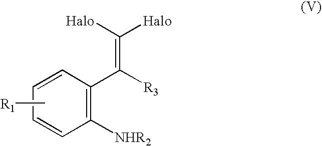
wherein Halo, R₁, R₃ are as defined in Formula (XI) above and R₂ is H, with a suitable alkylating agent, such as alkyl iodide or bromide, under conditions effective to form a C—N bond between formula (V) and the alkyl group of the alkyl halide, to afford the N-alkylaniline compounds of formula (XI). These compounds are useful for the synthesis of 2-substituted indoles of the present invention as described herein.

The preparation of N-alkylated ortho-gem-dihalovinylaniline compounds via standard S_{N}2 substitution reactions is illustrated in Scheme 33: 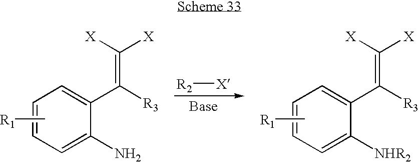
wherein R₁, R₂, R₃, and X are as previously defined for Formula (XI) above, and X′=Br, I. Such reactions are generally carried out in polar aprotic solvents, such as DMSO, DMF, and the like, in the presence of a base, such as K₂CO₃. Catalysts, such as Bu₄NI, may also be used if alkyl bromides are used. Reactions conditions for standard S_{N}2 substitution reactions are well known to those skilled in the art, and it is understood that conditions used to effect such reactions must be compatible with the functional groups present on the substrates. The process conditions for the above embodiment can be any operable conditions which yield the desired N-alkylated products (Richard C. Larock, Comprehensive Organic Transformation, Wiley VCH, New York, copyright 1999).

N-alkylated ortho-gem-dihalovinylaniline compounds may also be prepared via reductive amination reactions, representative examples of which are illustrated in Scheme 34 below, as well as in Scheme 36: 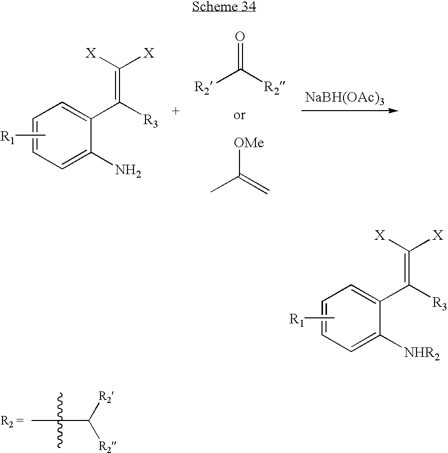
wherein R₁, R₂, R₃, and X are as previously defined for Formula (XI) above. The aldehyde/ketone substituents R₂′ and R₂″ may independently be H, alkyl, aryl, heteroaryl, alkenyl, alkynyl, or other suitable substituents. The reductive sources for such reactions include, but are not limited to, NaBH(OAc)₃, NaBH₄, Na(CN)BH₃, and the like. Standard reductive amination reaction conditions are known to the person skilled in the art, and it is understood that conditions used to effect such reactions must be compatible with the functional groups present on the substrates. The process conditions for the above embodiment can be any operable conditions which yield the desired N-alkylated products (Richard C. Larock, Comprehensive Organic Transformation, Wiley VCH, New York, copyright 1999; Reddy, T. J. et al. Synlett, 2005, 583; Abdel-Magid, A. F. et al J. Org. Chem. 1996, 61, 3849; Bomann, M. D. et al. J. Org. Chem. 1995, 60, 5995).

N-alkylated ortho-gem-dihalovinylaniline compounds may also be prepared via amide reduction reactions, a representative example of which is illustrated in Scheme 35 below: 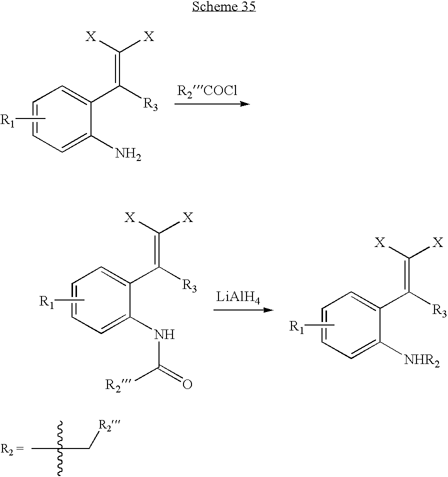
wherein R₁, R₃, and X are as previously defined for Formula (XI) above. The acid chloride substituent R′″ shown in Scheme 35 above may be alkyl, aryl, heteroaryl, alkenyl, alkynyl, or other suitable substituent. N-alkylated ortho-gem-dihalovinylaniline derivatives are obtained by preparing amide derivatives as illustrated in Scheme 35 above, and subsequent reduction of the amide compounds to the desired N-alkylated products. Reagents used to prepare the amide derivative are not limited to acid chlorides, as will be apparent to those skilled in the art, but can also be chosen from carboxylic anhydrides, mixed anhydrides, and like reagents, Likewise, reducing agents for the second reaction step noted above are not limited to LiAlH₄; other reducing agents may be used, so long as the reaction conditions are compatible with the other functional groups present on the molecule. Conditions for the formation of the amide derivatives and their subsequent reduction to the desired N-alkylated products can be any operable conditions which yield the desired compounds, and such conditions can be found in Richard C. Larock, Comprehensive Organic Transformation, Wiley VCH, New York, copyright 1999, the details of which are incorporated herein by reference.

In yet another embodiment of the present invention, the compound 8 of Scheme 28 is useful for the synthesis of the 2-substituted indole, Fluvastatin sodium, as shown in Scheme 36 as follows:

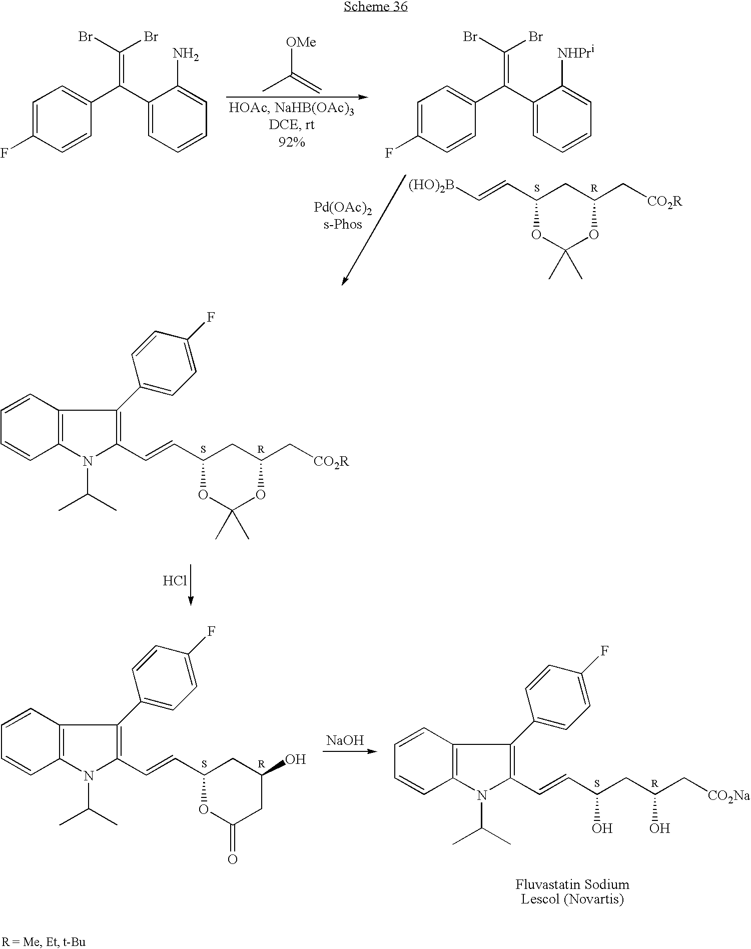

Reductive amination of 2-[2,2-dibromo-1-(4-fluoro-phenyl)-vinyl]-phenylamine with 2-methoxypropene and NaHB(OAc)₃ (Reddy, T. J. et al. Synlett, 2005, 583) afforded the isopropyl substituted aniline derivative, {2-[2,2-Dibromo-1-(4-fluoro-phenyl)-vinyl]-phenyl}isopropylamine, in high yield. This dibromovinyl aniline compound then subsequently couples with the boronic acid fragment noted in Scheme 36 above, to yield the indole compounds (6-{2-[3-(4-fluorophenyl)-1-isoproyl-1H-indole-2-yl]-vinyl}-2,2-dimethyl-[1,3]dioxan-4-yl)acetic acid alkyl esters).

Upon treatment with HCl, the cyclic acetal protecting group falls off and a 6-membered lactone forms 6-{2-[3-(4-fluorophenyl)-1-isoproyl-1H-indole-2-yl]-vinyl}-4-hydroxytetrahydropyran-2-one. This known lactone (Repi\check{c}, O. et al Org. Proc. Res. Dev. 2001, 5, 519) reacts with NaOH to give the pharmacologically active enantiomer (3R,5S) of Fluvastatin Sodium.

According to Scheme 37 shown below, an enantiopure boronic acid 2-(6-alkoxycarbonylmethyl-2,2-dimethyl-[1,3]dioxan-4-yl)ethenylboronic acid, is prepared using standard methods (Miyaura, N.; Suzuki, A. Chem. Rev. 1995, 95, 2457) from an enantiopure acetylene 6-ethynyl-2,2-dimethyl-[1,3]dioxan-4-yl)acetic acid alkyl esters known in the prior art (Miyachi, N. et al Tetrahedron Lett. 1993, 34, 8267). 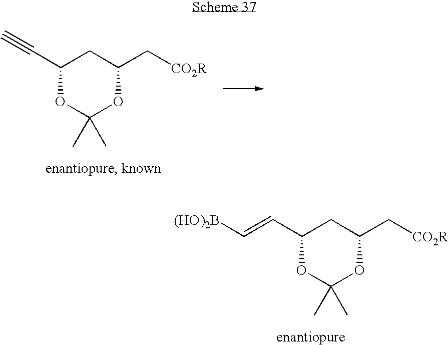
It will be apparent to those skilled in the art that the racemic form of fluvastatin may be obtained by the use of a racemic mixture of the boronic acid in the synthesis illustrated in Scheme 36, as opposed to the enantiopure form of the boronic acid shown above in Scheme 37.

In yet another aspect of the present invention, novel 2-substituted indole compounds and their salts are prepared by the processes of the present invention, including each of the following 2-substituted indoles and their salts:

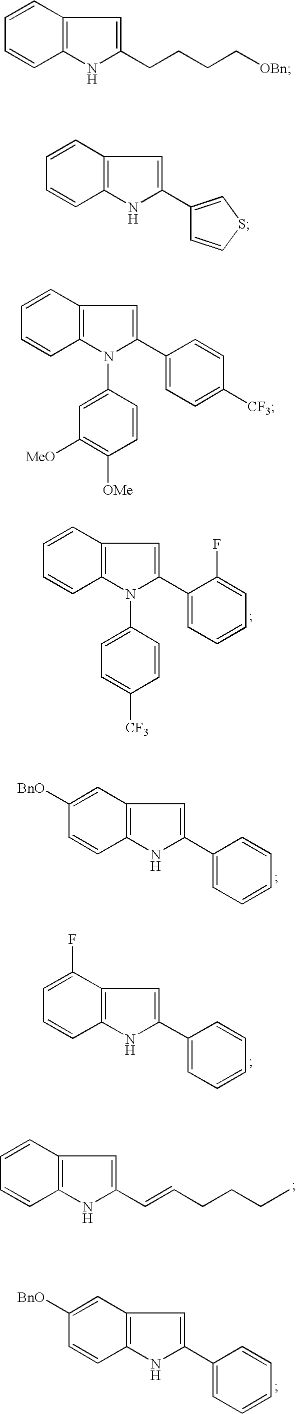

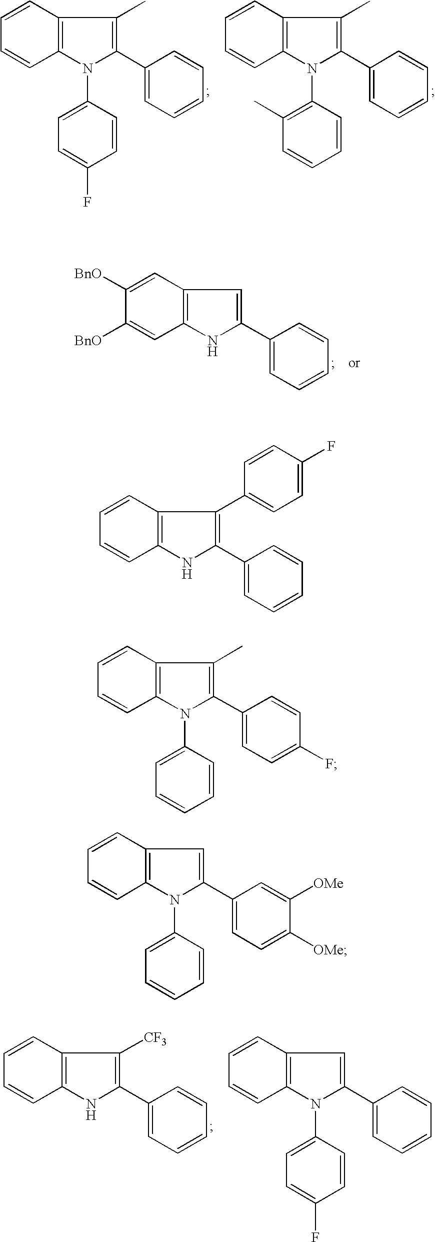

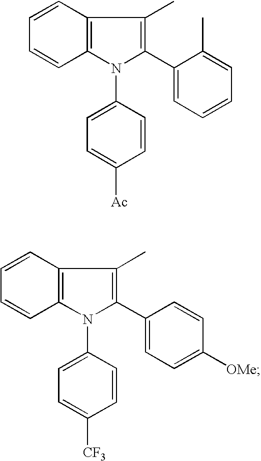

Compounds of similar structure such as those contained in Canadian Patent No. 1,081,237 and U.S. Pat. No. 4,522,808 have been shown to be useful as sunscreen compounds and for the protection of photosensitive dyestuffs since they absorb UV radiation. The present novel 2-substituted indole compounds are similar in structure and use of these compounds for the absorption of UV radiation, in particular as sunscreen compounds is envisaged.

In yet another embodiment are novel ortho-gem-dihalogenvinylaniline compounds prepared by the process of the present invention, including the following compounds or their salts, which are useful in the preparation of the desired 2-substituted indole compounds:

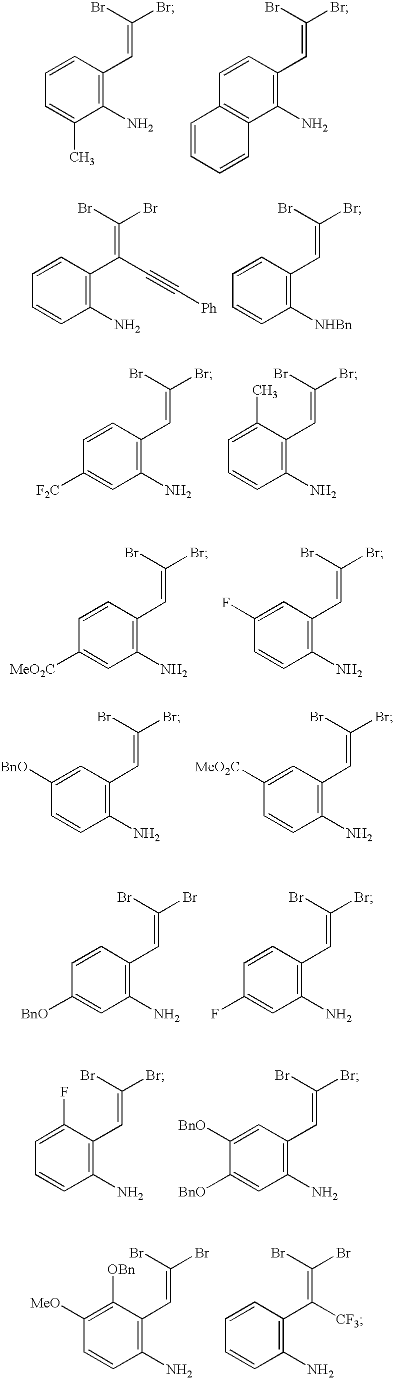

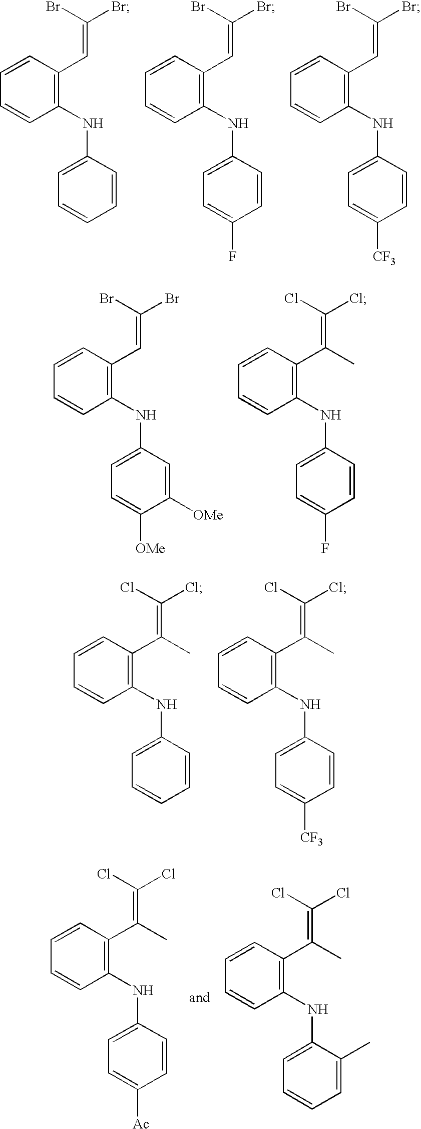

The results of various tandem C—N and C—C bond formation reactions to afford 2-substituted indoles in good yield using various aryl and heteroarylboronic acids of different electronic and steric character and 2-(2,2-dibromo-vinyl)-phenylamine are shown in as shown in Table 1 below (Table 1, entries 1-10). Using an alkenyl boronic acid and alkenyl catechol boronic ester (Table 1, entries 11-13) also gave the desired indole product in good yield.

One of the merits of the Suzuki coupling reaction is its ability to couple sp²-sp³ carbons (for a review see: Chemler, S. R.; Trauner, D.; Danishefsky, S. J. Angew. Chem., Int. Ed. 2001, 40, 4544-4568). Subjecting commercially available triethylboron and alkyl 9-BBN reagents (prepared in situ by premixing a terminal alkene and 9-BBN overnight at 20° C.) to the reaction conditions (60° C. in THF) afforded the desired indole products in good yield (Table 1, entries 14-16).

TABLE 1

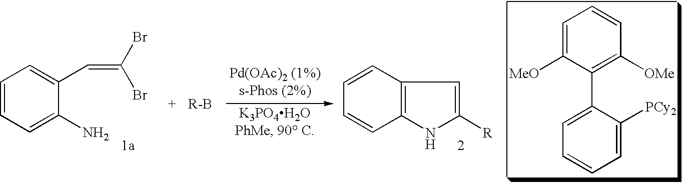

| | | | Pre- | | | |
|---|---|---|---|---|---|---|
| | | | Catalyst | | Reaction | |
| | Boronic acid, | | loading | | time | Yield |
| Entry | Alkyl BBN | Indoles | (%) | Example | (h) | (%) |
| 1 | 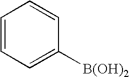 | 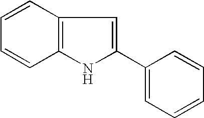 | 1 | 2a | 6 | 84 |
| 2 | 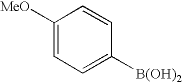 | 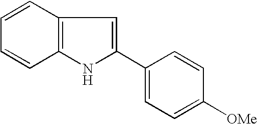 | 1 | 2b | 2 | 83 |
| 3 | 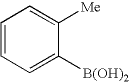 | 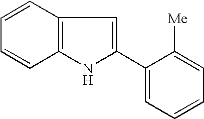 | 1 | 2c | 4 | 82 |
| 4 | 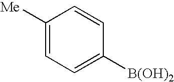 | 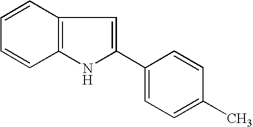 | 1 | 2d | 5 | 88 |
| 5 | 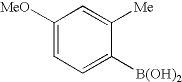 | 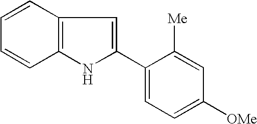 | 1 | 2e | 5.5 | 79 |
| 6 | 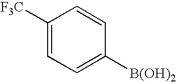 | 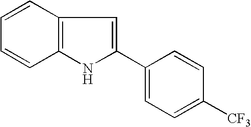 | 1 | 2f | 7 | 75 |
| 7 | 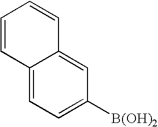 | 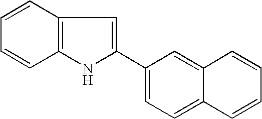 | 1 | 2g | 7 | 82 |
| 8 | 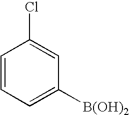 | 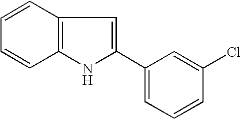 | 3.3 | 2h | 2.5 | 60 |
| 9 | 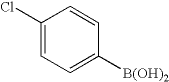 | 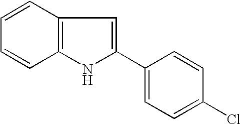 | 3.3 | 2i | 2.5 | 57 |
| 10 | 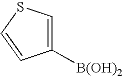 | 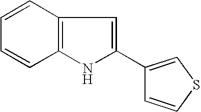 | 2 | 2j | 12 | 86 |
| 11 | 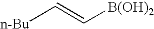 | 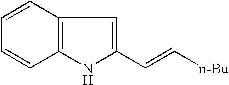 | 2 | 2k | 5 | 80 |
| 12 | 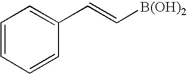 | 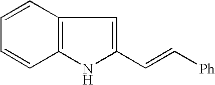 | 2 | 2l | 7 | 68 |
| 13 | 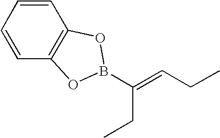 | 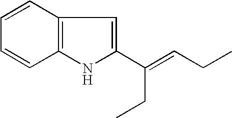 | 3 | 2m | 6 | 73 |
| 14 | Et₃B | 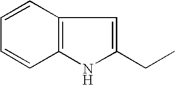 | 2 | 2n | 2 | 77 |
| 15 | 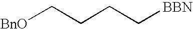 | 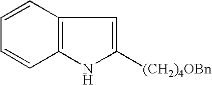 | 2 | 2o | 4 | 77 |
| 16 | n-HexBBN | 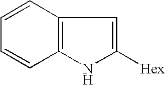 | 2 | 2p | 3 | 79 |

The reaction conditions as shown in Table 1 above can tolerate a wide variety of organoboron reagents. The effect of substitution on the aniline nitrogen of the ortho-gem-dihalovinylaniline starting material are shown in Table 2 below. Use of the N-benzyl protected secondary amine worked almost as well as its non-protected version. In contrast, the use of electron-withdrawing and activating acetyl or tosyl protecting groups on the nitrogen group gave low yields under optimized conditions. Use of the non-protected aniline also afforded flexibility in the starting materials that could be employed in the reactions, and thus, the scope of 2-substituted anilines that could be made by the present processes in good yields, and by way of a simplified protocol. While initial results gave a good yield of 75% using N-acetyl-2-gem-dibromovinylaniline, Pd₂(dba)₃/P(o-tol)₃ and K₂CO₃, the scope of the boronic acids was found to be limited and yields were generally resistant to further optimization. However, comparable yields for a variety of boronic acids could be obtained using the free amino group. For example, Pd(OAc)₂ coupled with the s-Phos ligand in the presence of K₃PO₄.H₂O in toluene (90° C.) gave 2-phenylindole in good yield (84%) with an attractively low pre-catalyst loading of 1%.

In Table 2, various substituted ortho-gem-dibromovinylanilines were reacted with phenylboronic acid under the noted reaction conditions. This methodology proved to be a very general and efficient method to prepare several functionalized indoles (Table 2, entries 2-9, 11-15, and 17-19). In particular, preparation of 2- and 4-substituted indoles (entry 2-3), which are generally regarded as challenging targets, were prepared from their corresponding anilines in good yield despite their longer reaction times. In general, electronic factors had little effect on yield with the exception of extremely electron-rich substrates (entry 8), which gave slightly lower yields presumably due to substrate instability. Use of ortho-gem-dichlorovinylaniline derivatives (entries 10 and 16) also gave near quantitative yield of the expected indoles. Entries 14-17 also show that the present methods work with ortho-gem-dihalovinylanilines bearing an R₃ substituent other than H. For example, when R₃=alkyl, fluoroalkyl, aryl, and alkynyl (entries 14-17), the tandem coupling reaction proceeded smoothly to afford the desired product in good to excellent yield.

TABLE 2

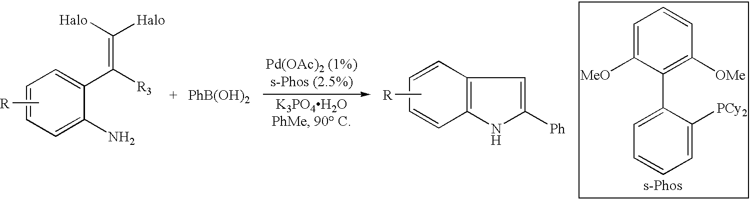

| | | | Pre- | | | |
|---|---|---|---|---|---|---|
| | | | Cat. | | | |
| | | | loading | | Time | Yield |
| Entry | Substrate | Indoles | (%) | Example | (h) | (%) |
| 1 | 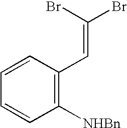 | 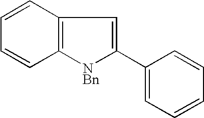 | 1 | 2q | 4 | 82 |
| 2 | 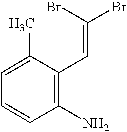 | 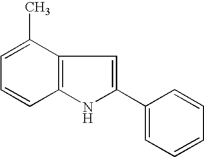 | 5 | 2r | 2 | 77 |
| 3 | 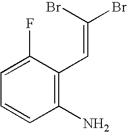 | 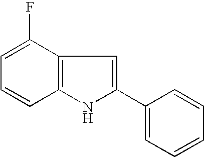 | 1 | 2s | 14 | 88 |
| 4 | 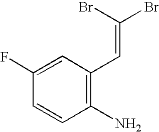 | 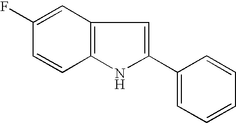 | 1 | 2t | 2 | 87 |
| 5 | 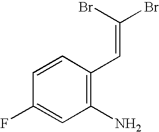 | 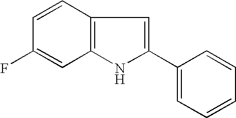 | 2 | 2u | 2.5 | 80 |
| 6 | 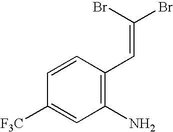 | 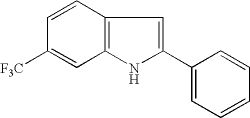 | 1 | 2v | 2.5 | 90 |
| 7 | 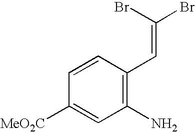 | 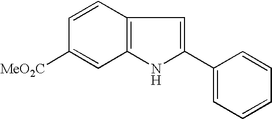 | 1 | 2w | 8.5 | 90 |
| 8 | 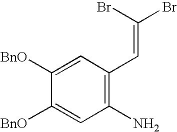 | 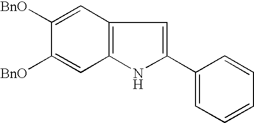 | 3.3 | 2x | 4.5 | 57 |
| 9 | 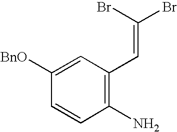 | 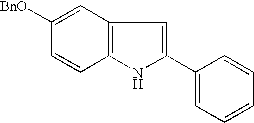 | 2 | 2y | 3 | 86 |
| 10 | 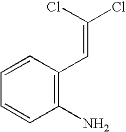 | 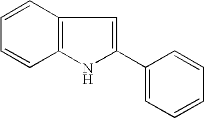 | 5 | 2z | 2 | 95^{b} |
| 11 | 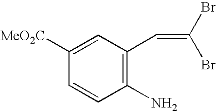 | 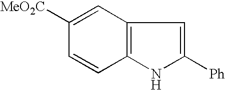 | 3 | 2aa | 2.5 | 87 |
| 12 | 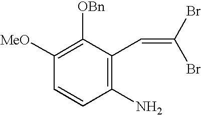 | 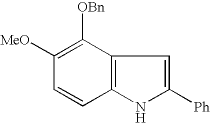 | 3 | 2bb | 2 | 72 |
| 13 | 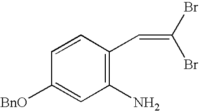 | 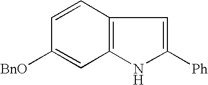 | 3 | 2cc | 2.5 | 72 |
| 14 | 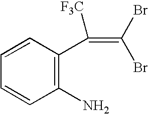 | 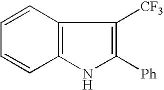 | 3 | 2dd | 1 | 79 |
| 15 | 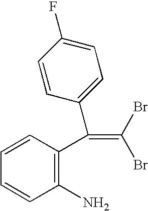 | 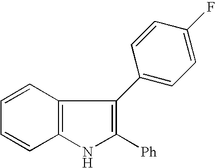 | 3 | 2ee | 2.5 | 90 |
| 16 | 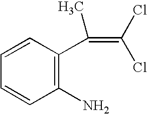 | 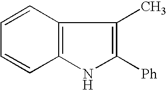 | 3 | 2ff | 2 | 96 |
| 17 | 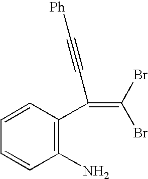 | 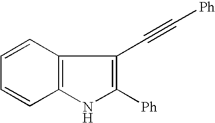 | 3 | 2gg | 1.5 | 77 |
| 18 | 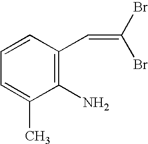 | 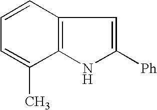 | 3 | 2hh | 2 | 89% |
| 19 | 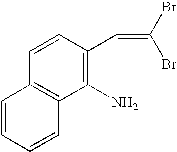 | 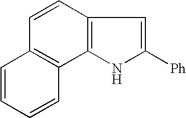 | 3 | 2ii | 2 | 77% |

While wishing not to be bound by any particular theory, experimental evidence suggests that the reaction for the 2-substituted indoles wherein the 3-position is substituted with H, does not go solely through a typical Pd-catalyzed C—N coupling reaction (see Scheme 38, below). Use of a deuterium labelled ortho-gem-dibromovinylaniline 4 under standard reaction conditions gave the expected indole product with 16% deuterium leaching. Control experiments show no proton exchange on 3-H for 2-phenylindole under these reaction conditions. Since it is well established that the trans-bromo is much more prone to oxidative addition, this suggests that for 2-substituted indoles where the 3-position is substituted with H, the vinylpalladium intermediate (5) undergoes β-hydride elimination to give the bromoalkyne intermediate 7 and DPd(II)Br 6 (Shen, W.; Wang, L. J. Org. Chem. 1999, 64, 8873-8879). Pd(II)-mediated 5-endo-dig cyclization ((a) Rudisill, D. E.; Stille, J. K. J. Org. Chem. 1989, 54, 5856-5866; (b) Taylor, E. C.; Katz, A. H.; Salgado-Zamora, H.; McKillop, A. Tetrahedron Lett. 1985, 26, 5963-5966) gives the 2-bromoindole 9 which subsequently undergoes Suzuki coupling with phenylboronic acid to give the desired 2-phenylindole in near quantitative yield. Proton exchange of 6 with its environment is thought to be responsible for the observed deuterium leaching ((a) Kudo, K.; Hidai, M.; Murayama, T.; Uchida, Y. J. Chem. Soc., Chem. Commun. 1970, 1701-1702. (b) Leoni, P.; Sommovigo, M.; Pasquali, M.; Midollini, S.; Braga, D.; Sabatino, P. Organometallics 1991, 10, 1038-1044).

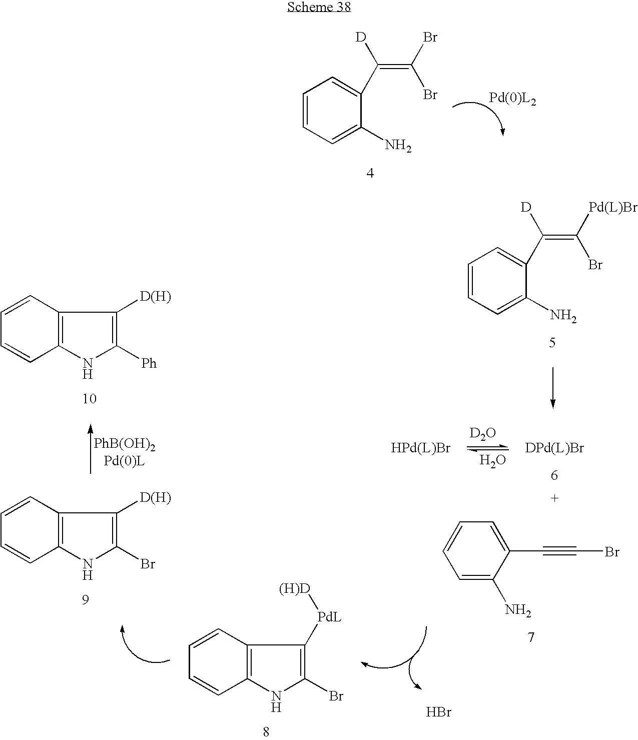

In order to further evidence the versatility of this method to prepare various different 2-substituted indole compounds, including 1,2-substituted indoles, in Table 3 below, ortho-gem-dibromovinylaniline 1a was reacted with various arylboronic acids to prepare various N-aryl ortho-gem-dibromovinylaniline compounds, which in turn were reacted with various arylboronic acids to afford various 1,2-diarylindole compounds in good yields as indicated below. The various N-aryl ortho-gem-dibromovinylaniline compounds were prepared by the novel copper-mediated processes described herein for the coupling of aniline and aryl boronic acids.

Various 1,2-diarylindoles are known in the art as being biologically active molecules, thereby evidencing the further utility of the present processes for the preparation of various 2-substituted indoles. Potential applications of 1,2-diarylindoles include their use as COX-2 inhibitors (Gungor, T.; Teulon, J.-M. In PCT Int. Appl.; (Laboratoires UPSA, Fr.). WO 98 05639, 1998, p 59), as estrogen agonists and antagonists (Von Angerer, E.; Strohmeier, J. J. Med. Chem. 1987, 30, 131-136; Biberger, C.; Von Angerer, E. J. Steroid Biochem. Mol. Bio. 1998, 64, 277-285), and as organic electroluminescent devices (Lin, T.-s. In U.S. Pat. No. 6,790,539, 2004.)

TABLE 3

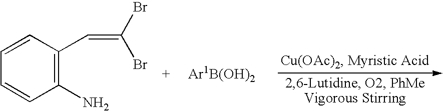

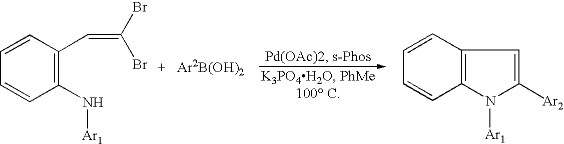

| Entry | Ar¹B(OH)₂/Yield (%) | Ar²B(OH)₂ | Indoles | Yield (%) |
|---|---|---|---|---|
| 1 | PhB(OH)₂89% | PhB(OH)₂ | 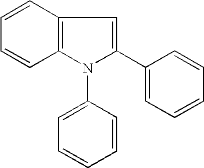 | 92 |
| 2 | PhB(OH)₂89% | 4-FPhB(OH)₂ | 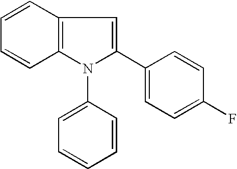 | 86 |
| 3 | PhB(OH)₂89% | 3,4-(OMe)₂PhB(OH)₂ | 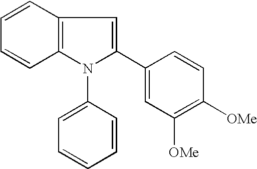 | 60 |
| 4 | 4-FPhB(OH)₂72% | PhB(OH)₂ | 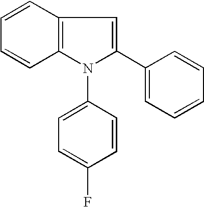 | 90 |
| 5 | 4-CF₃PhB(OH)₂84% | 2-FPhB(OH)₂ | 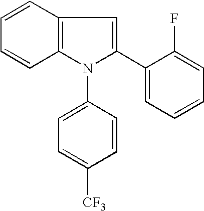 | 82 |
| 6 | 3,4-(OMe)₂PhB(OH)₂56% | 4-CF₃PhB(OH)₂ | 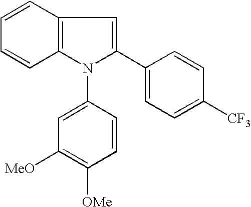 | 81 |

Further evidence of the versatility of the present methods is provided in Table 4 below, wherein ortho-gem-dichlorovinylaniline 1q having an R₃ methyl group was reacted with various boronic acids to afford various N-arylanilines, which in turn were reacted with various arylboronic acids to afford the 1,2,3-substituted indoles in good yield. Likewise, Table 4 illustrates the versatility of the novel copper-mediated C—N coupling reactions between the ortho-gem-dihalovinylaniline compounds and various arylboronic acid compounds.

TABLE 4

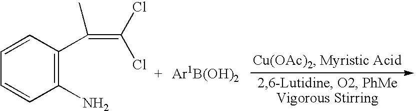

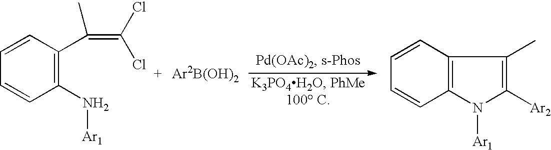

| Entry | Ar¹B(OH)₂/Yield (%) | Ar²B(OH)₂ | Indoles | 2^{nd} step Yield (%) |
|---|---|---|---|---|
| 1 | PhB(OH)₂98% | 4-FPhB(OH)₂ | 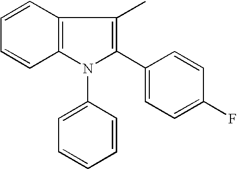 | 96 |
| 2 | 4-FPhB(OH)₂65% | PhB(OH)₂ | 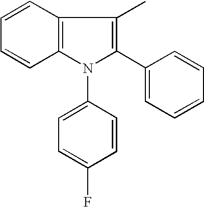 | 94 |
| 3 | 4-CF₃PhB(OH)₂69% | 4-MeOPhB(OH)₂ | 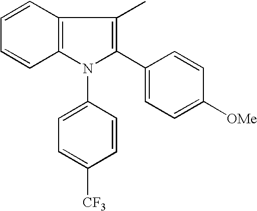 | 79 |
| 4 | 4-AcPhB(OH)₂70% | 2-MePhB(OH)₂ | 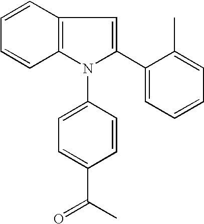 | 75 |
| 5 | 2-MePhB(OH)₂70% | PhB(OH)₂ | 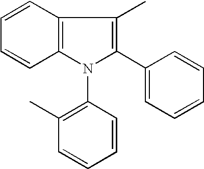 | 77 |

In one embodiment, the processes of the invention are utilized in the preparation of the KDR kinase inhibitor 3-[5-[[4-(methylsulfonyl)-1-piperazinyl]methyl]-1H-indole-2-yl]quinolin-2(1H)-one:

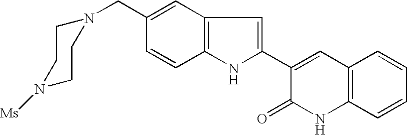

The KDR inhibitor shown above can be synthesized starting from commercially available methyl 3-formyl-4-nitrobenzoate (Scheme 39). The whole process takes seven steps and provides the desired product in an overall 64.7% yield. Ortholithiation of 2-methoxyquinoline followed by trapping with B(OPrⁱ)₃ gave 2-methoxyquinolinylboronic acid (5a) in 95% yield. The boronic acid 5a was then used to effect the tandem coupling reaction with 1u to afford Compound 5b. Compound 5b was coverted into Compound 5e in three steps. Compound 5e is known to convert into the final compound, 3-[5-[[4-(methylsulfonyl)-1-piperazinyl]methyl]-1H-indole-2-yl]quinolin-2(1H)-one, in 98% yield (Wong, A. et. al. J. Org. Chem. 2004, 69, 7761-7764); thus, the overall yield of this sequence would be 64.7%. This is higher than the prior art procedures, which result in overall yields of 55-60% of the desired product.

It will be apparent to a person skilled in the art that alternate conditions may be used to effect the transformations from compounds 5b to 5e as illustrated in Scheme 39. For example, such alternative conditions can be found in Richard C. Larock, Comprehensive Organic Transformation, Wiley VCH, New York, copyright 1999, the details of which are incorporated herein by reference.

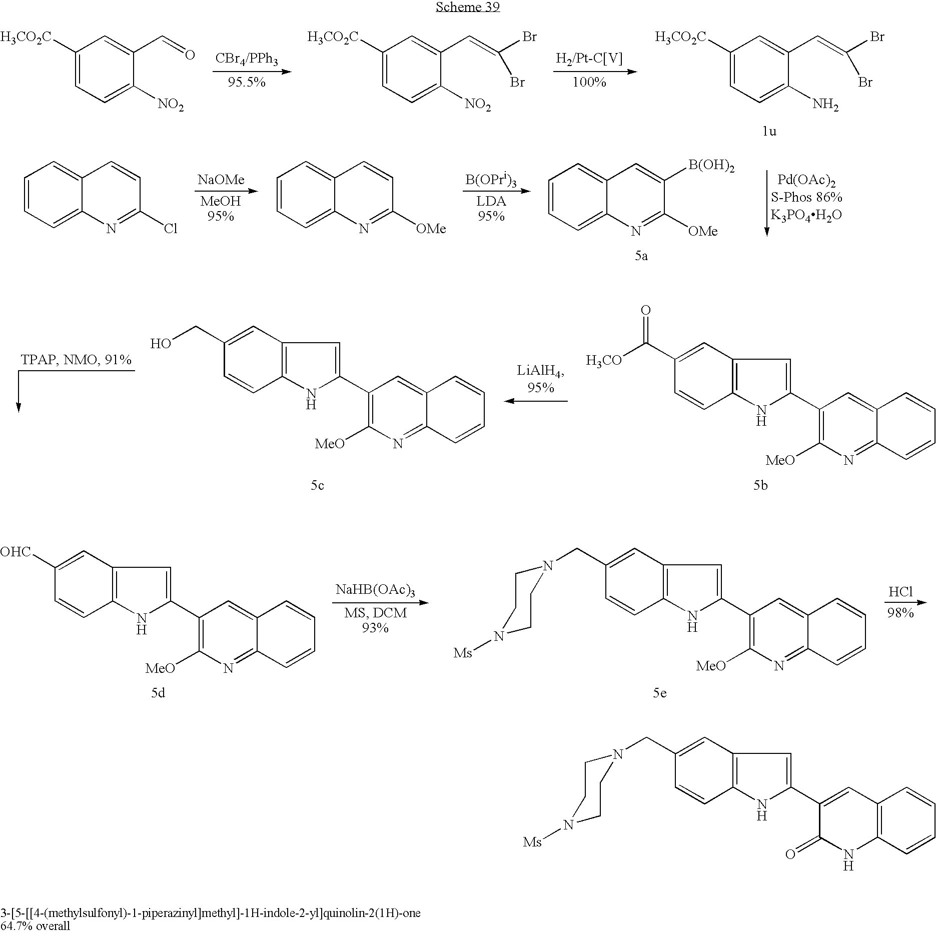

Detailed procedures for the formation of the precursor vinylaniline compounds and their use in reactions are set forth in the Examples section below. Likewise, detailed procedures for the formation of various 2-substituted indoles are set forth in the examples below. The following examples are intended to illustrate, but in no way limit the scope of the present invention.

### EXAMPLES

General Procedures: All reactions were carried out under N₂. Solvents and solutions were added with a syringe, unless otherwise noted. Analytical TLC was performed using EM separations precoated silica gel 0.2 mm layer UV fluorescent sheets. Column chromatography was carried out as “flash chromatography” as reported by Still using Merck 60 (230-400 mesh) silica gel (Still, W. C.; Kahn, M.; Mitra, A. J. Org. Chem. 1978, 43, 2923-5). Unless otherwise specified, extracts were dried over MgSO₄ and solvents were removed with a rotary evaporator at aspirator pressure.

Toluene was distilled under N₂ from Na/benzophenone immediately prior to use. s-Phos was purchased from Strem Chemical Company and other pre-catalysts or reagents were obtained from commercial sources without further purification.

Melting points were taken on a Fisher-Johns melting point apparatus without correction. IR spectra were obtained using Nicolet DX FT IR spectrometer as thin films on NaCl plates. High-resolution mass spectra were obtained from a VG 70-250S (double focusing) mass spectrometer at 70 eV. ¹H, ¹³C, and ¹⁹F NMR spectra were obtained using Varian Mercury 400, Mercury 300 or Gemini 300 spectrometers. ¹H spectra were referenced to tetramethylsilane (TMS, 0 ppm) using CDCl₃ as solvent, DMSO-D₅ residue peaks (2.50 ppm) using DMSO-d₆; ¹³C spectra were referenced to solvent carbons (77.23 ppm for CDCl₃; 39.57 ppm for DMSO-d₆). When carbons are equivalent, no special notation is used.

Preparation of Ortho-Gem-Dihalovinylaniline Compounds

The results of the preparation of various ortho-gem-dibromovinylanilines of Tables 1 and 2 above are shown in Examples 1a-1p below.

### Example 1a

### General Procedure for the One-Pot Synthesis of 2-gem-dibromovinylanilines—Preparation of 2-(2,2-Dibromo-vinyl)-phenylamine

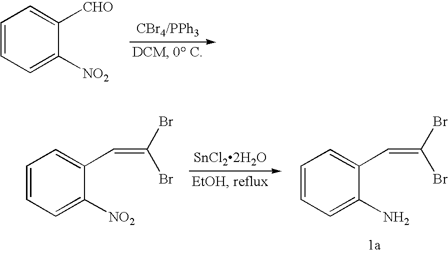

To a solution of 2-nitrobenzaldehyde (9.07 g, 60 mmol) and CBr₄ (29.8 g, 90 mmol) in DCM (300 mL) at 0° C. was added dropwise a solution of PPh₃ (47.2 g, 180 mmol)) in DCM (200 mL) by an addition funnel. The addition rate was controlled so that the internal temperature was at 1-5° C. After addition (˜1 h), the mixture was stirred for another 0.5 h before warmed to rt and stirred for another 1 h. The reaction mixture was filtered through a short plug of silica gel (120 g) and the silica gel was washed with copious amount of DCM until no product was found. Solvent was removed under vacuum to give a solid mixture of the desired product and triphenylphosphine oxide. The mixture (˜50 g) was added absolute EtOH (200 mL) and SnCl₂.H₂O (67.7 g, 300 mmol). The suspension was heated to 100° C. (reflux) under N₂ for 45 min. The mixture was cooled to rt and most solvent was removed under vacuum. H₂O (150 mL) and EtOAc (150 mL) were added and the mixture was added carefully solid K₂CO₃ until PH>10. EtOAc layer was separated from the heterogeneous mixture and the aqueous phase was extracted with EtOAc until it is free of product (5×100 mL). The combined organic solution was washed with brine and dried over Na₂SO₄/K₂CO₃. Solvent was removed under vacuum and the residue was redissolved in Et₂O. Precipitated Ph₃PO was removed by filtration. The product was purified by flash chromatography on silica gel eluted with 10% EtOAc in hexanes. The product was obtained as an oily compound which was solidified under high vacuum overnight or upon frozen for days (14.2 g, 85% over 2 steps). mp 40-42° C. ¹H NMR (300 MHz, CDCl₃) δ 7.33 (1H, s), 7.30 (1H, d, J=7.7 Hz), 7.16 (1H, ddd, J=7.7, 7.7, 1.4 Hz), 6.78 (1H, t, J=7.6 Hz), 6.70 (1H, dd, J=8.0, 0.8 Hz), 3.70 (2H, br). ¹³C NMR (75 MHz, CDCl₃) δ 143.8, 134.3, 129.9, 129.4, 122.0, 118.6, 116.0, 93.0 (Topolski, M. J. Org. Chem. 1995, 60, 5588-5594).

### Example 1b

### Preparation of 2-(2,2-Dibromo-vinyl)-3-methyl-phenylamine

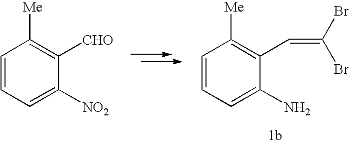

The general procedure of Example 1A was followed starting from 2-methyl-6-nitro-benzaldehyde (Harvey, I. W.; Smith, D. M.; White, C. R. J. Chem. Soc., Perkin 1 1996, 1699-1703) (6 mmol scale). The product was purified by flash chromatography (5% EtOAc in hexanes) to afford 1.21 g (69% over 2 steps). R_{f}=0.21 (5% EtOAc in hexanes). mp 42-43° C. IR (neat, cm⁻¹) 3461 (m), 3377 (m), 2986 (w), 1612 (s), 1579 (m), 1467 (s), 1302 (m). ¹H NMR (400 MHz, CDCl₃) δ 7.30 (1H, s), 7.06 (1H, t, J=7.8 Hz), 6.62 (1H, d, J=7.5 Hz), 6.56 (1H, d, J=7.9 Hz), 3.69 (2H, br), 2.20 (3H, s). ¹³C NMR (100 MHz, CDCl₃) δ 143.4, 137.0, 135.3, 129.3, 122.1, 120.2, 113.2, 94.9, 20.2. HRMS (ESI) calc'd for C₉H₁₀Br₂N ([MH]⁺): 289.9174. Found: 289.9161.

### Example 1c

### Synthesis of 2-(2,2-Dibromo-vinyl)-3-fluoro-phenylamine

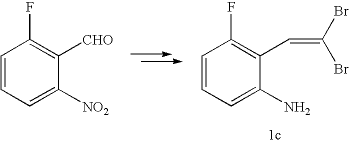

The general procedure of Example 1A was followed, starting from 2-fluoro-6-nitrobenzaldehyde (6.5 mmol scale). The product was purified by flash chromatography (10% EtOAc in hexanes) to afford 1.56 g (81% over 2 steps). R_{f}=0.20 (10% EtOAc in hexanes) as a semi-solid. IR (neat, cm⁻¹) 3479 (m), 3391 (s), 1626 (s), 1579 (s), 1464 (s), 1318 (m), 1243 (s), 1116 (m), 1056 (m). ¹H NMR (400 MHz, CDCl₃) δ 7.20 (1H, s), 7.10 (1H, dddd, J=8.2, 8.2, 6.4, 0.7 Hz), 7.49-6.45 (2H, m), 3.85 (2H, br). ¹³C NMR (100 MHz, CDCl₃) δ 160.1 (J_{CF}=246 Hz), 145.3 (J_{CF}=5.7 Hz), 130.6 (J_{CF}=10.5 Hz), 129.4 (J_{CF}=1.5 Hz), 111.1 (J_{CF}=2.7 Hz), 110.5 (J_{CF}=19.5 Hz), 105.2 (J_{CF}=22.1 Hz), 96.7 (J_{CF}=1.7 Hz). ¹⁹F NMR (376 MHz, CDCl₃) δ −112.2 (1F, t, J_{FH}=8.4 Hz). HRMS (ESI) calc'd for C₈H₇NFBr₂ ([MH]⁺) 293.8923. Found: 293.8919.

### Example 1d

### Synthesis of 4-Benzyloxy-2-(2,2-dibromo-vinyl)-phenylamine

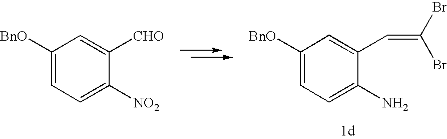

The general procedure of Example 1A was followed starting from 5-benzyloxy-2-nitro-benzaldehyde (Astles, P. C.; Brown, T. J.; Halley, F.; Handscombe, C. M.; Harris, N. V.; McCarthy, C.; McLay, I. M.; Lockey, P.; Majid, T.; Porter, B.; Roach, A. G.; Smith, C.; Walsh, R. J. Med. Chem. 1998, 41, 2745-2753) (7.0 mmol scale). The product was purified by flash chromatography (10% EtOAc in hexanes) to afford 2.04 g (76% over 2 steps) as a white solid. R_{f}=0.15 (10% EtOAc in hexanes). mp 67-68° C. IR (neat, cm⁻¹) 3423 (w), 3353 (w), 1603 (s) 1499 (s), 1426 (m), 1260 (s), 1229 (m), 1001 (s). ¹H NMR (300 MHz, CDCl₃) δ 7.43-7.26 (6H, m), 6.97 (1H, d, J=2.7 Hz), 6.84 (1H, dd, J=8.8, 3.0 Hz), 6.64 (1H, d, J=8.8 Hz), 5.00 (3H, s), 3.44 (2H, br). ¹³C NMR (75 MHz, CDCl₃) δ 151.6, 137.9, 137.4, 134.0, 128.7, 128.1, 127.7, 122.8, 117.7, 117.4, 115.3, 92.9, 71.0. HRMS (ESI) calcd for C₁₅H₁₄NOBr₂ ([MH]⁺) 381.9436. Found: 381.9425.

### Example 1e

### Synthesis of 2-(2,2-Dibromo-vinyl)-5-fluoro-phenylamine

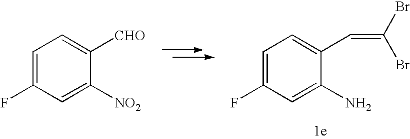

The general procedure of Example 1A was followed starting from 4-fluoro-2-nitrobenzaldehyde (Kalir, A. Org. Synth. 1966, 46, 81-84) (10 mmol scale). The product was purified by flash chromatography (10% EtOAc in hexanes) to afford 2.35 g (80% over 2 steps) as a solid. R_{f}=0.19 (10% EtOAc in hexanes). mp 72-73° C. IR (neat, cm⁻¹) 3464 (w), 3382 (m), 1621 (s), 1494 (m), 1434 (m), 1300 (w), 1168 (m), 1114 (w). ¹H NMR (400 MHz, CDCl₃) δ 7.26-7.22 (2H, m, overlap), 6.48 (1H, ddd, J=8.5, 8.5, 2.6 Hz), 6.40 (1H, dd, J=10.4, 2.6 Hz), 3.81 (2H, br). ¹³C NMR (100 MHz, CDCl₃) δ 163.8 (J_{CF}=244 Hz), 145.6 (J_{CF}=11.4 Hz), 133.3, 131.0 (J_{CF}=9.9 Hz), 117.8 (J_{CF}=2.3 Hz), 105.6 (J_{CF}=22.0 Hz), 102.5 (J_{CF}=25.1 Hz), 93.5 (2.2). ¹⁹F NMR (282 MHz, CDCl₃) δ −111.7 (1F, dd, J_{FH}=16, 9.2 Hz). Anal. Calc'd for C₈H₆Br₂NF: C, 32.58; H, 2.05; N, 4.75. Found: C, 32.86; H, 2.20; N, 4.78.

### Example 1f

### Synthesis of 2-(2,2-Dibromo-vinyl)-5-trifluoromethyl-phenylamine

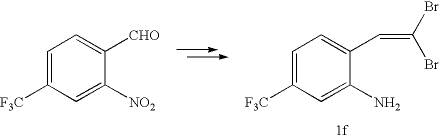

The general procedure of Example 1A was followed starting from 2-nitro-4-trifluoromethyl-benzaldehyde (Lewandowska, E.; Kinastowski, S.; Wnuk, S. F. Can. J. Chem. 2002, 80, 192-199) (11.6 mmol scale). The product was purified by flash chromatography (5→10% EtOAc in hexanes) to afford 3.10 g (80% over 2 steps) as an oil. R_{f}=0.27 (10% EtOAc in hexanes). IR (neat, cm⁻¹) 3486 (w), 3397 (m), 1627 (s), 1436 (s), 1338 (s), 1252 (m), 1168 (s), 1124 (s). ¹H NMR (300 MHz, CDCl₃) δ 7.37 (1H, d, J=8.1 Hz), 7.31 (1H, s), 7.00 (1H, d, J=8.1 Hz), 6.93 (1H, s), 3.88 (2H, br). ¹³C NMR (100 MHz, CDCl₃) δ 144.1, 133.1, 131.8 (q, J_{CF}=32.2 Hz), 130.0, 124.8 (q, J_{CF}=1.3 Hz), 124.1 (q, J_{CF}=271 Hz), 115.0 (q, J_{CF}=3.8 Hz), 112.4 (J_{CF}=3.9 Hz), 94.9 (q, J_{CF}=0.8 Hz). ¹⁹F NMR (282 MHz, CDCl₃) δ −63.0. HRMS (ESI) calc'd for C₉H₇NF₃Br₂ ([MH]⁺) 343.8891. Found: 343.8907.

### Example 1g

### Synthesis of 4,5-Bis-benzyloxy-2-(2,2-dibromo-vinyl)-phenylamine

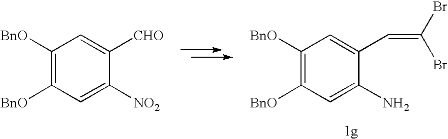

The general procedure of Example 1a was followed starting from 4,5-bis-benzyloxy-2-nitro-benzaldehyde (5 mmol scale). The product was purified by flash chromatography (20% EtOAc in hexanes) to afford 1.01 g (80% over 2 steps) as an off-white solid (1.01 g, 41% in 2 steps). R_{f}=0.21 (20% EtOAc in hexanes). mp 95-98° C. IR (neat, cm⁻¹) 3445 (w), 3372 (m), 1614 (m), 1505 (s), 1454 (m), 1427 (m), 1290 (m), 1213 (s), 1125 (s). ¹H NMR (400 MHz, CDCl₃) δ 7.43-7.24 (10H, m), 7.20 (1H, s), 6.99 (1H, s), 6.28 (1H, s), 5.10 (2H, s), 5.06 (2H, s), 3.44 (2H, br). ¹³C NMR (100 MHz, CDCl₃) δ 151.0, 141.2, 139.4, 137.8, 137.2, 133.3, 128.8, 128.6, 128.1, 127.9, 127.8, 127.4, 117.9, 114.1, 103.1, 91.4, 72.9, 71.1. Anal. Calc'd for C₂₂H₁₉Br₂NO₂: C, 54.01; H, 3.91; N, 2.86. Found: C, 54.31; H, 4.24; N, 2.94.

### Example 1h

### Synthesis of 3-Amino-4-(2,2-dibromo-vinyl)-benzoic acid methyl ester

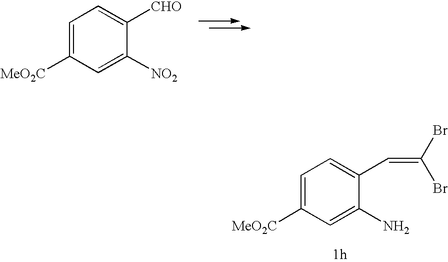

The general procedure of Example 1a was followed starting from 4-formyl-3-nitro-benzoic acid methyl ester (5 mmol scale). The product was purified by flash chromatography (20% EtOAc in hexanes) to afford 1.06 g (80% over 2 steps) as a yellow solid. R_{f}=0.20 (20% EtOAc in hexanes). mp 97-99° C. IR (neat, cm⁻¹) 3469 (w), 3378 (s), 1710 (s), 1631 (s), 1567 (m), 1501 (m), 1433 (s), 1315 (s), 1246 (s), 1111(s). ¹H NMR (300 MHz, CDCl₃) δ 7.45-7.30 (4H, m), 3.90-3.85 (5H, m). ¹³C NMR (75 MHz, CDCl₃) δ 167.0, 143.9, 133.5, 131.3, 129.5, 126.0, 119.5, 116.9, 94.4, 52.4. HRMS (ESI) calc'd for C₁₀H₁₀NO₂Br₂ ([MH]⁺) 333.9072. Found: 333.9089.

### Example 1i

### Synthesis of 2-(2,2-Dibromo-vinyl)-4-fluoro-phenylamine

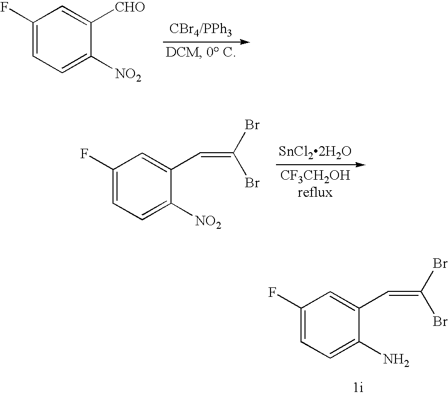

The general procedure of preparing 2-gem-dibromovinylnitrobenzene was followed by starting from 5-fluoro-2-nitro-benzaldehyde 5 (10 mmol scale) to afford the intermediate (3.1 g, 95%) after chromatographic purification (5% EtOAc in hexanes). The nitro intermediate (0.416 g, 1.28 mmol) and SnCl₂.2H₂O (1.45 g, 6.40 mmol) in 1,1,1-trifluoroethanol (7 mL) was reflux under N₂ for 8 h. The mixture was taken into H₂O/Et₂O (20 mL/20 mL) and neutralized with K₂CO₃. After extraction with Et₂O (3×20 mL), the product was purified by flash chromatography (10% EtOAc in hexanes) to afford the product (0.303 g, 80%) as an oil. R_{f}=0.20 (10% EtOAc in hexanes). IR (neat, cm⁻¹) 3453 (m), 3378 (s), 3001 (w), 1626 (s), 1493 (s), 1434 (m), 1260 (m), 1207 (m), 1151 (m). ¹H NMR (300 MHz, CDCl₃) δ 7.29 (1H, s), 7.07 (1H, dd, J=9.4, 2.7 Hz), 6.89 (1H, ddd, J=8.4, 8.4, 3.0 Hz), 6.64 (1H, dd, J=8.8, 4.7 Hz), 3.58 (2H, br). ¹³C NMR (75 MHz, CDCl₃) δ 155.9 (J_{CF}=237 Hz), 140.0 (J_{CF}=2.0 Hz), 133.2 (J_{CF}=1.7 Hz), 122.8 (J_{CF}=7.7 Hz), 117.1 (J_{CF}=8.0 Hz), 116.7 (J_{CF}=22.6 Hz), 115.6 (J_{CF}=23.5 Hz), 93.9. ¹⁹F NMR (282 MHz, CDCl₃) δ −125.8 (1F, ddd, J_{FH}=8.4, 8.4, 4.6 Hz). HRMS (ESI) calc'd for C₈H₇NFBr₂ ([MH]⁺) 293.8923. Found: 293.8923.

### Example 1j

### Synthesis of Benzyl-[2-(2,2-dibromo-vinyl)-phenyl]-amine

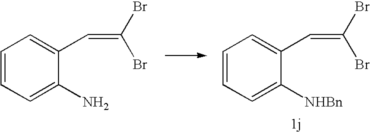

To a suspension of the aniline (1.385 g, 5 mmol) and K₂CO₃ (0.834 g, 6 mmol) in DMF (15 ml) was added BnBr (1.03 g, 6 mmol). The mixture was stirred at rt for 48 h under N₂. Then mixture was diluted with Et₂O (20 mL), washed with H₂O (3×20 mL), brine (15 mL). The mixture was purified by flash chromatography (2.5% EtOAc in hexanes) to afford a white crystalline solid (1.40 g, 76%). mp 93-95° C. IR (neat, cm⁻¹) 3433 (m), 1600 (s), 1576 (m), 1509 (s), 1449 (m), 1324 (s),1247 (m). ¹H NMR (300 MHz, CDCl₃) δ 7.36-7.27 (7H, m), 7.19 (1H, t, J=7.6 Hz), 6.74 (1H, t, J=7.6 Hz), 6.62 (1H, d, J=8.2 Hz), 4.37 (2H, d, J=4.9 Hz), 4.02 (1H, br). ¹³C NMR (75 MHz, CDCl₃) δ 145.0, 139.1, 134.2, 130.1, 129.5, 128.9, 127.5, 121.8, 117.3, 111.3, 93.6, 48.2. HRMS (ESI) calc'd for C₁₅H₁₄BrN ([MH]⁺): 365.9487. Found: 365.9482.

### Example 1k

### Synthesis of 4-Amino-3-(2,2-dibromo-vinyl)-benzoic acid methyl ester

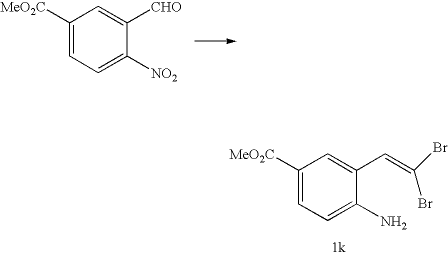

The general procedure of Example 1a was followed starting from 5-formyl-4-nitro-benzoic acid methyl ester (6.5 mmol scale). The product was purified by flash chromatography (20→30% EtOAc in hexanes) to afford 1.917 g (88% over 2 steps) as a yellowish solid. R_{f}=0.20 (20% EtOAc in hexanes). mp 112-113° C. IR (neat, cm⁻¹) 3476 (m), 3368 (s), 3244 (w), 2950 (w), 1698 (s), 1623 (s), 1502 (m), 1437 (s), 1289 (s), 1243 (s), 1198 (s), 1149 (m), 1106 (m). ¹H NMR (400 MHz, CDCl₃) δ 7.97 (1H, d, J=1.8 Hz), 7.84 (1H, dd, J=8.5, 1.9 Hz), 7.29 (1H, s), 6.98 (1H, d, J=8.4 Hz), 4.14 (2H, br), 3.86 (3H, s). ¹³C NMR (75 MHz, CDCl₃) δ 167.0, 147.9, 133.2, 131.8, 131.7, 120.8, 120.0, 114.9, 94.8, 52.0. HRMS calc'd for C₁₀H₉NO₂Br₂ ([M]⁺) 332.9000. Found: 332.9004.

### Example 1l

### Synthesis of 5-Benzyloxy-2-(2,2-dibromo-vinyl)phenylamine

### Step 1: Synthesis of 2-Benzyloxy-3-methoxy-6-nitrobenzaldehyde

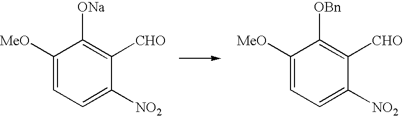

2-Hydroxy-3-methoxy-6-nitrobenzaldehyde, sodium salt was prepared as red solid according literature procedure (Press, J. B.; Bandurco, V. T.; Wong, E. M.; Hajos, Z. G.; Kanojia, R. M.; Mallory, R. A.; Deegan, E. G.; McNally, J. J.; Roberts, J. R.; Cotter, M. L.; Graden, D. W.; Lloyd, J. R. J. Heterocycl. Chem. 1986, 23, 1821-1828). The sodium phenoxide solid (3.89 g, 17.8 mmol) was suspended in a mixed solvent of DMF (20 mL) and CH₃CN (20 mL). K₂CO₃ (0.5 g) and BnBr (3.42 g, 20 mmol) were added and the mixture was heated to 100° C. for 4 h until red colour suspension disappeared. The mixture was cool to rt, added H₂O (50 mL), extracted with DCM and EtOAc. The organic phase was dried over MgSO₄ and solvent was removed under vacuum. The solid was recrystallized from 5% EtOAc in hexanes and washed with small amount of Et₂O to afford a white crystalline solid (5.0 g, 98%). ¹H NMR (300 MHz, CDCl₃) δ 10.2 (1H, s), 7.96 (1H, d, J=9.1 Hz), 7.43-7.32 (5H, m), 7.06 (1H, d, J=9.2 Hz), 5.07 (2H, s), 4.02 (3H, s).

### Step 2: Synthesis of 5-Benzyloxy-2-(2,2-dibromo-vinyl)phenylamine

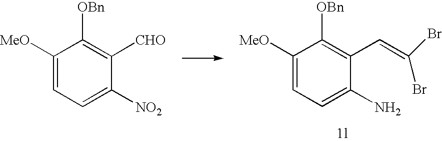

The general procedure of Example 1a was followed starting from 2-Benzyloxy-3-methoxy-6-nitro-benzaldehyde (11.56 mmol scale). The product was purified by flash chromatography (20% EtOAc in hexanes) to afford 3.44 g (72% over 2 steps) a solid. R_{f}=0.16 (20% EtOAc in hexanes). mp 96-97° C. IR (neat, cm⁻¹) 3451 (w), 3366 (m), 2940 (m), 1617 (m), 1487 (s), 1441 (m), 1266 (s), 1126 (m), 1076 (m), 1226(m). ¹H NMR (300 MHz, CDCl₃) δ 7.46-7.28 (5H, m), 7.17 (1H, s), 6.83 (1H, d, J=8.7 Hz), 6.45 (1H, d, J=8.7 Hz), 4.98 (2H, s), 3.82 (3H, s), 3.51 (2H, br). ¹³C NMR (75 MHz, CDCl₃) δ 145.9, 145.6, 138.0, 137.7, 133.0, 128.7, 128.6, 128.2, 118.6, 115.1, 111.2, 94.7, 75.3, 57.0. HRMS calc'd for C₁₆H₁₅NO₂Br₂ ([M]⁺) 410.9470. Found: 410.9470.

### Example 1m

### Synthesis of 5-Benzyloxy-2-(2,2-dibromo-vinyl)-phenylamine

### Step 1: Synthesis of 4-Benzyloxy-2-nitrobenzaldehyde

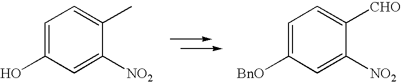

A mixture of 4-methyl-3-nitrophenol (2.53 g, 16.5 mmol), BnBr (2.83 g, 19.8 mmol), K₂CO₃ (2.75 g, 19.8 mmol) and tetrabutylammonium iodide (0.61 g, 1.65 mmol) was stirred at rt under N₂ for 18 h. The mixture was diluted with Et₂O (40 mL), washed with H₂O (20 mL), NaOH (1M, 10 mL), H₂O (20 mL), NaHCO₃ (20 mL) and brine (20 mL) and dried over MgSO₄. The mixture was further purified by flash chromatography on silica gel (10% EtOAc in hexanes) to afford a light yellow solid (4.0 g, 100%). The solid of 4-benzyloxy-2-nitrotoluene was converted into corresponding aldehyde according the literature procedure by reacting with CH(OMe)₂NMe₂ (5.9 g, 49.3 mmol) in DMF (10 mL) at 140° C. for 60 h, followed by oxidation with NaIO₄ (10.5 g, 49.3 mmol) (Vetelino, M. G.; Coe, J. W. Tetrahedron Lett. 1994, 35, 219-222). The product was purified by flash chromatography on silica gel (20% EtOAc in hexanes) to afford a light yellowish solid (3.33 g, 79%). ¹H NMR (300 MHz, CDCl₃) δ 10.3 (1H, s), 7.96 (1H, d, J=8.8 Hz), 7.59 (1H, d, J=2.5 Hz), 7.43-7.27 (5H, m), 7.28 (1H, dd, J=8.6, 2.5 Hz), 5.20 (2H, s).

### Step 2: Synthesis of 5-Benzyloxy-2-(2,2-dibromo-vinyl)-phenylamine

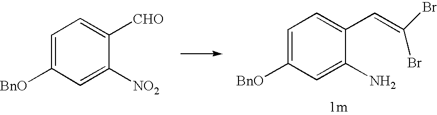

The general procedure of Example 1a was followed starting from 4-Benzyloxy-2-nitro-benzaldehyde (7.5 mmol scale). The product was purified by flash chromatography (10% EtOAc in hexanes) to afford 2.35 g (82% over 2 steps) a light tan solid. R_{f}=0.20 (10% EtOAc in hexanes). mp 93-94° C. IR (neat, cm⁻¹) 3470 (w), 3383 (s), 1615 (s), 1572 (m), 1502 (s), 1300 (s), 1187 (s), 1016 (s). ¹H NMR (400 MHz, CDCl₃) δ 7.42-7.30 (5H, m), 7.25 (2H, m), 6.43 (1H, dd, J=8.7, 2.3 Hz), 6.30 (1H, d, J=2.4 Hz), 5.02 (2H, s), 3.69 (2H, br). ¹³C NMR (100 MHz, CDCl₃) δ 160.3, 145.3, 137.1, 133.7, 130.6, 128.8, 128.2, 127.7, 115.2, 105.4, 102.0, 91.8, 70.1. HRMS (ESI) calc'd for C₁₅H₁₄NOBr₂ ([MH]⁺) 381.9436. Found: 381.9455.

### Example 1n

### Synthesis of 2-(2,2-Dibromo-1-trifluoromethyl-vinyl)-phenylamine

### Step 1: Synthesis of 1-(2,2-Dibromo-1-trifluoromethyl-vinyl)-2-nitro-benzene

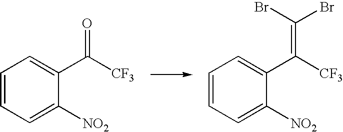

To a solution of 2,2,2-Trifluoro-1-(2-nitro-phenyl)-ethanone (O'Dell, D. K.; Nicholas, K. M. Heterocycles 2004, 63, 373-382) (1.88 g, 8.58 mmol) and CBr₄ in DCM (45 mL) was dropwise added a solution of PPh₃ solution In DCM (45 mL) at 0° C. The mixture was stirred for another 1 h and warmed to rt and continuously stirred for 0.5 h. The mixture was precipitated by addition of Et₂O (20 mL) and hexanes (50 mL), filtered through a short silica gel column. The product was further purified by flash chromatography (10% EtOAc in hexanes) to afford the product as a light yellow solid (2.83 g, 88%). R_{f}=0.24 (10% EtOAc in hexanes). mp 58-59° C. IR (neat, cm⁻¹) 1590 (m), 1532 (s), 1347 (s), 1297 (s), 1197 (s), 1182 (s), 1138 (s). ¹H NMR (400 MHz, CDCl₃) δ 8.26 (1H, dd, J=8.1, 1.3 Hz), 7.76 (1H, ddd, J=7.6, 7.6, 1.3 Hz), 7.67 (1H, ddd, J=7.9, 7.9, 1.5 Hz), 7.40 (1H, dd, J=7.6, 1.4 Hz). ¹³C NMR (100 MHz, CDCl₃) δ 147.0, 135.8 (q, J_{CF}=33.7 Hz), 134.6, 131.6, 131.2, 130.9, 125.5, 121.6 (q, J_{CF}=276 Hz), 101.0 (q, J_{CF}=3.1 Hz). ¹⁹F NMR (376 MHz, CDCl₃) δ −59.2 (s).

### Step 2: Synthesis of 2-(2,2-Dibromo-1-trifluoromethyl-vinyl)-phenylamine

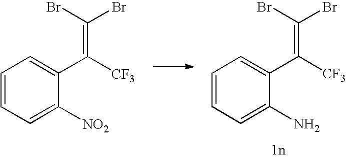

A mixture of 1-(2,2-Dibromo-1-trifluoromethyl-vinyl)-2-nitro-benzene (1.875 g, 5 mmol) and SnCl₂.2H₂O (5.64 g, 25 mmol) in EtOH (30 mL) was reflux under Ar for 8 h. The mixture was taken into EtOAc (50 mL) and neutralized with K₂CO₃. After extraction with Et₂O (3×30 mL), the organic phase was dried over Na₂SO₄. After removal of solvent, the product was purified by flash chromatography (10% EtOAc in hexanes) to afford the product (1.54 g, 89%) as an oil (solidified in freezer). R_{f}=0.25 (10% EtOAc in hexanes). mp 25-26° C. IR (neat, cm⁻¹) 3481 (m), 3393 (s), 3029 (w), 1621 (s), 1578 (s), 1493 (s), 1454 (m), 1292 (s), 1199 (s), 1176 (s), 1130 (s). ¹H NMR (400 MHz, CDCl₃) δ 7.22 (1H, ddd, J=7.8, 7.8, 1.4 Hz), 6.97 (1H, dd, J=7.7, 1.3 Hz), 6.80 (1H, ddd, J=7.6, 7.6, 0.9 Hz), 7.56 (1H, d, J=8.1 Hz), 3.71 (2H, br). ¹³C NMR (100 MHz, CDCl₃) δ 143.9, 135.6 (q, J_{CF}=33.2 Hz), 131.0, 129.6, 122.3 (q, J_{CF}=277 Hz), 121.1, 118.9, 116.3, 103.8 (q, J_{CF}=2.8 Hz). ¹⁹F NMR (376 MHz, CDCl₃) δ −58.8 (s). HRMS calc'd for C₉H₆NF₃Br₂ ([M]⁺) 342.8819. Found: 342.8830.

### Example 1o

### Preparation of 2,2-dibromo-1-(4-fluorophenyl)-1-(2-aminophenyl)ethene

### Step 1: Synthesis of 1-(4-Fluorophenyl)-1-(2-nitrophenyl)ethene

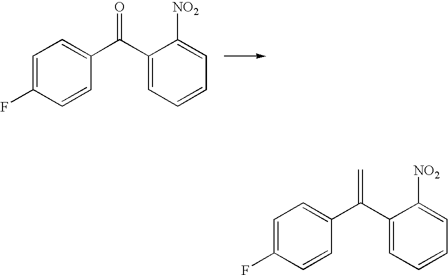

To a suspension of methyltriphenylphosphonium bromide (11.2 g, 31 mmol, Pre-dried at 100° C. under high vacuum of 0.2 nm n Hg) in THF (50 mL) was added dropwise n-BuLi (19.5 mL, 1.6 M in hexane, 31 mmol) at 0° C. After addition, the red/orange solution was stirred at 0° C. for additional 0.5 h. To this Wittig reagent was dropwise added a solution of (4-Fluoro-phenyl)-(2-nitro-phenyl)-methanone (Maleski, R. J. In Eur. Pat. Appl. Ep 1,431,270, 2004) (6.13 g, 25 mmol) in THF (40 mL). The reaction was stirred at 0° C. for another 2 h before it was quenched by NH4Cl (saturated, 30 mL). The mixture was extracted with EtOAc (3×50 mL) and the organic layer was washed with brine, dried over MgSO₄. The residue after removal of solvent under vacuum was purified by column chromatograph (silica gel) using 10% EtOAc in hexanes to afford a slightly yellow solid (5.32 g, 87.5%). R_{f}=0.25 (10% EtOAc in hexanes). mp 45-46° C. IR (neat, cm⁻¹) 3070 (m), 1604 (s), 1528 (s), 1351 (s), 1229 (s), 1160 (s). ¹H NMR (400 MHz, CDCl₃) δ 7.94 (1H, dd, J=8.1, 1.1 Hz), 7.64 (1H, ddd, J=7.6, 7.6, 1.3 Hz), 7.51 (1H, ddd, J=7.8, 7.8, 1.3 Hz), 7.45 (1H, dd, J=7.6, 1.4 Hz), 7.23-7.19 (2H, m), 7.00-6.95 (2H, m), 5.68 (1H, s), 5.29 (1H, s). ¹³C NMR (100 MHz, CDCl₃) δ 162.8 (J_{CF}=248 Hz), 149.0, 145.7, 136.9, 135.5 (J_{CF}=3.8 Hz), 133.1, 132.5, 129.0, 128.5 (J_{CF}=7.7 Hz), 124.6, 115.6, 115.5 (J_{CF}=21.5 Hz). ¹⁹F NMR (376 MHz, CDCl₃) δ −113.8 (1F, dddd, J_{FH}=8.5, 8.5, 5.3, 5.3 Hz). HRMS calc'd for C₁₄H₁₀NO₂F ([M]⁺) 243.0696. Found: 243.0692. Anal. Calc'd for C₁₄H₁₀NO₂F: C, 69.13; H, 4.14; N, 5.76. Found: C, 69.24; H, 4.21; N, 5.72.

### Step 2: Synthesis of 2,2-Dibromo-1-(4-fluorophenyl)-1-(2-nitrophenyl)ethene

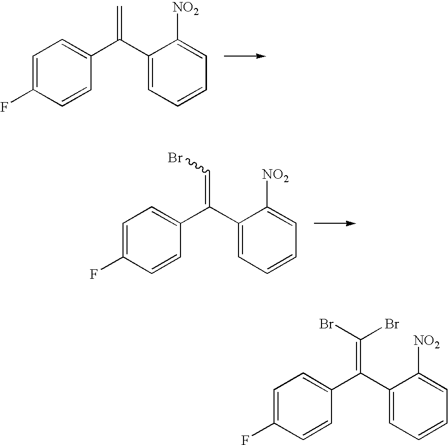

To a solution of 1-(4-fluorophenyl)-1-(2-nitrophenyl)ethene (5.03 g, 20.7 mml) in DCM (30 mL) was dropwise added Br₂ (3.5 g) solution in DCM (5 mL) at 0° C. The mixture was stirred for another 2 h and warmed to rt. Solvent was removed under vacuum to give a solid. The solid was dissolved in benzene (30 mL) and added pyridine (8 mL). The mixture was heated (100° C. oil bath) under reflux for 3 h and cooled to rt, diluted with EtOAc (40 mL), washed with HCl (1 M, 2×25 mL), NaHCO₃ (Saturated, 25 mL), brine (25 mL) and dried over MgSO₄. The solvent was removed under vacuum to give a red-coloured crude intermediate, which is the Z/E mixture of monobrominated alkene (6.66 g, 100%). The solid was taken into acetic acid (60 mL) and added Br₂ (5.5 g). The mixture was heated to 115° C. (under reflux) for 5 h and warm to rt. Excess Br₂ and solvent was removed under vacuum. The residue was taken into NaHCO₃ (Saturated, 50 mL), extracted with Et₂O (2×50 mL). Organic layer was washed with NaHCO₃ (Saturated, 25 mL), brine (25 mL) and dried over MgSO₄. The crude product was purified by flash chromatography on silica gel (10% EtOAc in hexanes) to afford the desired product was a slight-tan solid (8.0 g, 96%). R_{f}=0.21 (7.5% EtOAc in hexanes). mp 99-100° C. IR (neat, cm⁻¹) 3071 (m), 1603 (s), 1527 (s), 1505 (s), 1348 (s), 1232 (s), 1160 (m). ¹H NMR (400 MHz, CDCl₃) δ 8.09 (1H, dd, J=8.2, 1.2 Hz), 7.69 (1H, ddd, J=7.6, 7.6, 1.3 Hz), 7.53 (1H, ddd, J=8.0, 7.7, 1.4 Hz), 7.48 (1H, dd, J=7.7, 1.3 Hz), 7.44-7.40 (2H, m), 7.04-6.98 (2H, m). ¹³C NMR (100 MHz, CDCl₃) δ 162.7 (J_{CF}=249 Hz), 147.1, 143.0, 136.8, 134.6 (J_{CF}=3.1 Hz), 134.0, 131.5, 131.4 (J_{CF}=8.5 Hz), 129.6, 125.5, 115.6 (J_{CF}=22.2 Hz), 92.4. ¹⁹F NMR (376 MHz, CDCl₃) δ −111.9 (1F, dddd, J_{FH}=8.5, 8.5, 5.3, 5.3 Hz). HRMS calc'd for C₁₄H₉NO₂FBr₂ ([MH]⁺) 399.8984. Found: 399.8984. Anal. Calc'd for C₁₄H₈NO₂FBr₂: C, 41.93; H, 2.01; N, 3.49. Found: C, 42.09; H, 2.01; N, 3.46.

### Step 3: Synthesis of 2,2-dibromo-1-(4-fluorophenyl)-1-(2-aminophenyl)ethene

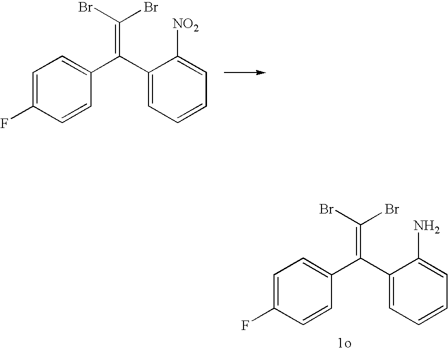

A mixture of 2,2-dibromo-1-(4-fluorophenyl)-1-(2-nitrophenyl)ethene (0.401 g, 1 mml) and iron powder (0.196 g, 3.5 mL) in acetic acid (2 mL) was heated to 115° C. (under reflux) for 2 h. The mixture was diluted with EtOAc (10 mL) and excess iron was removed by filtration through a celite pad. The mixture was washed with H₂O (2×10 mL), NaHCO₃ (Saturated, 10 mL), brine (5 mL) and dried over Na₂SO₄. The crude product after removal of solvent was purified by flash chromatography on silica gel (10% EtOAc in hexanes) to afford the product as a solid (0.260 g, 70%). R_{f}=0.22 (10% EtOAc in hexanes). mp 88-89° C. IR (neat, cm⁻¹) 3466 (m), 3380 (m), 1614 (s), 1502 (s), 1449 (m), 1301 (m), 1228 (s), 1158 (m). ¹H NMR (400 MHz, CDCl₃) δ 7.44-7.38 (2H, m), 7.14 (1H, ddd, J=7.7, 7.7, 1.5 Hz), 7.04-6.98 (3H, m), 6.77 (1H, ddd, J=7.5, 7.5, 1.1 Hz), 6.71 (1H, dd, J=8.1, 0.9 Hz), 3.75 (2H, s, br). ¹³C NMR (100 MHz, CDCl₃) δ 162.5 (J_{CF}=249 Hz), 144.5, 142.8, 135.7 (J_{CF}=3.8 Hz), 130.8 (J_{CF}=8.4 Hz), 129.7, 129.5, 127.9, 118.9, 116.5, 115.7 (J_{CF}=21.5 Hz), 92.4. ¹⁹F NMR (376 MHz, CDCl₃) δ −112.5 (1F, dddd, J_{FH}=8.5, 8.5, 5.3, 5.3 Hz). HRMS calc'd for C₁₄H₁₀NFBr₂ ([M]⁺) 368.9164. Found: 368.9175.

### Example 1p

### Alternate Reduction Conditions for Step 3, Example 1o (Synthesis of 2-[2,2-Dibromo-1-(4-fluoro-phenyl)-vinyl]-phenylamine)

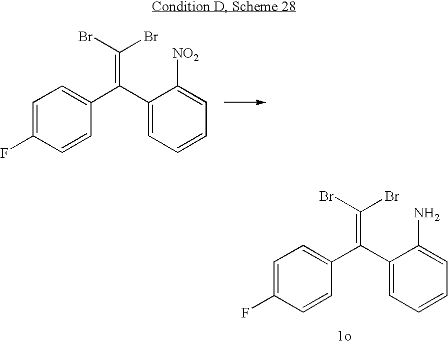 A mixture of 2,2-dibromo-1-(4-fluorophenyl)-1-(2-nitrophenyl)ethene (0.200 g, 0.5 mml) and platinum catalyst (20 mg) [1% on activated carbon, vanadium doped (50% wetted powder) Degussa F4 (Strem catalogue 2004-2006 78-1512)] in MeOH (2 mL) was hydrogenated under 1 atm H₂ for 6 hours until all the starting material was consumed. The catalyst was removed by filtration and the residue after removal of solvent was chromatographed with 10% EtOAc/hexanes to afford the product as a solid. (0.1735 g, 93%).The analytical data are identical to the product in example 1o. 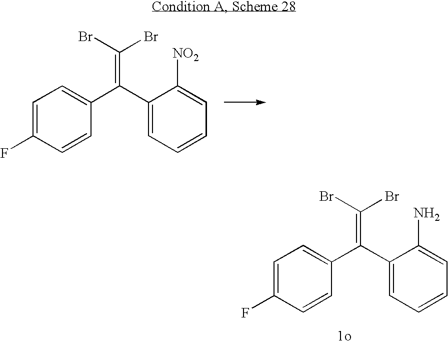
A mixture of 2,2-dibromo-1-(4-fluorophenyl)-1-(2-nitrophenyl)ethene (0.700 g, 1.75 mml) and SnCl₂.2H₂O (1.97 g) in EtOH (8 mL) were heated to 100° C. for 10 h. The mixture was cooled to rt and neutralized with K₂CO₃/H₂O. After extracted with EtOAc (4×30 mL), the organic was washed with brine and dried over Na₂SO₄. The residue after removal of solvent was chromatographed with 10% EtOAc/hexanes to afford a solid (0.258 g, 40%). The analytical data are identical to the product in example 1o. 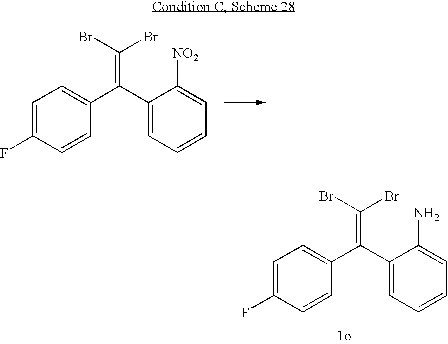
To a warm solution of 2,2-dibromo-1-(4-fluorophenyl)-1-(2-nitrophenyl)ethene (0.401 g, 1.0 mml) in HOAc (0.3 mL) and EtOH (2 mL) was added Fe powder (0.405 g, 7 mmol) and FeCl₃.6H₂O (30 mg). The mixture as stirred and heated to 100° C. for 2 h until the starting material was completely converted. The mixture as filtered through a celite pad and washed with copious amount of EtOAc. The solvent was removed and the residue was chromatographed with 10% EtOAc/Hexanes to afford the product as a solid (0.307 g, 83%). The analytical data are identical to the product in example 1o.

### Example 1q

### Synthesis of 2-(2,2-Dichloro-1-methyl-vinyl)-phenylamine

### Step 1: Synthesis of 1-(2,2-Dichloro-1-methyl-vinyl)-2-nitro-benzene

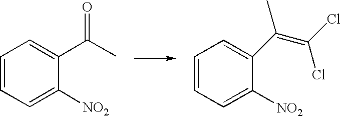

A modified literature procedure was applied to prepare 1-(2,2-dichloro-1-methyl-vinyl)-2-nitro-benzene (Olah, G. A.; Yamada, Y. J. Org. Chem. 1975, 40, 1107-1109). Potassium tert-butoxide was freshly prepared by dissolving metal potassium (4.0 g, 0.1 mol) in t-BuOH (˜100 mL) at rt. After most of the metal had disappeared (overnight), excess t-BuOH was removed by normal-pressure distillation. Residual t-BuOH was removed by azeotrope distillation with n-heptane (2×100 mL). The fresh t-BuOK was added n-heptane (350 mL), followed by PPh₃ (26.2 g, 0.1 mol) and the mixture was heated to 100° C. for 5 min and cooled to <5° C. with an ice bath. A chloroform (11.9 g, 0.1 mol) n-heptane (100 mL) solution was added dropwise to the mixture. After the addition, the mixture was stirred for another 30 min and warmed to rt. The mixture was concentrated to about 150 mL under rotary evaporator (high vacuum, rt water bath). To the mixture of the reagent was added a solution of 2′-nitroacetophenone (7.6 g, 0.046 mol) in benzene (100 mL) under 10° C. After addition, the mixture was slowly warmed to rt overnight and filtered through a celite pad. Solvent was removed and the residue was redissolved in Et₂O (100 mL). H₂O₂ (10%, 10 mL) was added to the mixture and stirred for half hour. Hexanes (200 mL) were added and triphenylphosphine oxide precipitate was removed by filtration. The organic phase was washed with H₂O (50 mL) and brine (20 mL) and dried over MgSO₄. The product was further purified by flash chromatography on silica gel (10% EtOAc in hexanes) to afford the desired product (10.0 g, 94%) as a light yellowish solid. ¹H NMR (300 MHz, CDCl₃) δ 8.10 (1H, dd, J=8.2, 1.2 Hz), 7.66 (1H, ddd, J=7.6, 7.6, 1.3 Hz), 7.51 (1H, ddd, J=8.1, 7.5, 1.5 Hz), 7.30 (1H, dd, J=7.7, 1.5 Hz), 2.22 (3H, s).

### Step 2: Synthesis of 2-(2,2-Dichloro-1-methyl-vinyl)-phenylamine

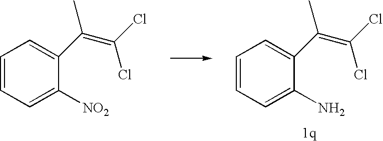

A mixture of the nitro compound (6.5 g, 28 mmol) and SnCl₂.2H₂O (31.6 g, 140 mmol) in EtOH (100 mL) was heated to 100° C. under reflux for 8 h. Most EtOH was removed under vacuum and the residue was diluted with EtOAc (50 mL). The mixture was neutralized by addition of K₂CO₃ and H₂O until PH>9. The heterogeneous mixture was extracted with EtOAc (4×30 mL), dried over Na₂SO₄. The product was further purified by flash chromatography on silica gel (10% EtOAc in hexanes) to afford an oil (5.3 g, 94%). ¹H NMR (300 MHz, CDCl₃) δ 7.13 (1H, ddd, J=7.9, 7.3, 1.6 Hz), 6.95 (1H, dd, J=7.6, 1.5 Hz), 6.77 (1H, ddd, J=7.5, 7.5, 1.1 Hz), 6.72 (1H, dd, J=8.1, 0.7 Hz), 3.65 (2H, br), 2.15 (3H, s).

### Example 1r

### 2-(2,2-Dibromo-vinyl)-6-methyl-phenylamine

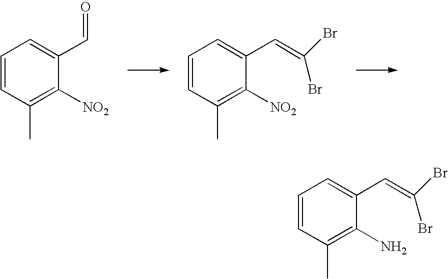

To a solution of 3-Methyl-2-nitro-benzaldehyde (1.40 g, 8.5 mmol) and CBr₄ (4.22 g, 12.7 mmol) in DCM (40 mL) at 0° C. was added dropwise a solution of PPh₃ (6.67 g, 25.44 mmol)) in DCM (40 mL) by an addition funnel. The addition rate was controlled so that the internal temperature was at 1-5° C. After addition, the mixture was stirred for another 1 h before warmed to rt and stirred for another 1 h. The reaction mixture was hexane (70 mL) and filtered through a short plug of silica gel and the silica gel was washed with copious amount of DCM until no product was found. The filtrate was collected and solvent was removed under vacuum. The residue was chromatographed with 5% EtOAc in hexane to afford the product 1-(2,2-Dibromo-vinyl)-3-methyl-2-nitro-benzene as a slightly yellow solid (2.30 g, 85%). R_{f}=0.35 (5% EtOAc in hexanes). IR (neat, cm⁻¹) 3028 (m), 1609 (m), 1527 (s), 1364 (s). ¹H NMR (400 MHz, CDCl₃) δ 7.48-7.40 (3H, m), 7.31 (1H, d, J=7.3 Hz), 2.38 (3H, s). ¹³C NMR (100 MHz, CDCl₃) δ 149.8, 131.9, 131.6, 130.8, 130.6, 128.9, 128.0, 95.6, 18.0. HRMS calc'd for C₉H₇NO₂Br₂ ([M]⁺) 318.8844. Found: 318.8850.

To a warm solution of 1-(2,2-Dibromo-vinyl)-3-methyl-2-nitro-benzene (0.321 g, 1 mmol) in HOAc (0.3 mL) and EtOH (2 mL) was added Fe powder (0.405 g, 7 mmol) and FeCl₃.6H₂O (36 mg). The mixture as stirred and heated to 100° C. for 2.5 h until the starting material was completely converted. The mixture as filtered through a celite pad and washed with copious amount of EtOAc. The solvent was removed and the residue was chromatographed with 10% EtOAc/Hexanes to afford the product as an oil (0.277 g, 95%). ¹H NMR (400 MHz, CDCl₃) δ 7.33 (1H, s), 7.15 (1H, d, J=7.7 Hz), 7.06 (1H, d, J=7.3 Hz), 6.71 (1H, t, J=7.6 Hz), 3.66 (2H, s, br), 2.10 (3H, s). ¹³C NMR (100 MHz, CDCl₃) δ 142.0, 134.7, 131.0, 127.3, 122.7, 121.6, 118.1, 93.1, 17.8. HRMS calc'd for C₉H₉NBr₂ ([M]⁺) 288.9102. Found: 288.9087.

### Example 1s

### 2-(2,2-Dibromo-vinyl)-naphthalen-1-ylamine

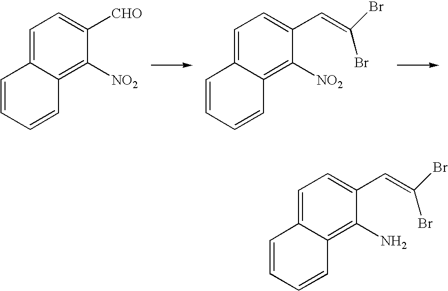

To a solution of 1-nitro-naphthalene-2-carbaldehyde (3.77 g, 18.7 mmol) and CBr₄ (9.31 g, 28.1 mmol) in DCM (100 mL) at 0° C. was added dropwise a solution of PPh₃ (14.7 g, 56.1 mmol)) in DCM (90 mL) by an addition funnel. The addition rate was controlled so that the internal temperature was at 1-5° C. After addition, the mixture was stirred for another 1 h before warmed to rt and stirred for another 0.5 h. The reaction mixture was hexane (70 mL) and filtered through a short plug of silica gel and the silica gel was washed with copious amount of 10% EtOAc/hexanes no product was found. The filtrate was collected and solvent was removed under vacuum. The residue was chromatographed with 10% EtOAc in hexane to afford the product 2-(2,2-dibromo-vinyl)-1-nitro-naphthalene as a off-white solid (5.50 g, 82%). IR (neat, cm⁻¹). ¹H NMR (400 MHz, CDCl₃) δ 8.00 (1H, d, J=8.8 Hz), 7.93-7.90 (1H, m), 7.85-7.82 (1H, m), 7.69 (1H, d, J=8.6 Hz), 7.68-7.63 (2H, m), 7.62 (1H, s). ¹³C NMR (100 MHz, CDCl₃) δ 146.6, 133.7, 131.6, 131.2, 129.3, 128.4, 128.3, 126.4, 125.7, 124.5, 122.3, 96.3. HRMS calc'd for C₁₂H₇NO₂Br₂ ([M]⁺) 354.8843. Found: 354.8840.

To a warm solution of 2-(2,2-dibromo-vinyl)-1-nitro-naphthalene (2.53 g, 7.09 mmol) in HOAc (2.5 mL) and EtOH (15 mL) was added Fe powder (2.84 g, 50 mmol) and FeCl₃.6H₂O (0.252 g). The mixture as stirred and heated to 100° C. for 1 h until the starting material was completely converted. The mixture as filtered through a celite pad and washed with copious amount of EtOAc. The solvent was removed and the residue was chromatographed with 7.5% EtOAc/Hexanes to afford the product as an yellow solid (2.035 g, 88%). ¹H NMR (400 MHz, CDCl₃) δ 7.82-7.76 (2H, m), 7.51 (1H, s), 7.50-7.43 (2H, m), 7.38 (1H, d, J=8.6 Hz), 7.28 (1H, d, J=8.6 Hz), 4.27 (2H, br). ¹³C NMR (100 MHz, CDCl₃) δ 139.4, 134.8, 134.3, 128.9, 126.7, 126.6, 125.6, 123.5, 121.0, 118.4, 116.1, 76.8. HRMS calc'd for C₁₂H₉Br₂ ([M]⁺) 324.9102. Found: 324.9089.

### Example 1t

### 2-(1-Dibromomethylene-3-phenyl-prop-2-ynyl)-phenylamine

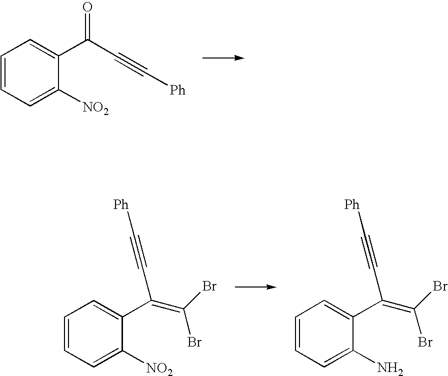

To a solution of 1-(2-nitro-phenyl)-3-phenyl-propynone (1.41 g, 5.6 mmol) and CBr₄ (2.78 g, 8.4 mmol) in DCM (50 mL) at 0° C. was added dropwise a solution of PPh₃ (4.41 g, 16.8 mmol)) in DCM (50 mL) by an addition funnel. The addition rate was controlled so that the internal temperature was at 1-5° C. After addition, the mixture was stirred for another 1 h. The reaction mixture was hexane (70 mL) and filtered through a short plug of silica gel and the silica gel was washed with copious amount of 10% EtOAc/hexanes no product was found. The filtrate was collected and solvent was removed under vacuum. The residue was chromatographed with 10% EtOAc in hexane to afford the product 1-(1-dibromomethylene-3-phenyl-prop-2-ynyl)-2-nitro-benzene as white solid (1.23 g, 54%). IR (neat, cm⁻¹). ¹H NMR (400 MHz, CDCl₃) ¹H NMR (400 MHz, CDCl₃) δ 8.12 (1H, dd, J=8.4, 1.1 Hz), 7.69 (1H, ddd, J=7.6, 7.6, 1.1 Hz), 7.57 (1H, ddd, J=8.3, 7.3, 1.5 Hz), 7.50 (1H, dd, J=7.7, 1.3 Hz), 7.45-7.43 (2H, m), 7.36-7.28 (3H, m). ¹³C NMR (100 MHz, CDCl₃) δ 147.1, 133.7, 133.4, 131.6, 131.2, 129.8, 129.2, 128.4, 127.6, 125.0, 122.1, 100.7, 98.0, 86.8. HRMS calc'd for C₁₆H₉NO₂Br₂ ([M]⁺) 404.9000. Found: 404.9002.

A mixture of 1-(1-dibromomethylene-3-phenyl-prop-2-ynyl)-2-nitro-benzene (1.018 g, 2.5 mml) and platinum catalyst (120 mg) [1% on activated carbon, vanadium doped (50% wetted powder) Degussa F4 (Strem catalogue 2004-2006 78-1512)] in MeOH (10 mL) was hydrogenated under 1 atm H₂ for 7 hours until all the starting material was consumed. The catalyst was removed by filtration and the solvent was removed under vacuum and the residue was chromatographed with 5% EtOAc/hexanes to afford the product as an oil (0.838 g, 89%). R_{f}=0.15 (5% EtOAc/hexanes). ¹H NMR (400 MHz, CDCl₃) δ 7.47-7.45 (2H, m), 7.36-7.26 (3H, m), 7.17 (1H, ddd, J=7.7, 7.7, 1.3 Hz), 7.13 (1H, dd, J=7.6, 1.2 Hz), 6.78 (1H, t, J=7.6 Hz), 6.72 (1H, d, J=8.1 Hz), 3.87 (2H, br). ¹³C NMR (100 MHz, CDCl₃) δ 143.4, 131.8, 130.1, 129.7, 129.2, 128.6, 123.7, 122.5, 118.6, 116.3, 102.1, 97.9, 87.6. ESI-HRMS calc'd for C₁₆H₁₂NBr₂ ([MH]⁺) 375.9330. Found: 375.9330.

### Example 1u

### Synthesis of 4-Amino-3-(2,2-dibromo-vinyl)-benzoic acid methyl ester

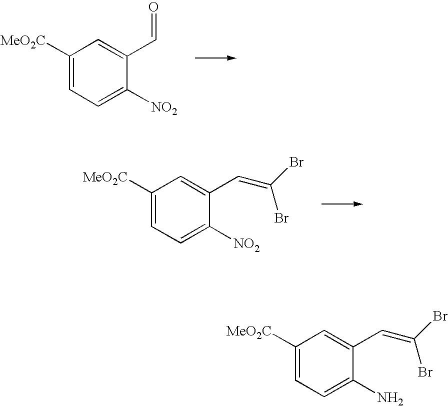

To a solution methyl 3-formyl-4-nitrobenzoate (3.137 g, 15 mmol) and CBr₄ (5.48 g, 16.5 mmol) in DCM (50 mL) was dropwise added PPh₃ (7.86 g, 30 mmol) solution in DCM (50 mL) at 0° C. After addition, the mixture was stirred for 1 h and warmed to rt. The solution was filtered through a short silica gel column, eluted with 20% EtOAc/hexanes. The solvent was evaporated and the residue was chromatographed with 10 to 20% EtOAc/hexanes to afford the product as a slightly yellow compound (5.23 g, 95.5%). ¹H NMR (400 MHz, CDCl₃) δ 8.27 (1H, t, J=0.8 Hz), 8.19-8.13 (2H, m), 7.76 (1H, s), 3.99 (3H, s). ¹³C NMR (100 MHz, CDCl₃) δ 164.9, 149.4, 134.7, 133.2, 133.1, 131.7, 130.6, 125.2, 94.9, 53.2. HRMS calc'd for C₁₀H₈NO₄Br₂ ([M]⁺) 363.8820. Found: 363.8823.

A mixture of 3-(2,2-dibromo-vinyl)-4-nitro-benzoic acid methyl ester (3.65 g, 10 mml) and platinum catalyst (365 mg) [1% on activated carbon, vanadium doped (50% wetted powder) Degussa F4 (Strem catalogue 2004-2006 78-1512)] in MeOH (30 mL) was hydrogenated under 1 atm H₂ for 8 hours until all the starting material was consumed. The catalyst was removed by filtration and the solvent was removed under vacuum to afford the product as analytically pure product without column chromatography. (3.35 g, 100%) The analytical data are identical to the sample prepared in example 1k.

### Example 1v

### {2-[2,2-Dibromo-1-(4-fluoro-phenyl)-vinyl]-phenyl}isopropylamine

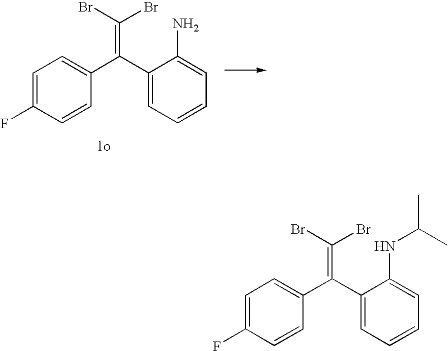

To a solution of 2-[2,2-dibromo-1-(4-fluoro-phenyl)-vinyl]phenylamine (1.855 g, 5 mmol) in 1,2-dichloroethane (15 mL) was added 2-methoxypropene (0.718 mL), HOAc (0.285 mL) and NaHB(OAc)₃ (1.59 g, 7.5 mmol). The mixture was stirred at rt for 17 h and quenched by addition of NaOH (1M, 20 mL), extracted with Et₂O (2×40 mL) and dried over Na₂SO₄. The residue after removal of solvent was chromatographed with 2.5% EtOAc/hexanes to afford the product as a solid after freezing (1.905 g, 92%). ¹H NMR (400 MHz, CDCl₃) δ 7.41-7.36 (2H, m), 7.19 (1H, ddd, J=8.1, 7.5, 1.5 Hz), 7.03 (1H, dd, J=7.5, 1.5 Hz), 7.01-6.96 (2H, m), 6.67 (1H, ddd, J=7.5, 7.5, 1.1 Hz), 6.65 (1H, d, J=8.3 Hz), 3.64-3.54 (2H, m), 1.20 (3H, d, J=3.2 Hz), 1.00 (3H, d, J=4.2 Hz). ¹³C NMR (100 MHz, CDCl₃) δ 162.5 (J_{CF}=248 Hz), 144.4, 143.8, 135.8 (J_{CF}=3.8 Hz), 130.9 (J_{CF}=7.7 Hz), 129.8, 129.6, 127.7, 116.7, 115.4 (J_{CF}=22.2 Hz), 112.2, 92.5 (J_{CF}=1.5 Hz), 44.3, 23.1. ESI-HRMS calc'd for C₁₇H₁₇NFBr₂ ([MH]⁺) 411.9706 Found: 411.9689.

### Example 1w

### 2-(2,2-Dichloro-vinyl)-phenylamine

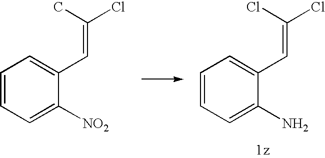

A mixture of 2-(2,2-dichlorovinyl)nitrobenzene (Olah, G. A.; Yamada, Y. J. Org. Chem. 1975, 40, 1107-1109) (0.100 g, 10 mml) and platinum catalyst (10 mg) [1% on activated carbon, vanadium doped (50% wetted powder) Degussa F4 (Strem catalogue 2004-2006 78-1512)] in MeOH (1 mL) was hydrogenated under 1 atm H₂ for 4 hours until all the starting material was consumed. The catalyst was removed by filtration and the solvent was removed under vacuum. The residue was chromatographed with 10% EtOAc/hexanes to afford the product as an off-white solid. (0.081 g, 94%). (Olah, G. A.; Yamada, Y. J. Org. Chem. 1975, 40, 1107-1109)

Preparation of 2-Substituted Indoles

The results of the preparation of various 2-substituted indoles of Tables 1 and 2 above are shown in Examples 2a-2cc below.

### Example 2a

### General Procedure A for Palladium-Catalyzed Tandem Reactions Using Boronic Acids—Preparation of 2-phenylindole

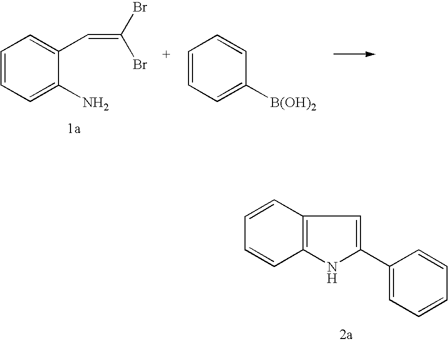

To a 10-mL round-bottom flask was charged with 2-(2,2-dibromo-vinyl)-phenylamine (0.277 g, 1 mmol), PhB(OH)₂ (0.183 g, 1.5 mmol) and powdered K₃PO₄-H₂O (1.15 g, 5 mmol) and the mixture was purged with Ar for at least 10 min. To a separate 10-mL round-bottom flask was charged with Pd(OAc)₂ (2.3 mg, 1 mol %) and s-Phos (8.2 mg, 2 mol %) and purged with Ar for at least 10 min. Dry toluene (5 mL) was added to the pre-catalyst flask and the mixture was stirred at rt for 3 min. The homogenous pre-catalyst solution was then cannulated to the reactant flask and the heterogenous mixture was stirred at rt for 2 min and heated to 90° C. After stirred at 90° C. for 6 h, the mixture was cooled to rt and diluted with Et₂O (15 mL). After aqueous workup, the mixture was purified by flash chromatography (10% EtOAc in hexanes) to afford a white crystalline solid (0.163 g, 84%). Its ¹H NMR spectrum was identical to an authentic sample.

### Example 2b

### Preparation of 2-(4-Methoxy-phenyl)-1H-indole

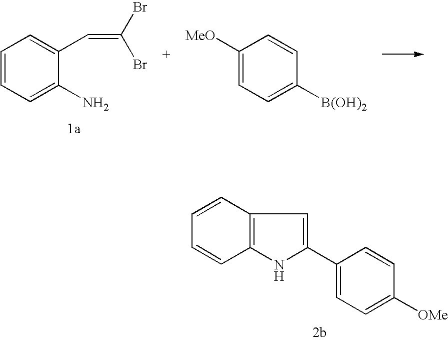

Following General Procedure A of Example 2a, a mixture of 2-(2,2-dibromo-vinyl)-phenylamine (0.277 g, 1 mmol), 4-methoxylphenylboronic acid (0.228 g, 1.5 mmol), K₃PO₄.H₂O (1.15 g, 5 mmol), and catalyst solution (Pd(OAc)₂ (2.3 mg, 1 mol %) and s-Phos (8.2 mg, 2 mol %) in PhMe (5 mL)) was heated at 90° C. for 2 h. After an aqueous workup, the crude was purified by flash chromatography on silica gel (10→20% EtOAc in hexanes) to afford a white crystalline solid (0.186 g, 83%) as the title product (Sezen, B.; Sames, D. J. Am. Chem. Soc. 2003, 125, 5274-5275).

### Example 2c

### Preparation of 2-o-Tolyl-1H-indole

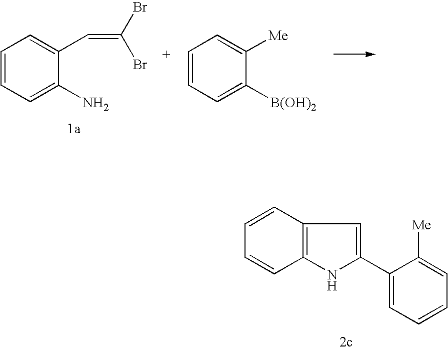

Following General Procedure A of Example 2a, a mixture of 2-(2,2-dibromo-vinyl)-phenylamine (0.277 g, 1 mmol), 2-methylphenylboronic acid (0.204 g, 1.5 mmol), K₃PO₄.H₂O (1.15 g, 5 mmol), and catalyst solution (Pd(OAc)₂ (2.3 mg, 1 mol %) and s-Phos (8.2 mg, 2 mol %) in PhMe (5 mL)) was heated at 90° C. for 4 h. After an aqueous workup, the crude was purified by flash chromatography on silica gel (5% EtOAc in hexanes) to afford a white crystalline solid (0.170 g, 82%).

### Example 2d

### Preparation of 2-p-Tolyl-1H-indole

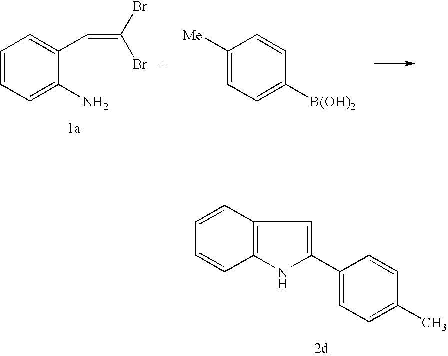

Following General Procedure A of Example 2a, a mixture of 2-(2,2-dibromo-vinyl)-phenylamine (0.139 g, 0.5 mmol), 4-methylphenylboronic acid (0.102 g, 0.75 mmol), K₃PO₄.H₂O (0.58 g, 2.5 mmol), and catalyst solution (Pd(OAc)₂ (1.2 mg, 1 mol %) and s-Phos (4.1 mg, 2 mol %) in PhMe (2.5 mL)) was heated at 90° C. for 5 h. After an aqueous workup, the crude was purified by flash chromatography on silica gel (10% EtOAc in hexanes) to afford a white crystalline solid (0.091 g, 88%).

### Example 2e

### Preparation of 2-(4-Methoxy-2-methyl-phenyl)-1H-indole

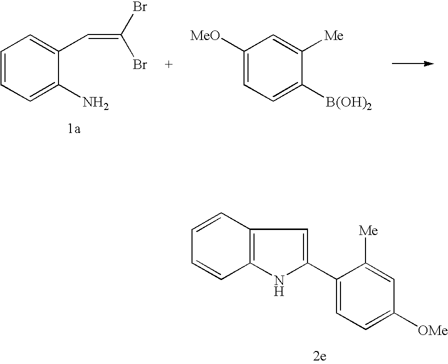

Following General procedure A of Example 2a, a mixture of 2-(2,2-dibromo-vinyl)-phenylamine (0.277 g, 1 mmol), 4-methoxyl-2-methylphenylboronic acid (0.249 g, 1.5 mmol), K₃PO₄.H₂O (1.15 g, 5 mmol), and catalyst solution (Pd(OAc)₂ (2.3 mg, 1 mol %) and s-Phos (8.2 mg, 2 mol %) in PhMe (5 mL)) was heated at 90° C. for 5.5 h. After an aqueous workup, the crude was purified by flash chromatography on silica gel (5→10% EtOAc in hexanes) to afford a white crystalline solid (0.187 g, 79%) as the title product (Pigerol, C.; Chandavoine, M. M.; De Cointet de Fillain, P.; Nanthavong, S. In Ger. Offen.; (Labaz, Fr.). De, 1975, p 37 pp). This indole is known as an anti-oxidant for use in food preservation.

### Example 2f

### Preparation of 2-(4-Trifluoromethyl-phenyl)-1H-indole

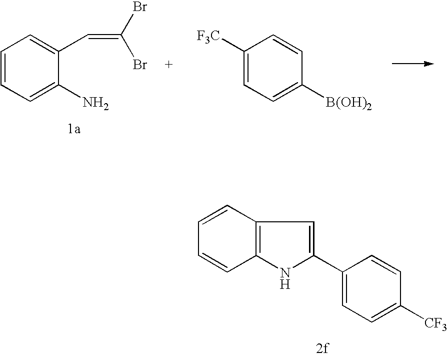

Following General Procedure A of Example 2a, a mixture of 2-(2,2-dibromo-vinyl)-phenylamine (0.277 g, 1 mmol), 4-trifluoromethylphenylboronic acid (0.285 g, 1.5 mmol), K₃PO₄.H₂O (1.15 g, 5 mmol), and catalyst solution (Pd(OAc)₂ (2.3 mg, 1 mol %) and s-Phos (8.2 mg, 2 mol %) in PhMe (5 mL)) was heated at 90° C. for 7 h. After an aqueous workup, the crude was purified by flash chromatography on silica gel (5% EtOAc in hexanes) to afford a white crystalline solid (0.196 g, 75%).

### Example 2g

### 2-Naphthalen-2-yl-1H-indole

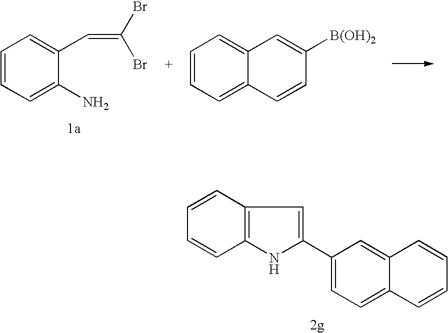

Following General Procedure A of Example 2a, a mixture of 2-(2,2-dibromo-vinyl)-phenylamine (0.277 g, 1 mmol), 2-naphthaleneboronic acid (0.258 g, 1.5 mmol), K₃PO₄.H₂O (1.15 g, 5 mmol), and catalyst solution (Pd(OAc)₂ (2.3 mg, 1 mol %) and s-Phos (8.2 mg, 2 mol %) in PhMe (5 mL)) was heated at 90° C. for 7 h. After an aqueous workup, the crude was purified by flash chromatography on silica gel (10% EtOAc in hexanes) to afford a white crystalline solid (0.199 g, 82%) as the title product (Baumgartner, M. T.; Nazareno, M. A.; Murguia, M. C.; Pierini, A. B.; Rossi, R. A. Synthesis 1999, 2053-2056).

### Example 2h

### Preparation of 2-(3-chloro-phenyl)-1H-indole

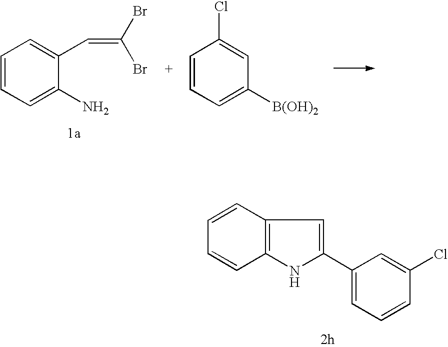

Following general procedure A of Example 2a, a mixture of 2-(2,2-dibromo-vinyl)-phenylamine (0.083 g, 0.3 mmol), 3-chlorophenylboronic acid (0.070 g, 0.45 mmol), K₃PO₄.H₂O (0.35 g, 1.5 mmol), and catalyst solution (Pd(OAc)₂ (2.3 mg, 3.3 mol %) and s-Phos (8.2 mg, 6.6 mol %) in PhMe (1.5 mL)) was heated at 90° C. for 2.5 h. After an aqueous workup, the crude was purified by flash chromatography on silica gel (10% EtOAc in hexanes) to afford a white crystalline solid (0.041 g, 60%) (Inion, H.; De Vogelaer, H.; Van Durme, E.; Descamps, M.; Brotelle, R.; Charlier, R.; Colot, M. Eur. J. Med. Chem. 1975, 10, 276-285).

### Example 2i

### Preparation of 2-(4-chloro-phenyl)-1H-indole

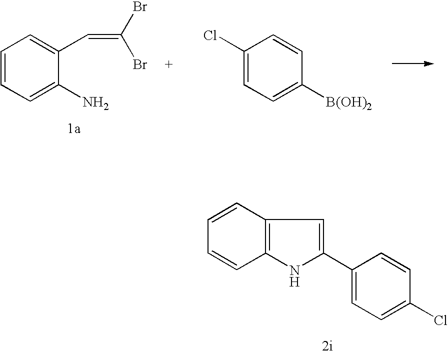

Following general procedure A of Example 2A, a mixture of 2-(2,2-dibromo-vinyl)-phenylamine (0.083 g, 0.3 mmol), 4-chlorophenylboronic acid (0.070 g, 0.45 mmol), K₃PO₄.H₂O (0.35 g, 1.5 mmol), and catalyst solution (Pd(OAc)₂ (2.3 mg, 3.3 mol %) and s-Phos (8.2 mg, 6.6 mol %) in PhMe (1.5 mL)) was heated at 90° C. for 2.5 h. After an aqueous workup, the crude was purified by flash chromatography on silica gel (10% EtOAc in hexanes) to afford a white crystalline solid (0.037 g, 57%).

### Example 2j

### Preparation of 2-Thiophen-3-yl-1H-indole

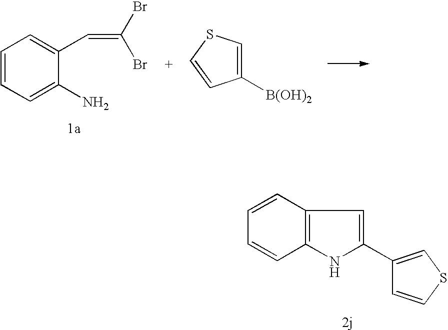

Following general procedure A of Example 2a, a mixture of 2-(2,2-dibromo-vinyl)-phenylamine (0.139 g, 0.5 mmol), 3-thiopheneboronic acid (0.096 g, 0.75 mmol), K₃PO₄.H₂O (0.58 g, 2.5 mmol), and catalyst solution (Pd(OAc)₂ (2.3 mg, 2 mol %) and s-Phos (8.2 mg, 4 mol %) in PhMe (2.5 mL)) was heated at 90° C. for 12 h. After an aqueous workup, the crude was purified by flash chromatography on silica gel (7% EtOAc in hexanes) to afford a white crystalline solid (0.086 g, 86%). R_{f}=0.20 (7% EtOAc/Hexanes). mp 212-214° C. (sealed). IR (neat, cm⁻¹) 3416 (m), 3091(m), 1452 (w), 1418 (s), 1340 (m), 1085 (m). ¹H NMR (400 MHz, CDCl₃) δ 8.17 (1H, br), 7.60 (1H, d, J=7.7 Hz), 7.39 (2H, s), 7.36 (1H, d, J=7.7 Hz), 7.24 (1H, s), 7.18 (1H, ddd, J=7.6. 7.6, 1.1 Hz), 7.11 (1H, ddd, J=7.4. 7.4, 1.1 Hz), 6.70 (1H, d, J=1.3 Hz). ¹³C NMR (100 MHz, CDCl₃) δ 136.6, 134.3, 134.1, 129.3, 126.9, 125.9, 122.5, 120.8, 120.5, 119.3, 110.9, 100.2. HRMS calc'd for C₁₂H₉NS (M⁺) 199.0456. Found: 199.0453.

### Example 2k

### Preparation of 2-Hex-1-enyl-1H-indole

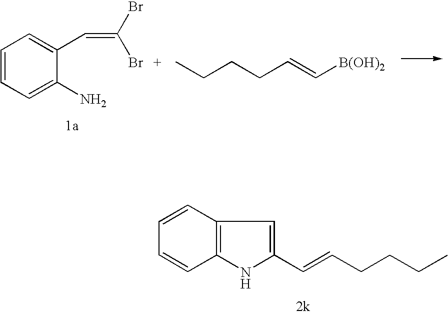

Following general procedure A of Example 2a, a mixture of 2-(2,2-dibromo-vinyl)-phenylamine (0.139 g, 0.5 mmol), trans-1-hexenylboronic acid (0.128 g, 1 mmol), K₃PO₄.H₂O (0.58 g, 2.5 mmol), and catalyst solution (Pd(OAc)₂ (2.3 mg, 2 mol %) and s-Phos (8.2 mg, 4 mol %) in PhMe (2.5 mL)) was heated at 90° C. for 5 h. After an aqueous workup, the crude was purified by flash chromatography on silica gel (5% EtOAc in hexanes) to afford a white crystalline solid (0.080 g, 80%). R_{f}=0.23 (5% EtOAc/Hexanes). mp 70-72° C. (hexanes). IR (neat, cm⁻¹) 3420 (m), 3382 (m), 2925 (m), 2867 (m), 1453 (m), 1413 (s), 1342 (w), 1293 (w), 1233 (w). ¹H NMR (400 MHz, CDCl₃) δ 8.02 (1H, br), 7.53 (1H, d, J=7.9 Hz), 7.27 (1H, d, J=8.1 Hz), 7.13 (1H, ddd, J=7.6. 7.6, 1.1 Hz), 7.06 (1H, ddd, J=7.4. 7.4, 0.9 Hz), 6.39 (1H, d, J=14.3 Hz), 6.38 (1H, s), 6.03 (1H, ddd, J=16.1, 7.0, 7.0 Hz), 2.23 (2H, dddd, J=7.2, 7.2, 7.2, 1.1 Hz), 1.50-1.33 (4H, m), 0.93 (3H, t, J=7.1 Hz). ¹³C NMR (100 MHz, CDCl₃) δ 136.8, 136.6, 130.5, 129.2, 122.3, 120.9, 120.5, 120.1, 110.6, 101.5, 32.9, 31.6, 22.5, 14.2. HRMS calc'd for C₁₄H₁₇N (M⁺) 199.1361. Found: 199.1365.

### Example 2l

### Preparation of 2-Styryl-1H-indole

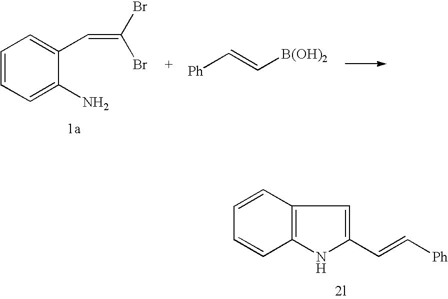

Following general procedure A of Example 2a, a mixture of 2-(2,2-dibromo-vinyl)-phenylamine (0.139 g, 0.5 mmol), trans-1-hexenylboronic acid (0.111 g, 0.75 mmol), K₃PO₄.H₂O (0.58 g, 2.5 mmol), and catalyst solution (Pd(OAc)₂ (2.3 mg, 2 mol %) and s-Phos (8.2 mg, 4 mol %) in PhMe (2.5 mL)) was heated at 90° C. for 7 h. After an aqueous workup, the crude was purified by flash chromatography on silica gel (5→10% EtOAc in hexanes) to afford a white crystalline solid (0.075 g, 68%).

### Example 2m

### Preparation of 2-(1-Ethyl-but-1-enyl)-1H-indole

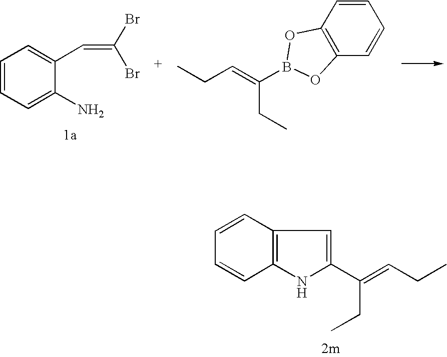

Following general procedure A of Example 2a, a mixture of 2-(2,2-dibromo-vinyl)-phenylamine (0.134 g, 0.48 mmol), 2-(cis-1-thyl-but-1-enyl)-benzo[1,3,2]dioxaborole (0.125 g, 1.2 mmol), K₃PO₄.H₂O (0.58 g, 2.5 mmol), and catalyst solution (Pd(OAc)₂ (3.5 mg, 3 mol %) and s-Phos (12.3 mg, 6 mol %) in PhMe (2.5 mL)) was heated at 90° C. for 6 h. After an aqueous workup, the crude was purified by flash chromatography on silica gel (5% EtOAc in hexanes) to afford a white crystalline solid (0.070 g, 73%) as the title product (Ayguen, A.; Pindur, U. J. Heterocycl. Chem. 2003, 40, 411-417).

### Example 2n

### General Procedure B for Palladium-Catalyzed Tandem Reactions Using a Trialkylborane—Preparation of 2-Ethyl-1H-indole

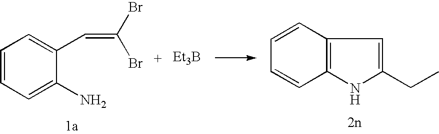

To a round-bottom flask was charged with 2-(2,2-dibromo-vinyl)-phenylamine (0.139 g, 0.50 mmol), K₃PO₄.H₂O (0.58 g, 2.5 mmol), Pd₂(dba)₃ (4.6 mg, 2 mol % Pd) and s-Phos (10.3 mg, 5 mol %). After the mixture was purged with N₂ for over 10 min, triethylborane was added, followed by addition of H₂O (10 μL). The reaction mixture was stirred at 60° C. for 2 h. The mixture was then cooled to −20° C., to which H₂O₂ (30%, 0.5 mL) was added. The mixture was slowly warmed to rt and stirred for another 30 min. After usual aqueous workup, the product was purified by flash chromatography on silica gel (10% EtOAc in hexanes) to afford a crystalline product (0.108 g, 77%) as the title product (Sadanandan, E. V.; Srinivasan, P. C. Synthesis 1992, 648-650).

### Example 2o

### General Procedure C for Palladium-Catalyzed Tandem Reactions Using Alkyl 9-BBN—Preparation of 2-(4-Benzyloxy-butyl)-1H-indole

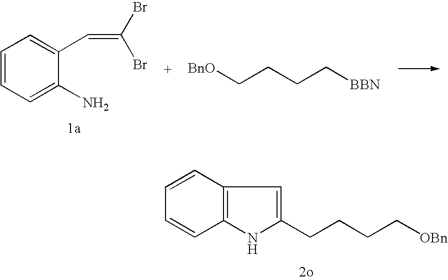

To a flame-dried round-bottom flask under N₂ was added 9-BBN solution (0.5 M, 1.65 mL, 0.825 mmol), followed by dropwise addition of but-3-enyloxymethyl-benzene (0.122 g, 0.75 mmol). The mixture was stirred at rt overnight (12 h). In a separate round-bottom flask was charged with 2-(2,2-dibromo-vinyl)-phenylamine (0.139 g, 0.50 mmol), K₃PO₄.H₂O (0.58 g, 2.5 mmol), Pd₂(dba)₃ (4.6 mg, 2 mol % Pd) and s-Phos (10.3 mg, 5 mol %). After the mixture was purged with N₂ for over 10 min, the alkyl 9-BBN solution was cannulated into the flask, followed by addition of H₂O (10 μL). The reaction mixture was stirred at 60° C. for 4 h. The mixture was then cooled to −20° C., to which H₂O₂ (30%, 0.5 mL) was added. The mixture was slowly warmed to rt and stirred for another 30 min. After usual aqueous workup, the product was purified by flash chromatography on silica gel (10% EtOAc in hexanes) to afford a white crystalline product (0.108 g, 77%). R_{f}=0.20 (15% EtOAc/Hexanes). mp 48-50° C. IR (neat, cm⁻¹) 3394 (s), 2935 (m), 2864 (m), 1550 (w), 1494 (m), 1455 (s), 1412 (m), 1367 (m), 1284 (m), 1122 (s). ¹H NMR (300 MHz, CDCl₃) δ 8.03 (1H, br), 7.51 (1H, d, J=7.7 Hz), 7.34-7.25 (5H, m), 7.23 (1H, d, J=7.4 Hz), 7.12-7.02 (2H, m), 6.22 (1H, s), 4.51 (2H, s), 3.53 (2H, t, J=5.9 Hz), 2.77 (2H, t, J=7.1 Hz), 1.87-1.80 (2H, m), 1.79-1.71 (2H, m). ¹³C NMR (75 MHz, CDCl₃) δ 139.8, 138.6, 136.0, 129.0, 128.6, 127.9, 127.9, 121.1, 119.9, 119.7, 110.5, 99.7, 73.3, 70.4, 29.3, 28.0, 26.5. Anal. Calc'd for C₁₉H₂₁NO: C, 81.68; H, 7.58; N, 5.01. Found: C, 81.60; H, 7.74; N, 5.11.

### Example 2p

### Preparation of 2-Hexyl-1H-indole

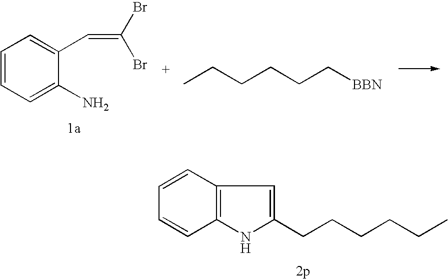

Following General Procedure C of Example 2o, n-hexyl 9-BBN was prepared from 1-hexene (0.063 g, 0.75 mmol) and 9-BBN (0.5M, 1.65 mL, 0.825 mmol). Reaction of n-hexyl 9-BBN, 2-(2,2-dibromo-vinyl)-phenylamine (0.139 g, 0.50 mmol), K₃PO₄.H₂O (0.58 g, 2.5 mmol), Pd₂(dba)₃ (4.6 mg, 2 mol % Pd), s-Phos (10.3 mg, 5 mol %) and H₂O (10 μL) at 60° C. for 3 h to afford the product as an oil (0.080 g, 79%) after purification by flash chromatography on silica gel (5% EtOAc in hexanes) as the title product (Ishikura, M.; Agata, I. Heterocycles 1995, 41, 2437-2440).

### Example 2q

### Preparation of 1-Benzyl-2-phenyl-1H-indole

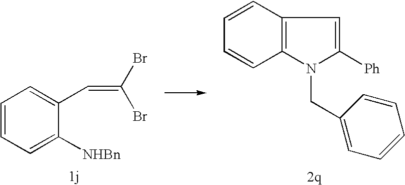

Following General Procedure A of Example 2a, a mixture of Benzyl-[2-(2,2-dibromo-vinyl)-phenyl]-amine (0.184 g, 0.50 mmol), PhB(OH)₂ (0.092 g, 0.75 mmol), K₃PO₄.H₂O (0.58 g, 2.5 mmol), and catalyst solution (Pd(OAc)₂ (1.2 mg, 1 mol %) and s-Phos (4.1 mg, 2 mol %) in PhMe (2.5 mL)) was heated at 90° C. for 4 h. After an aqueous workup, the crude was purified by flash chromatography on silica gel (2.5% EtOAc in hexanes) to afford a white crystalline solid (0.116 g, 82%) as the title product (Watanabe, T.; Kobayashi, A.; Nishiura, M.; Takahashi, H.; Usui, T.; Kamiyama, I.; Mochizuki, N.; Noritake, K.; Yokoyama, Y.; Murakami, Y. Chem. Pharm. Bull. 1991, 39, 1152-1156).

### Example 2r

### Preparation of 4-Methyl-2-phenyl-1H-indole

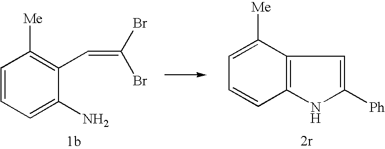

Following General Procedure A of Example 2a, a mixture of 2-(2,2-dibromo-vinyl)-3-methyl-phenylamine (0.147 g, 0.53 mmol), PhB(OH)₂ (0.092 g, 0.75 mmol), K₃PO₄.H₂O (0.58 g, 2.5 mmol), and catalyst solution (Pd(OAc)₂ (5.6 mg, 5 mol %) and s-Phos (20.5 mg, 10 mol %) in PhMe (2.5 mL)) was heated at 90° C. for 2 h. After an aqueous workup, the crude was purified by flash chromatography on silica gel (5% EtOAc in hexanes) to afford a white crystalline solid (0.080 g, 77%) as the title product (Rutherford, J. L.; Rainka, M. P.; Buchwald, S. L. J. Am. Chem. Soc. 2002, 124, 15168-15169).

### Example 2s

### Preparation of 4-Fluoro-2-phenyl-1H-indole

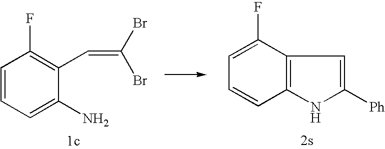

Following General Procedure A of Example 2a, a mixture of 2-(2,2-dibromo-vinyl)-3-fluoro-phenylamine (0.152 g, 0.515 mmol), PhB(OH)₂ (0.092 g, 0.75 mmol), K₃PO₄.H₂O (0.58 g, 2.5 mmol), and catalyst solution (Pd(OAc)₂ (1.2 mg, 1 mol %) and s-Phos (4.2 mg, 2 mol %) in PhMe (2.5 mL)) was heated at 90° C. for 14 h. After an aqueous workup, the crude was purified by flash chromatography on silica gel (7.5% EtOAc in hexanes) to afford a white crystalline solid (0.096 g, 88%). R_{f}=0.14 (7.5% EtOAc/Hexanes). mp 65-67° C. IR (neat, cm⁻¹) 3453 (s), 1583 (m), 1487 (m), 1453 (m), 1404 (m), 1358 (s), 1340 (s), 1226 (m), 1066 (m). ¹H NMR (300 MHz, CDCl₃) δ 8.37 (1H, br), 7.65-7.61 (2H, m), 7.46-7.40 (2H, m), 7.33 (1H, dddd, J=7.3, 7.3, 1.2, 1.2 Hz), 7.16 (1H, dd, J=8.2, 0.9 Hz), 7.09 (1H, ddd, J=7.9, 7.9, 4.9 Hz), 6.88 (1H, dd, J=2.5, 0.8 Hz), 6.79 (1H, ddd, J=10.3, 7.7, 1.0 Hz). ¹³C NMR (75 MHz, CDCl₃) δ 156.5 (J_{CF}=247 Hz), 139.4 (J_{CF}=11.2 Hz), 138.1, 132.0, 129.3, 128.3, 125.4, 122.9 (J_{CF}=7.4 Hz), 118.6 (J_{CF}=22.3 Hz), 107.2 (J_{CF}=3.7 Hz), 105.2 (J_{CF}=18.9 Hz), 96.0 (J_{CF}=0.6 Hz). ¹⁹F NMR (282 MHz, CDCl₃) δ −122.1 (1F, dd, J_{FH}=8.0, 5.7, 3.5 Hz). Anal. Calc'd for C₁₄H₁₀NF: C, 79.60; H, 4.77; N, 6.63. Found: C, 79.37; H, 5.13; N, 6.63.

### Example 2t

### Preparation of 5-Fluoro-2-phenyl-1H-indole

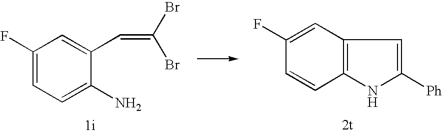

Following General Procedure A of Example 2a, a mixture of 2-(2,2-dibromo-vinyl)-4-fluoro-phenylamine (0.150 g, 0.51 mmol), PhB(OH)₂ (0.092 g, 0.75 mmol), K₃PO₄.H₂O (0.58 g, 2.5 mmol), and catalyst solution (Pd(OAc)₂ (1.2 mg, 1 mol %) and s-Phos (4.2 mg, 2 mol %) in PhMe (2.5 mL)) was heated at 90° C. for 2 h. After an aqueous workup, the crude was purified by flash chromatography on silica gel (10% EtOAc in hexanes) to afford a white crystalline solid (0.094 g, 87%) as the title product (Rowley, M.; Hallett, D. J.; Goodacre, S.; Moyes, C.; Crawforth, J.; Sparey, T. J.; Patel, S.; Marwood, R.; Patel, S.; Thomas, S.; Hitzel, L.; O'Connor, D.; Szeto, N.; Castro, J. L.; Hutson, P. H.; MacLeod, A. M. J. Med. Chem. 2001, 44, 1603-1614).

### Example 2u

### Preparation of 6-Fluoro-2-phenyl-1H-indole

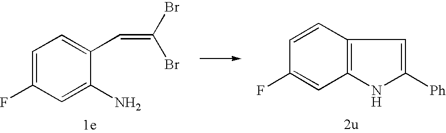

Following General Procedure A of Example 2a, a mixture of 2-(2,2-dibromo-vinyl)-5-fluoro-phenylamine (0.148 g, 0.50 mmol), PhB(OH)₂ (0.092 g, 0.75 mmol), K₃PO₄.H₂O (0.58 g, 2.5 mmol), and catalyst solution (Pd(OAc)₂ (2.3 mg, 2 mol %) and s-Phos (8.2 mg, 4 mol %) in PhMe (2.5 mL)) was heated at 90° C. for 2.5 h. After an aqueous workup, the crude was purified by flash chromatography on silica gel (5% EtOAc in hexanes) to afford a white crystalline solid (0.085 g, 80%).

### Example 2v

### Preparation of 2-Phenyl-6-trifluoromethyl-1H-indole

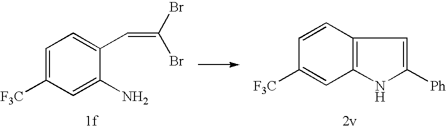

Following General Procedure A of Example 2a, a mixture of 2-(2,2-dibromo-vinyl)-5-trifluoromethyl-phenylamine (0.172 g, 0.50 mmol), PhB(OH)₂ (0.092 g, 0.75 mmol), K₃PO₄.H₂O (0.58 g, 2.5 mmol), and catalyst solution (Pd(OAc)₂ (1.2 mg, 1 mol %) and s-Phos (4.1 mg, 2 mol %) in PhMe (2.5 mL)) was heated at 90° C. for 2.5 h. After an aqueous workup, the crude was purified by flash chromatography on silica gel (5% EtOAc in hexanes) to afford a white crystalline solid (0.118 g, 90%).

### Example 2w

### Preparation of 2-Phenyl-1H-indole-6-carboxylic acid methyl ester

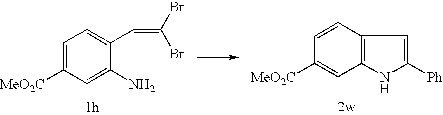

Following General Procedure A of Example 2a, a mixture of 3-amino-4-(2,2-dibromo-vinyl)-benzoic acid methyl ester (0.168 g, 0.50 mmol), PhB(OH)₂ (0.092 g, 0.75 mmol), K₃PO₄.H₂O (0.58 g, 2.5 mmol), and catalyst solution (Pd(OAc)₂ (1.2 mg, 1 mol %) and s-Phos (4.1 mg, 2 mol %) in PhMe (2.5 mL)) was heated at 90° C. for 8.5 h. After an aqueous workup, the crude was purified by flash chromatography on silica gel (10 →20% EtOAc in hexanes) to afford a white crystalline solid (0.113 g, 90%). R_{f}=0.23 (20% EtOAc/Hexanes). mp 208-210° C. IR (neat, cm⁻¹) 3347 (m), 1694 (s), 1620 (m), 1504 (m), 1435 (m), 1317 (m), 1284 (s), 1232 (s). ¹H NMR (300 MHz, DMSO-d₆) δ 11.95 (1H, s), 8.06 (1H, s), 7.91 (2H, d, J=8.2 Hz), 7.63 (2H, s), 7.51 (2H, t, J=7.6 Hz), 7.38 (1H, t, J=7.0 Hz), 7.02 (1H, s), 3.86 (3H, s). ¹³C NMR (75 MHz, DMSO-d₆) δ 167.2, 141.4, 136.3, 132.4, 131.5, 129.1, 128.3, 125.5, 122.4, 120.2, 119.8, 113.1, 99.2, 51.9. Anal. Calc'd for C₁₆H₁₃NO₂: C, 76.48; H, 5.21; N, 5.57. Found: C, 76.49; H, 5.41; N, 5.62. HRMS calc'd for C₁₆H₁₃NO₂ (MH⁺) 251.0946. Found: 251.0943.

### Example 2x

### Preparation of 5,6-Bis-benzyloxy-2-phenyl-1H-indole

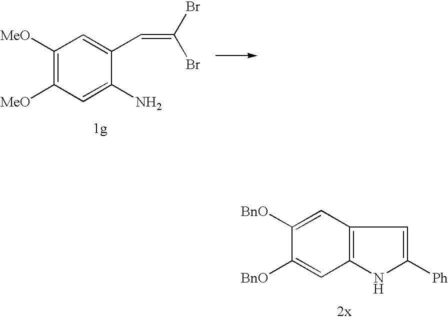

Following General Procedure A of Example 2a, a mixture of 2-(2,2-dibromo-vinyl)-4,5-dimethoxy-phenylamine (0.147 g, 0.30 mmol), PhB(OH)₂ (0.055 g, 0.45 mmol), K₃PO₄.H₂O (0.35 g, 1.5 mmol), and catalyst solution (Pd(OAc)₂ (2.3 mg, 3.3 mol %) and s-Phos (8.2 mg, 6.6 mol %) in PhMe (1.5 mL)) was heated at 90° C. for 4.5 h. After an aqueous workup, the crude was purified by flash chromatography on silica gel (10→15→20% EtOAc in hexanes) to afford a white crystalline solid (0.069 g, 57%). R_{f}=0.20 (20% EtOAc/Hexanes). mp 140-141° C. IR (neat, cm⁻¹) 3396 (m), 1602 (m), 1450 (s), 1337 (m), 1300 (m), 1243 (m), 1206 (s), 1132 (s). ¹H NMR (300 MHz, CDCl₃) δ 8.18 (1H, br), 7.54 (2H, d, J=7.4 Hz), 7.50-7.24 (13H, m), 7.16 (1H, s), 6.90 (1H, s), 6.66 (1H, s), 5.17 (2H, s), 5.14 (2H, s). ¹³C NMR (75 MHz, CDCl₃) δ 147.3, 145.3, 138.0, 137.7, 137.3, 132.7, 132.1, 129.1, 128.7, 128.6, 127.9, 127.9, 127.7, 127.6, 127.4, 124.9, 123.2, 106.8, 99.9, 98.3, 72.5, 72.1. Anal. Calc'd for C₂₈H₂₃NO₂: C, 82.94; H, 5.72; N, 3.45. Found: C, 82.62; H, 6.05; N, 3.49.

### Example 2y

### Preparation of 5-Benzyloxy-2-phenyl-1H-indole

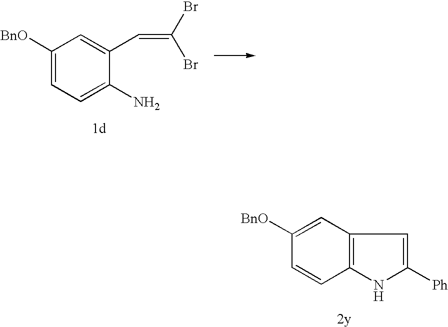

Following General Procedure A of Example 2a, a mixture of 4-benzyloxy-2-(2,2-dibromo-vinyl)-phenylamine (0.193 g, 0.50 mmol), PhB(OH)₂ (0.092 g, 0.75 mmol), K₃PO₄.H₂O (0.58 g, 2.5 mmol), and catalyst solution (Pd(OAc)₂ (2.3 mg, 2 mol %) and s-Phos (8.2 mg, 4 mol %) in PhMe (2.5 mL)) was heated at 90° C. for 3 h. After an aqueous workup, the crude was purified by flash chromatography on silica gel (15% EtOAc in hexanes) to afford a white crystalline solid (0.130 g, 86%). R_{f}=0.27 (15% EtOAc/Hexanes). mp 168-170° C. IR (neat, cm⁻¹) 3441 (s), 1586 (m), 1449 (s), 1409 (m), 1214 (m), 1154 (m). ¹H NMR (400 MHz, CDCl₃) δ 8.20 (1H, br), 7.62 (2H, d, J=7.8 Hz), 7.48 (2H, d, J=7.1 Hz), 7.41 (2H, t, J=7.7 Hz), 7.38 (2H, t, J=7.2 Hz), 7.31 (1H, t, J=7.8 Hz), 7.30 (1H, t, J=8.0 Hz), 7.27 (1H, d, J=7.8 Hz), 7.16 (1H, d, J=2.2 Hz), 6.93 (1H, dd, J=8.8, 2.4 Hz), 6.73 (1H, d, J=2.0 Hz), 5.11 (2H, s). ¹³C NMR (100 MHz, CDCl₃) δ 153.9, 138.8, 137.9, 132.6, 132.4, 129.9, 129.2, 128.7, 128.0, 127.9, 127.8, 125.3, 113.5, 111.8, 104.1, 100.1, 71.0. HRMS (ESI) calc'd for C₂₁H₁₈NO (MH⁺) 300.1382. Found: 300.1395.

### Example 2z

### Preparation of 2-phenylindole

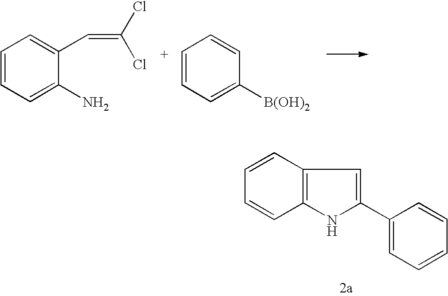

Following General Procedure A of Example 2a, a mixture of 2-(2,2-dichloro-vinyl)-phenylamine (0.094 g, 0.50 mmol), PhB(OH)₂ (0.092 g, 0.75 mmol), K₃PO₄.H₂O (0.58 g, 2.5 mmol), and catalyst solution (Pd(OAc)₂ (5.6 mg, 5 mol %) and s-Phos (20.5 mg, 10 mol %) in PhMe (2.5 mL)) was heated at 90° C. for 2 h. After an aqueous workup, the crude was purified by flash chromatography on silica gel (10% EtOAc in hexanes) to afford a white crystalline solid (0.093 g, 95%).

### Example 2aa

### Preparation of 2-Phenyl-1H-indole-5-carboxylic acid methyl ester

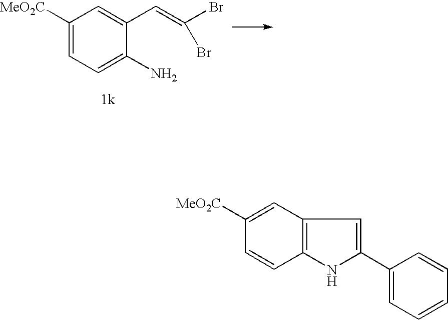

Following General Procedure A of Example 2a, a mixture of 4-amino-3-(2,2-dibromo-vinyl)-benzoic acid methyl ester (0.100 g, 0.30 mmol), PhB(OH)₂ (0.055 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 1.5 h. After an aqueous workup, the crude was purified by flash chromatography on silica gel (20% EtOAc in hexanes) to afford a white crystalline solid (0.066 g, 87%). This compound was previously prepared in the prior art (Fagnola, M. C.; Candiani, I.; Visentin, G.; Cabri, W.; Zarini, F.; Mongelli, N.; Bedeschi, A. Tetrahedron Lett. 1997, 38, 2307-2310). ¹H NMR (300 MHz, DMSO-d₆) δ 11.94 (1H, s), 8.25 (1H, s), 7.88 (2H, d, J=7.5 Hz), 7.75 (1H, d, J=8.3 Hz), 7.50-7.48 (2H, m), 7.37-7.34 (1H, m), 7.06 (1H, s), 3.85 (3H, s). ¹³C NMR (75 MHz, DMSO-d₆) δ 167.2, 139.7, 139.5, 131.6, 129.0, 128.2, 127.9, 125.2, 122.6, 122.5, 120.9, 111.2, 99.9, 51.7.

### Example 2bb

### Synthesis of 4-Benzyloxy-5-methoxy-2-phenyl-1H-indole

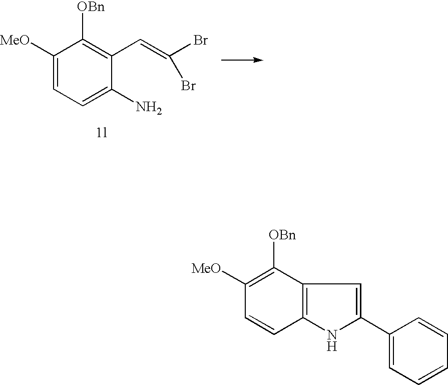

Following General Procedure A of Example 2a, a mixture of 3-benzyloxy-2-(2,2-dibromo-vinyl)-4-methoxy-phenylamine (0.124 g, 0.30 mmol), PhB(OH)₂ (0.055 g, 0.45 mmol), K₃PO₄.H₂O (0.35 g, 1.5 mmol), and catalyst solution (Pd(OAc)₂ (2.2 mg, 3 mol %) and s-Phos (8.8 mg, 6 mol %) in PhMe (1.5 mL)) was heated at 100° C. for 2 h. After an aqueous workup, the crude was purified by flash chromatography on silica gel (20% EtOAc in hexanes) to afford an off-white crystalline solid (0.0713 g, 72%). R_{f}=0.18 (2.5% EtOAc in hexanes). mp 106-108° C. IR (neat, cm⁻¹) 3425 (s), 3353 (s), 2935 (s), 1504 (s), 1484 (s), 1456 (s), 1329 (s), 1237 (s), 1092 (s). ¹H NMR (400 MHz, CDCl₃) δ 8.24 (1H, br), 7.57 (2H, d, J=7.7 Hz), 7.53 (2H, d, J=7.4 Hz), 7.40-7.34 (4H, m), 7.31-7.26 (2H, m), 7.01 (1H, d, J=8.8 Hz), 6.09 (1H, d, J=8.6 Hz), 6.80 (1H, d, J=1.3 Hz), 5.26 (2H, s), 3.87 (3H, s). ¹³C NMR (100 MHz, CDCl₃) δ 145.6, 141.1, 138.5, 138.5, 134.2, 132.4, 129.1, 128.5, 128.3, 128.0, 127.9, 125.3, 124.8, 112.3, 106.4, 97.5, 75.3, 58.5. HRMS calc'd for C22H19NO2 ([M]+) 329.1416. Found: 329.1423.

### Example 2cc

### Synthesis of 6-Benzyloxy-2-phenyl-1H-indole

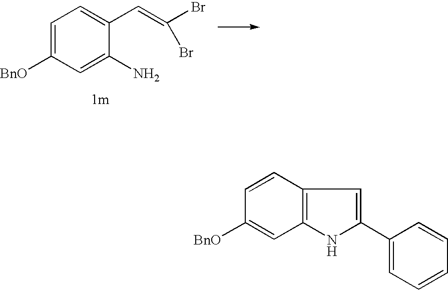

Following General Procedure A of Example 2a, a mixture of 5-benzyloxy-2-(2,2-dibromo-vinyl)-phenylamine (0.115 g, 0.30 mmol), PhB(OH)₂ (0.055 g, 0.45 mmol), K₃PO₄.H₂O (0.35 g, 1.5 mmol), and catalyst solution (Pd(OAc)₂ (3.4 mg, 5 mol %) and s-Phos (12.3 mg, 10 mol %) in PhMe (1.5 mL)) was heated at 100° C. for 1.5 h. After an aqueous workup, the crude was purified by flash chromatography on silica gel (20% EtOAc in hexanes) to afford an off-white crystalline solid (0.066 g, 73%). R_{f}=0.25 (2.5% EtOAc in hexanes). mp 200-202° C. (Lit: 202-204° C.). ¹H NMR (400 MHz, CDCl₃) δ 8.20 (1H, br), 7.62-7.60 (2H, m), 7.51-7.27 (9H, m), 6.95 (1H, d, J=2.2 Hz), 6.88 (1H, dd, J=8.6, 1.2 Hz), 6.75 (1H, d, J=1.3 Hz), 5.13 (2H, s). This compound was prepared previously in the prior art: Izumi, T.; Yokota, T. J. Heterocycl. Chem. 1992, 29, 1085-1090.

Preparation of 2,3-, 2,7- and 2,6,7-Substituted Indoles

The results of the preparation of various 2,3-, 2,7-, and 2,6,7-substituted indoles of Table 1 above are shown in Examples 2dd-2ii below.

### Example 2dd

### Synthesis of 2-Phenyl-3-trifluoromethyl-1H-indole

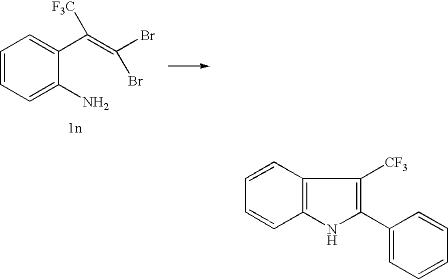

Following General Procedure A of Example 2a, a mixture of 2-(2,2-dibromo-1-trifluoromethyl-vinyl)-phenylamine (0.103 g, 0.30 mmol), PhB(OH)₂ (0.055 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 1 h. After an aqueous workup, the crude was purified by flash chromatography on silica gel (15% EtOAc in hexanes) to afford a yellowish crystalline solid (0.062 g, 79%). ¹H NMR (400 MHz, CDCl₃) δ 8.32 (1H, br), 7.82 (1H, d, J=7.5 Hz), 7.59-7.58 (2H, m), 7.48-7.46 (3H, m), 7.39 (1H, d, J=7.5 Hz), 7.30-7.23 (2H, m). ¹⁹F NMR (376 MHz, CDCl₃)-52.9 Hz. This compound was previously prepared in the literature (Mikami, K.; Matsumoto, Y.; Shiono, T. Science of Synthesis 2003, 2, 457-679).

### Example 2ee

### Synthesis of 3-(4-Fluoro-phenyl)-2-phenyl-1H-indole

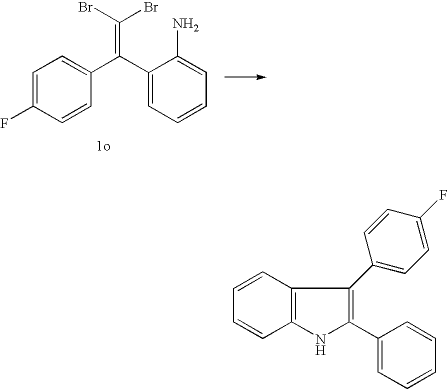

Following General Procedure A of Example 2a, a mixture of 2,2-dibromo-1-(4-fluorophenyl)-1-(2-nitrophenyl)ethene (0.125 g, 0.337 mmol), PhB(OH)₂ (0.062 g, 0.505 mmol), K₃PO₄.H₂O (0.35 g, 1.5 mmol), and catalyst solution (Pd(OAc)₂ (2.2 mg, 3 mol %) and s-Phos (8.8 mg, 6 mol %) in PhMe (1.5 mL)) was heated at 100° C. for 2 h. After an aqueous workup, the crude was purified by flash chromatography on silica gel (10% EtOAc in hexanes) to afford an off-white crystalline solid (0.087 g, 90%). R_{f}=0.21 (10% EtOAc in hexanes). mp 143-145° C. IR (neat, cm⁻¹) 3411 (s), 3055 (m), 1601 (w), 1553 (w), 1510 (s), 1452 (s), 1327 (m), 1221 (s). ¹H NMR (400 MHz, CDCl₃) δ 8.19 (1H, br), 7.62 (1H, d, J=7.9 Hz), 7.42-7.29 (8H, m), 7.24 (1H, t, J=7.3 Hz), 7.15 (1H, t, J=7.5 Hz), 7.06 (2H, t, J=8.7 Hz). ¹³C NMR (100 MHz, CDCl₃) δ 161.8 (J_{CF}=245 Hz), 136.0, 134.4, 132.7, 131.8 (J_{CF}=8.4 Hz), 131.2 (J_{CF}=3.1 Hz), 129.0, 128.9, 128.3, 128.0, 123.0, 120.7, 119.7, 115.7 (J_{CF}=21.5 Hz), 114.2, 111.1. ¹⁹F NMR (376 MHz, CDCl₃) δ −116.4 (1F, dddd, J_{FH}=7.9, 7.9, 5.3, 5.3 Hz). HRMS calc'd for C₂₀H₁₄NF ([M]⁺) 287.1110. Found: 287.1113.

### Example 2ff

### Synthesis of 2-Phenyl-3-methyl-1H-indole

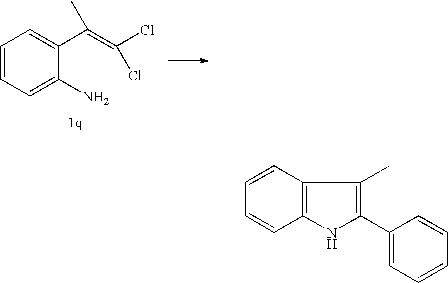

Following General Procedure A of Example 2a, a mixture of 2-(2,2-dichloro-1-methyl-vinyl)-phenylamine (0.101 g, 0.50 mmol), PhB(OH)₂ (0.092 g, 0.75 mmol), K₃PO₄.H₂O (0.58 g, 2.5 mmol), and catalyst solution (Pd(OAc)₂ (2.2 mg, 2 mol %) and s-Phos (8.1 mg, 4 mol %) in PhMe (2.5 mL)) was heated at 100° C. for 2 h. After an aqueous workup, the crude was purified by flash chromatography on silica gel (10% EtOAc in hexanes) to afford a white crystalline solid (0.099 g, 96%). This compound was prepared previously in the prior art (Izumi, T.; Yokota, T. J. Heterocycl. Chem. 1992, 29, 1085-1090).

### Example 2gg

### Synthesis of 2-Phenyl-3-phenylethynyl-1H-indole

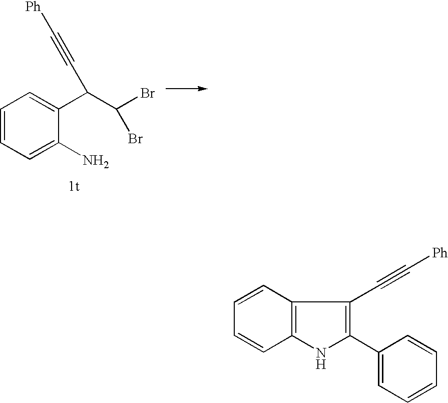

Following General Procedure A of Example 2a, a mixture of 2-(1-dibromomethylene-3-phenyl-prop-2-ynyl)-phenylamine (0.113 g, 0.30 mmol), PhB(OH)₂ (0.055 g, 0.45 mmol), K₃PO₄.H₂O (0.35 g, 1.5 mmol), and catalyst solution (Pd(OAc)₂ (2.2 mg, 2 mol %) and s-Phos (8.1 mg, 4 mol %) in PhMe (1.5 mL)) was heated at 100° C. for 2 h. After an aqueous workup, the crude was purified by flash chromatography on silica gel (10% EtOAc in hexanes) to afford a white crystalline solid (0.068 g, 77%). This compound was prepared previously in the prior art (Arcadi, A et al. J. Org. Chem. 2005, 70, 6213-6217).

### Example 2hh

### Synthesis of 7-Methyl-2-phenyl-1H-indole

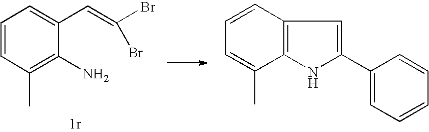

Following General Procedure A of Example 2a, a mixture of 2-(2,2-dibromo-vinyl)-6-methyl-phenylamine (0.087 g, 0.30 mmol), PhB(OH)₂ (0.055 g, 0.45 mmol), K₃PO₄.H₂O (0.35 g, 1.5 mmol), and catalyst solution (Pd(OAc)₂ (2.2 mg, 2 mol %) and s-Phos (8.1 mg, 4 mol %) in PhMe (1.5 mL)) was heated at 100° C. for 2 h. After an aqueous workup, the crude was purified by flash chromatography on silica gel (7.5% EtOAc in hexanes) to afford a white crystalline solid (0.0553 g, 89%). This compound was prepared previously in the prior art (Junjappa, H. Synthesis 1975, 798).

### Example 2ii

### Synthesis of 2-Phenyl-1H-benzo[g]indole

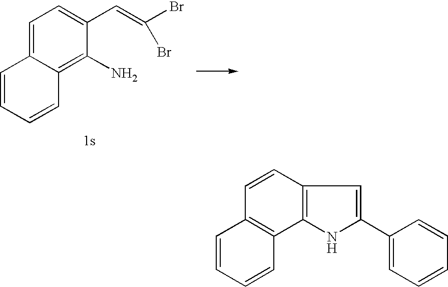

Following General Procedure A of Example 2a, a mixture of 2-(2,2-dibromo-vinyl)-naphthalen-1-ylamine (0.098 g, 0.30 mmol), PhB(OH)₂ (0.055 g, 0.45 mmol), K₃PO₄.H₂O (0.35 g, 1.5 mmol), and catalyst solution (Pd(OAc)₂ (2.2 mg, 2 mol %) and s-Phos (8.1 mg, 4 mol %) in PhMe (1.5 mL)) was heated at 100° C. for 2 h. After an aqueous workup, the crude was purified by flash chromatography on silica gel (7.5% EtOAc in hexanes) to afford a slightly yellow crystalline solid (0.0564 g, 77%). This compound was prepared previously in the prior art (Wagwa, S. et. al. J. Am. Chem. Soc. 1999, 121, 10251-10263.)

Preparation of N-Arylanilines

The results of the preparation of various N-arylaniline compounds of Tables 3 and Table 4 above are shown in Examples 3a-31 below.

### Example 3a

### General Procedure for Copper-Mediated Oxidative Coupling of Aniline and Boronic Acids—Synthesis of [2-(2,2-Dibromo-vinyl)-phenyl]-phenyl-amine

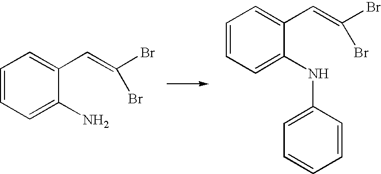

To a tube (24×150 mm) of Carousel reaction station was charged with 2-(2,2-dibromo-vinyl)-phenylamine (0.277 g, 1 mmol), PhB(OH)₂ (0.244 g, 2 mmol), Cu(OAc)₂ (0.182 g, 1 mmol), myristic acid (0.092 g, 0.4 mmol) and 2,6-lutidine (125 μL, 1.07 mmol) and toluene (3 mL). The mixture was stirred at 40° C. under O₂ atmosphere for 21 h. The mixture was diluted with Et₂O (10 mL) and Et₃N (1.5 mL), stirred at rt for 15 min and filtered through a short silica gel column, eluted with copious amount of Et₂O (˜30 mL). The product was further purified by flash chromatography on silica gel (2.5% EtOAc in hexanes) to afford the desired product as a solid (0.3134 g, 89%). R_{f}=0.28 (5% EtOAc in hexanes). mp 75-77° C. IR (neat, cm⁻¹) 3407 (m), 3035 (w), 1597 (m) 1577 (m), 1506 (s), 1455 (s), 1311 (s), 1214 (s). ¹H NMR (400 MHz, CDCl₃) δ 7.59 (1H, d, J=7.7 Hz), 7.39 (1H, s), 7.30-7.23 (4H, m), 7.03-6.94 (4H, 4m), 5.47 (1H, s). ¹³C NMR (100 MHz, CDCl₃) δ 143.0, 141.0, 134.3, 129.9, 129.7, 129.6, 126.4, 121.8, 121.4, 118.7, 118.2, 93.1. HRMS calc'd for C₁₄H₁₁NBr₂ ([M]⁺) 350.9258. Found: 350.9253.

### Example 3b

### Synthesis of [2-(2,2-Dibromo-vinyl)-phenyl]-(4-fluoro-phenyl)-amine

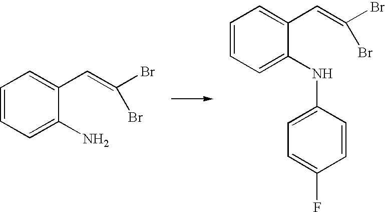

Following the general procedure of Example 3a for copper-mediated coupling reaction starting with 2-(2,2-dibromo-vinyl)-phenylamine (0.277 g, 1 mmol), ArB(OH)₂ (0.280 g, 2 mmol), Cu(OAc)₂ (0.182 g, 1 mmol), myristic acid (0.092 g, 0.4 mmol) and 2,6-lutidine (125 μL, 1.07 mmol) in toluene (3 mL). The mixture was stirred at 40° C. for 21 h and 60° C. for 6 h under O₂ atmosphere. General workup procedure was also followed. The product was further purified by flash chromatography on silica gel (2.5% EtOAc in hexanes) to afford the desired product as a solid (0.268 g, 72%). R_{f}=0.27 (5% EtOAc in hexanes). mp 75-77° C. IR (neat, cm⁻¹) 3407 (m), 3035 (m), 1597 (m), 1577 (m), 1506 (s), 1455 (s), 1311 (s), 1214 (s). ¹H NMR (400 MHz, CDCl₃) δ 7.46 (1H, d, J=7.7 Hz), 7.38 (1H, s), 7.23 (1H, ddd, J=8.0, 8.0, 1.1 Hz), 7.10 (1H, d, J=8.1 Hz), 7.01-6.99 (4H, m), 6.95 (1H, t, J=7.8 Hz), 5.38 (1H, s). ¹³C NMR (100 MHz, CDCl₃) δ 158.6 (J_{CF}=241 Hz), 141.7, 138.8 (J_{CF}=2.3 Hz), 134.2, 129.9, 129.8, 125.6, 121.5 (J_{CF}=8.4 Hz), 120.9, 116.9, 116.2 (J_{CF}=22.2 Hz), 93.3. ¹⁹F NMR (282 MHz, CDCl₃) δ −121.1 (1F, dddd, J_{FH}=7.0, 7.0, 6.0, 6.0 Hz). HRMS calc'd for C₁₄H₁₀NFBr₂ ([M]⁺) 368.9164. Found: 368.9175.

### Example 3c

### Synthesis of [2-(2,2-Dibromo-vinyl)-phenyl]-(4-trifluoromethyl-phenyl)-amine

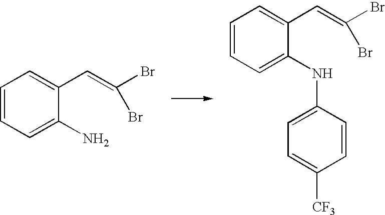

Following the general procedure for copper-mediated coupling reaction of Example 3a, starting with 2-(2,2-dibromo-vinyl)-phenylamine (0.281 g, 1.01 mmol), ArB(OH)₂ (0.360 g, 2 mmol), Cu(OAc)₂ (0.182 g, 1 mmol), myristic acid (0.092 g, 0.4 mmol) and 2,6-lutidine (125 μL, 1.07 mmol) in toluene (3 mL). The mixture was stirred at 40° C. for 21 h and 60° C. for 3 h under O₂ atmosphere. General workup procedure was also followed. The product was further purified by flash chromatography on silica gel (2.5% EtOAc in hexanes) to afford the desired product as a solid (0.352 g, 84%). R_{f}=0.15 (2.5% EtOAc in hexanes). mp 74-75° C. IR (neat, cm⁻¹) 3405 (m), 1616 (m), 1597 (m), 1524 (m), 1323 (s), 1162 (m), 1113 (s), 1066 (m). ¹H NMR (400 MHz, CDCl₃) δ 7.56 (1H, d, J=7.7 Hz), 7.48 (2H, d, J=8.6 Hz), 7.38 (1H, s), 7.33-7.32 (2H, apparent d), 7.25-7.10 (1H, m), 6.97 (1H, d, J=8.4 Hz), 5.67 (1H, s). ¹³C NMR (75 MHz, CDCl₃) δ 146.7, 139.0, 134.0, 130.2, 129.8, 128.7, 126.9 (q, J_{CF}=3.7 Hz), 124.7 (J_{CF}=269 Hz), 123.5, 122.4 (J_{CF}=32.5 Hz), 121.0, 116.2, 93.5. ¹⁹F NMR (376 MHz, CDCl₃) δ −61.5 (s). HRMS calc'd for C₁₅H₁₀NF₃Br₂ ([M]⁺) 418.9132. Found: 418.9147.

### Example 3d

### Synthesis of [2-(2,2-Dibromo-vinyl)-phenyl]-(3,4-dimethoxy-phenyl)-amine

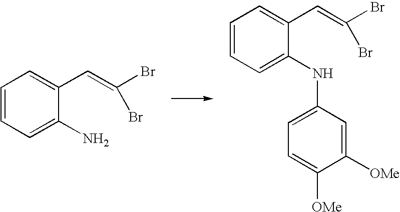

Following the General Procedure for copper-mediated coupling reaction of example 3a, starting with 2-(2,2-dibromo-vinyl)-phenylamine (0.281 g, 1.01 mmol), ArB(OH)₂ (0.364 g, 2 mmol), Cu(OAc)₂ (0.182 g, 1 mmol), myristic acid (0.092 g, 0.4 mmol) and 2,6-lutidine (125 μL, 1.07 mmol) in toluene (3 mL). The mixture was stirred at 40° C. for 21 h under O₂ atmosphere. General workup procedure was also followed. The product was further purified by flash chromatography on silica gel (15% EtOAc in hexanes) to afford the desired product as a solid (0.233 g, 56%). R_{f}=0.20 (20% EtOAc in hexanes). mp 83-85° C. IR (neat, cm⁻¹) 3354 (m), 2932 (w), 1597 (m), 1513 (s), 1454 (s), 1253 (s), 1231 (s), 1026 (m). ¹H NMR (300 MHz, CDCl₃) δ 7.44 (1H, d, J=7.8 Hz), 7.39 (1H, s), 7.20 (1H, ddd, J=7.8, 7.8, 1.3 Hz), 7.07 (1H, dd, J=8.3, 0.9 Hz), 6.89 (1H, ddd, J=7.4, 7.4, 1.1 Hz), 6.81 (1H, d, J=8.2 Hz), 6.66-6.61 (2H, m), 5.37 (1H, s), 3.86 (3H, s), 3.82 (3H, s). ¹³C NMR (75 MHz, CDCl₃) δ 149.8, 145.2, 142.5, 136.0, 134.2, 129.7, 129.7, 124.6, 120.0, 116.1, 112.9, 112.3, 105.9, 93.0, 56.4, 56.1. HRMS (ESI) calc'd for C₁₆H₁₆NO₂Br₂ ([MH]⁺) 411.9542. Found: 411.9529.

### Example 3e

### Synthesis of [2-(2,2-Dibromo-vinyl)-phenyl]-o-tolyl-amine

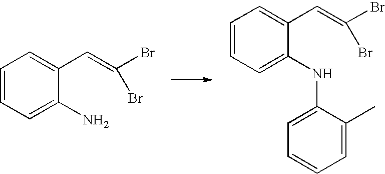

Following the General Procedure for copper-mediated coupling reaction of Example 3a, starting with 2-(2,2-dibromo-vinyl)-phenylamine (0.280 g, 1.01 mmol), ArB(OH)₂ (0.272 g, 2 mmol), Cu(OAc)₂ (0.182 g, 1 mmol), myristic acid (0.092 g, 0.4 mmol) and 2,6-lutidine (125 μL, 1.07 mmol) in toluene (3 mL). The mixture was stirred at 40° C. for 21 h and 60° C. for 4 h under O₂ atmosphere. General workup procedure was also followed. The product was further purified by flash chromatography on silica gel (2.5% EtOAc in hexanes) to afford the desired product as a solid (0.255 g, 69%). R_{f}=0.38 (2.5% EtOAc in hexanes). mp 65-67° C. IR (neat, cm⁻¹) 3391 (m), 2924 (m), 1585 (s), 1504 (s), 1455 (s), 1307 (s). ¹H NMR (400 MHz, CDCl₃) δ 7.46 (1H, dd, J==7.3, 0.9 Hz), 7.41 (1H, s), 7.24-7.20 (2H, m), 7.14 (1H, t, J=7.6 Hz), 7.07 (1H, d, J=7.0 Hz), 7.00-6.92 (3H, m), 5.23 (1H, s), 2.24 (3H, s). ¹³C NMR (75 MHz, CDCl₃) δ 141.8, 141.0, 134.4, 131.2, 129.7, 129.3, 127.1, 125.6, 122.9, 120.7, 120.2, 117.7, 93.2, 18.1. HRMS calc'd for C₁₅H₁₃NBr₂ ([M]⁺) 364.9415. Found: 364.9420.

### Example 3f

### Synthesis of [2-(2,2-Dichloro-1-methyl-vinyl)-phenyl]-phenyl-amine

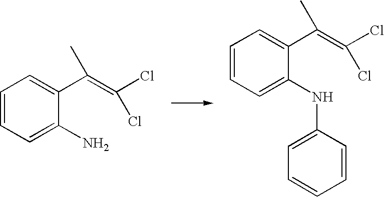

Following the General Procedure for copper-mediated coupling reaction of example 3a, starting with 2-(2,2-dichloro-1-methyl-vinyl)-phenylamine (0.105 g, 0.52 mmol), ArB(OH)₂ (0.122 g, 1 mmol), Cu(OAc)₂ (0.091 g, 0.5 mmol), myristic acid (0.046 g, 0.2 mmol) and 2,6-lutidine (62.5 μL, 0.54 mmol) in toluene (1.5 mL). The mixture was stirred at 40° C. for 6.5 h under O₂ atmosphere. General workup procedure was also followed. The product was further purified by flash chromatography on silica gel (2.5% EtOAc in hexanes) to afford the desired product as an oil (0.1415 g, 98%). R_{f}=0.20 (2.5% EtOAc in hexanes). IR (neat, cm⁻¹) 3411 (m), 3046 (m), 1593 (s), 1505 (s), 1451 (m), 1308 (s). ¹H NMR (400 MHz, CDCl₃) δ 7.30-7.25 (3H, m), 7.21 (1H, ddd, J=7.6, 7.6, 1.7 Hz), 7.10 (1H, dd, J=7.6, 1.6), 7.07-7.04 (2H, m), 6.98-6.93 (2H, m), 5.45 (1H, s), 2.14 (3H, s). ¹³C NMR (75 MHz, CDCl₃) δ 143.2, 139.9, 134.2, 130.2, 129.6, 129.1, 129.0, 121.7, 121.4, 118.9, 118.8, 118.0, 22.2. HRMS calc'd for C₁₅H₁₃NCl₂ ([M]⁺) 277.0425. Found: 277.0426.

### Example 3g

### Synthesis of [2-(2,2-Dichloro-1-methyl-vinyl)-phenyl]-(4-fluoro-phenyl)-amine

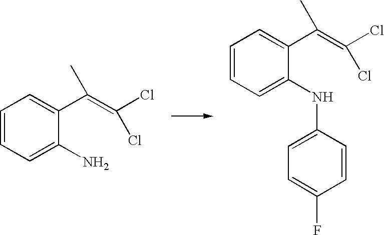

Following the General Procedure for copper-mediated coupling reaction of example 3a, starting with 2-(2,2-dichloro-1-methyl-vinyl)-phenylamine (0.200 g, 1 mmol), ArB(OH)₂ (0.280 g, 2 mmol), Cu(OAc)₂ (0.182 g, 1 mmol), myristic acid (0.092 g, 0.4 mmol) and 2,6-lutidine (125 μL, 1.07 mmol) in toluene (3 mL). The mixture was stirred at 40° C. for 35 h under O₂ atmosphere. General workup procedure was also followed. The product was further purified by flash chromatography on silica gel (2.5% EtOAc in hexanes) to afford the desired product as an oil (0.193 g, 65%) and starting aniline (0.061 g, 30%). R_{f}=0.25 (5% EtOAc in hexanes). IR (neat, cm⁻¹) 3415 (m), 2923 (w), 1598 (m), 1580 (m), 1509 (s), 1451 (m), 1309 (m), 1217 (s). ¹H NMR (400 MHz, CDCl₃) δ 7.19 (1H, ddd, J=7.7, 7.7, 1.5 Hz), 7.11-6.96 (6H, m), 6.92 (1H, ddd, J=7.4, 7.4, 1.2 Hz), 5.35 (1H, s), 2.15 (3H, s). ¹³C NMR (100 MHz, CDCl₃) δ 159.1 (J_{CF}=241 Hz), 140.8, 138.9 (J_{CF}=2.6 Hz), 134.0, 129.2, 129.1, 129.0, 122.9 (J_{CF}=7.8 Hz), 120.9, 119.0, 116.7, 116.2 (J_{CF}=22.4 Hz), 22.2. ¹⁹F NMR (376 MHz, CDCl₃) δ −121.2 (1F, dddd, J_{FH}=8.5, 8.5, 4.0, 4.0 Hz). HRMS calc'd for C₁₅H₁₂NFCl₂ ([M]⁺) 295.0331. Found: 295.0330.

### Example 3h

### Synthesis of [2-(2,2-Dichloro-1-methyl-vinyl)-phenyl]-(4-trifluoromethyl-phenyl)-amine

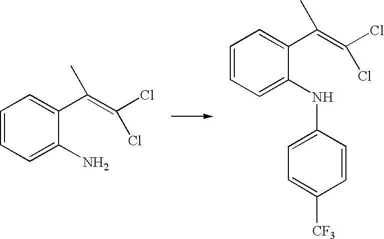

Following the general procedure for copper-mediated coupling reaction of example 3a, starting with 2-(2,2-dichloro-1-methyl-vinyl)-phenylamine (0.204 g, 1.01 mmol), ArB(OH)₂ (0.380 g, 2 mmol), Cu(OAc)₂ (0.363 g, 2 mmol), myristic acid (0.114 g, 0.5 mmol) and 2,6-lutidine (125 μL, 1.07 mmol) in toluene (3 mL). The mixture was stirred at rt for 18 h and 40° C. for 4 h under O₂ atmosphere. General workup procedure was also followed. The product was further purified by flash chromatography on silica gel (2.5% EtOAc in hexanes) to afford the desired product as an oil (0.240 g, 69%). R_{f}=0.32 (5% EtOAc in hexanes). IR (neat, cm⁻¹) 3418 (m), 3061 (w), 1615 (s), 1598 (s), 1578 (s), 1525 (s), 1505 (s), 1453 (s), 1318 (s), 1162 (s), 1112 (s), 1067 (s). ¹H NM (400 MHz, CDCl₃) δ 7.47 (2H, d, J=8.4 Hz), 7.37 (1H, dd, J=8.3, 1.2 Hz), 7.29 (1H, ddd, J=7.7, 7.7, 1.5 Hz), 7.17 (1H, dd, J=7.8, 1.4 Hz), 7.10 (1H, ddd, J=7.4, 7.4, 1.3 Hz), 7.01 (2H, d, J=8.4 Hz), 5.63 (1H, s), 2.10 (3H, s). ¹³C NMR (100 MHz, CDCl₃) δ 147.0, 138.0, 133.9, 132.7, 129.5, 129.2, 126.9 (q, J_{CF}=3.1 Hz), 124.8 (q, J_{CF}=271 Hz), 123.7, 122.3 (q, J_{CF}=32.2 Hz), 121.1, 119.0, 116.1, 22.3. ¹⁹F NMR (376 MHz, CDCl₃) δ −61.5 (s). HRMS calc'd for C₁₆H₁₂NF₃Cl₂ ([M]⁺) 345.0299. Found: 345.0297.

### Example 3i

### Synthesis of [2-(2,2-Dichloro-1-methyl-vinyl)-phenyl]-(3,4-dimethoxy-phenyl)-amine

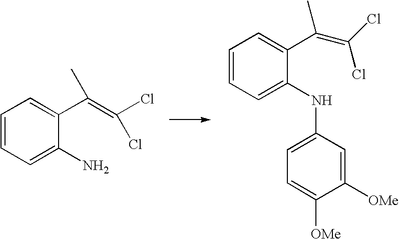

Following the General Procedure for copper-mediated coupling reaction of Example 3a, starting with 2-(2,2-dichloro-1-methyl-vinyl)-phenylamine (0.105 g, 0.52 mmol), ArB(OH)₂ (0.182 g, 1 mmol), Cu(OAc)₂ (0.091 g, 0.5 mmol), myristic acid (0.046 g, 0.2 mmol) and 2,6-lutidine (62.5 μL, 0.54 mmol) in toluene (1.5 mL). The mixture was stirred at 40° C. for 8 h under O₂ atmosphere. General workup procedure was also followed. The product was further purified by flash chromatography on silica gel (10→15% EtOAc in hexanes) to afford the desired product as a solid (0.1566 g, 89%). R_{f}=0.28 (20% EtOAc in hexanes). mp 94-96° C. IR (neat, cm⁻¹) 3367 (m), 2931 (m), 1597 (m), 1512 (s), 1449 (s), 1257 (s), 1232 (s), 1027 (m). ¹H NMR (300 MHz, CDCl₃) δ 7.16 (1H, ddd, J=7.8, 7.8, 1.4 Hz), 7.07 (1H, dd, J=8.2, 1.2 Hz), 7.05 (1H, dd, J=7.6, 1.6 Hz), 6.86 (1H, dd, J=7.4, 7.4, 1.2 Hz), 6.81 (1H, d, J=8.3 Hz), 6.69-6.66 (2H, m), 5.32 (1H, s), 3.86 (3H, s), 3.84 (3H, s), 2.17 (3H, s). ¹³C NMR (100 MHz, CDCl₃) δ 149.8, 145.3, 141.5, 136.1, 134.1, 129.0, 128.8, 128.2, 120.0, 118.8, 115.7, 113.5, 112.3, 106.5, 56.4, 56.1, 22.1. HRMS (ESI) calc'd for C₁₇H₁₈NO₂Cl₂ ([MH]⁺) 338.0709. Found: 338.0720.

### Example 3j

### Synthesis of 1-{4-[2-(2,2-Dichloro-1-methyl-vinyl)-phenylamino]-phenyl}-ethanone

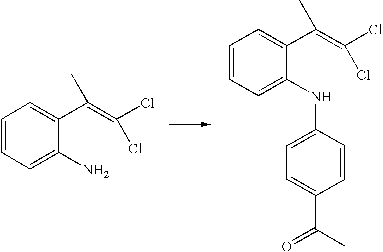

Following the General Procedure for copper-mediated coupling reaction of Example 3a, starting with 2-(2,2-dichloro-1-methyl-vinyl)-phenylamine (0.218 g, 1.08 mmol), ArB(OH)₂ (0.338 g, 2 mmol), Cu(OAc)₂ (0.273 g, 1.5 mmol), myristic acid (0.092 g, 0.4 mmol) and 2,6-lutidine (125 μL, 1.07 mmol) in toluene (3 mL). The mixture was stirred at 40° C. for 5 h and 60° C. for 5 h under O₂ atmosphere. General workup procedure was also followed. The product was further purified by flash chromatography on silica gel (10→15→20% EtOAc in hexanes) to afford the desired product as an solid (0.242 g, 70%). R_{f}=0.23 (20% EtOAc in hexanes). mp 81-82° C. IR (neat, cm⁻¹) 3324(m), 1661 (s), 1592(s), 1519(s), 1276(s), 1178. ¹H NMR (400 MHz, CDCl₃) δ 7.86 (2H, d, J=8.6 Hz), 7.40 (1H, d, J=7.7 Hz), 7.31 (1H, ddd, J=7.7, 7.7, 1.6 Hz), 7.19 (1H, dd, J=7.6, 1.6 Hz), 7.13 (1H, ddd, J=7.5, 7.5, 1.1 Hz), 6.95 (2H, d J=8.8 Hz), 5.87 (1H, s), 2.53 (3H, s), 2.09 (3H, s). ¹³C NMR (100 MHz, CDCl₃) δ 196.6, 148.7, 137.4, 133.9, 133.3, 130.8, 129.6, 129.5, 129.2, 124.2, 122.2, 119.0, 115.0, 26.4, 22.3. HRMS (ESI) calc'd for C₁₇H₁₆NOCl₂ ([MH]⁺) 320.0603. Found: 320.0598.

### Example 3k

### Synthesis of [2-(2,2-Dichloro-1-methyl-vinyl)-phenyl]-o-tolyl-amine

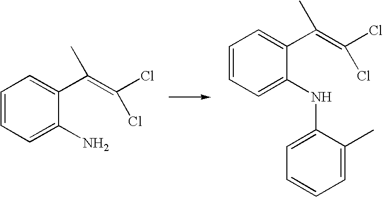

Following the General Procedure for copper-mediated coupling reaction of example 3a, starting with 2-(2,2-dichloro-1-methyl-vinyl)-phenylamine (0.210 g, 1.04 mmol), ArB(OH)₂ (0.272 g, 2 mmol), Cu(OAc)₂ (0.273 g, 1.5 mmol), myristic acid (0.092 g, 0.4 mmol) and 2,6-lutidine (125 μL, 1.07 mmol) in toluene (3 mL). The mixture was stirred at 40° C. for 13.5 h and 60° C. for 9 h under O₂ atmosphere. General workup procedure was also followed. The product was further purified by flash chromatography on silica gel (2.5% EtOAc in hexanes) to afford the desired product as a solid (0.213 g, 70%). R_{f}=0.35 (2.5% EtOAc in hexanes). mp 49-51° C. IR (neat, cm⁻¹) 3428 (m), 3034 (w), 2918 (w), 1584 (s), 1504 (s), 1452 (s), 1306 (s), 1026 (m). ¹H NMR (400 MHz, CDCl₃) δ 7.21 (1H, d, J=7.5 Hz), 7.18-7.14 (2H, m), 7.08 (1H, dd, J=7.6, 1.4 Hz), 7.00-6.96 (2H, m), 6.90 (1H, t, J=7.5 Hz), 5.28 (1H, s), 2.24 (3H, s), 2.16 (3H, s). ¹³C NMR (75 MHz, CDCl₃) δ 141.8, 141.0, 134.4, 131.2, 129.7, 129.3, 127.1, 125.6, 122.9, 120.7, 120.2, 117.7, 93.2, 18.1. HRMS calc'd for C₁₆H₁₅NCl₂ ([M]⁺) 291.0581. Found: 291.0588.

Preparation of N-aryl 2-Substituted Indoles

The results of the preparation of various N-arylindoles of Tables 3 and Table 4 above, having substituents at the 1, 2 and in some cases 3-positions of the indole ring are shown in Examples 4a-4k below.

### Example 4a

### Synthesis of 1,2-Diphenyl-1H-indole

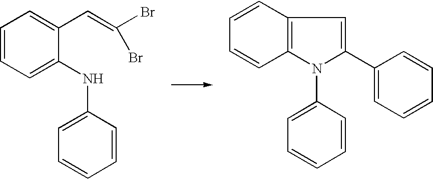

Following General Procedure A of Example 2a, a mixture of [2-(2,2-dibromo-vinyl)-phenyl]-phenyl-amine (0.108 g, 0.306 mmol), PhB(OH)₂ (0.055 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 1 h. After an aqueous workup, the crude was purified by flash chromatography on silica gel (2.5% EtOAc in hexanes) to afford a white solid (0.076 g, 92%); mp 78-80° C. (Lit. 81° C.) (Horrocks, D. L.; Wirth, H. O. J. Chem. Phys. 1967, 47, 3241-32471).

### Example 4b

### Synthesis of 2-(4-Fluoro-phenyl)-1-phenyl-1H-indole

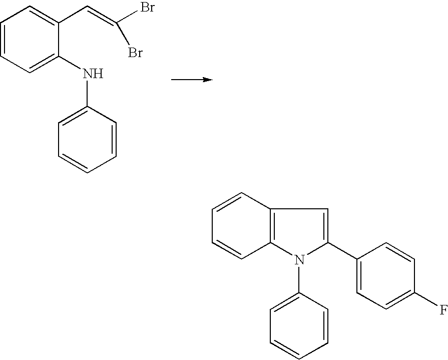

Following General Procedure A of Example 2a, a mixture of [2-(2,2-dibromo-vinyl)-phenyl]-phenyl-amine (0.109 g, 0.31 mmol), 4-FPhB(OH)₂ (0.065 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 2 h. After an aqueous workup, the crude was purified by flash chromatography on silica gel (2% EtOAc in hexanes) to afford a white solid (0.076 g, 86%). R_{f}=0.25 (2.5% EtOAc in hexanes). mp 121-122° C. (Lit: 123-124° C.) (Hay, A. S.; Paventi, M. In PCT Int. Appl. WO 93 09079, 1993) ¹H NMR (400 MHz, CDCl₃) δ 7.69-7.65 (1H, m), 7.25-7.16 (10H, m), 7.09 (2H, t, J=8.5 Hz), 6.79 (1H, s). ¹⁹F NMR (376 MHz, CDCl₃) δ −114.2 (1F, dddd, J_{FH}=7.9, 7.9, 5.3, 5.3 Hz).

### Example 4c

### Synthesis of 1-(4-Fluoro-phenyl)-2-phenyl-1H-indole

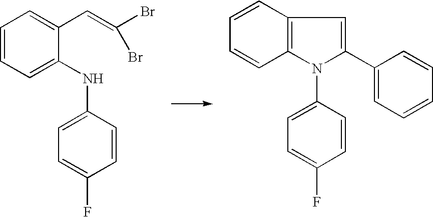

Following General Procedure A of Example 2a, a mixture of [2-(2,2-dibromo-vinyl)-phenyl]-phenyl-amine (0.111 g, 0.30 mmol), PhB(OH)₂ (0.055 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 1 h. After an aqueous workup, the crude was purified by flash chromatography on silica gel (2.5% EtOAc in hexanes) to afford a white solid (0.0775 g, 90%). R_{f}=0.20 (2.5% EtOAc in hexanes). mp 123-124° C. IR (neat, cm⁻¹) 3062 (m), 1601 (w), 1509 (s), 1457 (m), 1324 (w), 1222 (m). ¹H NMR (400 MHz, CDCl₃) δ 7.69-7.65 (1H, m), 7.25-7.16 (10H, m), 7.09 (2H, t, J=8.5 Hz), 6.79 (1H, s). ¹³C NMR (100 MHz, CDCl₃) δ 161.7 (J_{CF}=248 Hz), 141.0, 139.3, 134.8 (J_{CF}=3.1 Hz), 132.5, 129.9 (J_{CF}=8.4 Hz), 129.1, 128.5, 128.4, 127.7, 122.7, 121.0, 120.8, 116.4 (J_{CF}=23.0 Hz), 110.6, 104.0. ¹⁹F NMR (376 MHz, CDCl₃) δ −114.2 (1F, dddd, J_{FH}=7.9, 7.9, 5.3, 5.3 Hz). HRMS calc'd for C₂₀H₁₄NF ([M]⁺) 287.1110. Found: 287.1115.

### Example 4d

### Synthesis of 1-(3,4-Dimethoxy-phenyl)-2-(4-trifluoromethyl-phenyl)-1H-indole

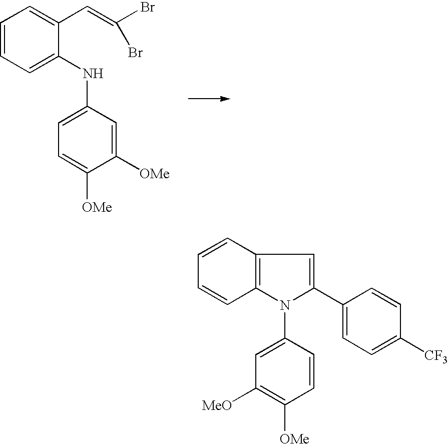

Following General Procedure A of Example 2a, a mixture of [2-(2,2-dibromo-vinyl)-phenyl]-(3,4-dimethoxy-phenyl)-amine (0.124 g, 0.30 mmol), 4-CF₃PhB(OH)₂ (0.083 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 2.5 h. After an aqueous workup, the crude was purified by flash chromatography on silica gel (15→20% EtOAc in hexanes) to afford a white solid (0.097 g, 81%). R_{f}=0.22 (20% EtOAc in hexanes). mp 190-191° C. IR (neat, cm⁻¹) 2921 (w), 1612 (m), 1514 (m), 1451 (m), 1322 (s), 1110 (s). ¹H NMR (400 MHz, CDCl₃) δ 7.71-7.68 (1H, m), 7.50 (2H, d, J=8.3 Hz), 7.40 (2H, d, J=8.1 Hz), 7.28 (1H, d, J=7.5 Hz), 7.23-7.17 (2H, m), 6.91 (1H, d, J=8.6 Hz), 6.87 (1H, s), 6.83 (1H, dd, J=8.5, 2.3 Hz), 6.74 (1H, d, J=2.2 Hz), 3.94 (3H, s), 3.73 (3H, s). ¹³C NMR (100 MHz, CDCl₃) δ 149.7, 148.6, 139.8, 139.2, 136.3, 131.2, 129.2 (q, J_{CF}=32.7 Hz), 128.9, 125.4 (q, J_{CF}=3.6 Hz), 124.3 (q, J_{CF}=272 Hz), 123.2, 121.1, 121.0, 120.5, 111.7, 111.5, 111.0, 104.6, 56.2. HRMS calc'd for C₂₃H₁₈NO₂F₃ ([M]⁺) 397.1290. Found: 397.1269.

### Example 4e

### Synthesis of 2-(2-Fluoro-phenyl)-1-(4-trifluoromethyl-phenyl)-1H-indole

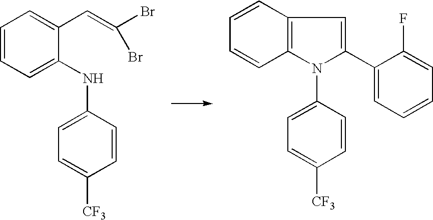

Following General Procedure A of Example 2a, a mixture of [2-(2,2-dibromo-vinyl)-phenyl]-(4-trifluoromethyl-phenyl)-amine (0.126 g, 0.30 mmol), 2-FPhB(OH)₂ (0.063 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 2.5 h. After an aqueous workup, the crude was purified by flash chromatography on silica gel (2.5% EtOAc in hexanes) to afford a white solid (0.0873 g, 82%). R_{f}=0.22 (2.5% EtOAc in hexanes). mp 92-93° C. IR (neat, cm⁻¹) 3061 (w), 1615 (m), 1452 (m), 1324 (s), 1168 (s), 1127 (s). ¹H NMR (400 MHz, CDCl₃) δ 7.72-7.70 (1H, m), 7.63 (2H, d, J=8.3 Hz), 7.36-7.33 (3H, m), 7.31-7.27 (2H, m), 7.26-7.21 (2H, m), 7.10 (1H, ddd, J=7.8, 7.3, 1.1 Hz), 6.98 (1H, ddd, J=10.0, 8.6, 1.3 Hz), 6.85 (1H, s). ¹³C NMR (100 MHz, CDCl₃) δ 159.9 (d, J_{CF}=250 Hz), 141.8, 138.3, 134.6, 132.0 (d, J_{CF}=3.1 Hz), 130.4 (d, J_{CF}=7.5 Hz), 129.1 (q, J_{CF}=33 Hz), 128.6, 127.6, 126.5 (q, J_{CF}=3.8 Hz), 124.4 (d, J_{CF}=3.8 Hz), 124.1 (q, J_{CF}=272 Hz), 123.3, 121.4, 121.2, 120.6 (d, J_{CF}=14.6 Hz), 116.2 (d, J_{CF}=22.2 Hz), 110.5, 106.7 (d, J_{CF}=2.3 Hz). ¹⁹F NMR (376 MHz, CDCl₃) δ −62.4 (3F, s), −112.6 (1F, ddd, J_{FH}=10.1, 7.2, 4.3 Hz). HRMS calc'd for C₂₁H₁₃NF₄ ([M]⁺) 355.0984. Found: 355.0999.

### Example 4f

### Synthesis of 1-(4-Fluoro-phenyl)-3-methyl-2-phenyl-1H-indole

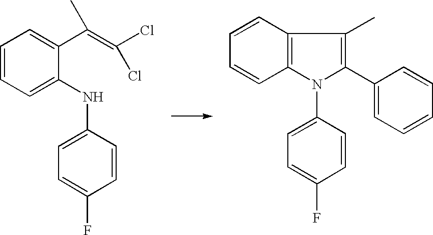

Following General Procedure A of Example 2a, a mixture of [2-(2,2-dichloro-1-methyl-vinyl)-phenyl]-(4-fluoro-phenyl)-amine (0.088 g, 0.297 mmol), PhB(OH)₂ (0.055 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 1 h. After an aqueous workup, the crude was purified by flash chromatography on silica gel (2.5% EtOAc in hexanes) to afford a white solid (0.0838 g, 94%). R_{f}=0.28 (2.5% EtOAc in hexanes). mp 108-110° C. IR (neat, cm⁻¹) 3053 (w), 2916 (w), 1603 (w), 1510 (s), 1457 (m), 1364 (w), 1217 (m). ¹H NMR (400 MHz, CDCl₃) δ 7.68-7.64 (1H, m), 7.31-7.18 (8H, m), 7.15-7.11 (2H, m), 7.02 (2H, t, J=7.5 Hz), 2.40 (3H, s). ¹³C NMR (100 MHz, CDCl₃) δ 161.3 (J_{CF}=246 Hz), 137.9, 137.2, 134.9 (J_{CF}=3.2 Hz), 132.1, 130.8, 129.6 (J_{CF}=8.3 Hz), 129.2, 128.3, 127.5, 122.8, 120.4, 119.2, 116.2 (J_{CF}=22.7 Hz), 111.0, 110.3, 9.8. ¹⁹F NMR (376 MHz, CDCl₃) δ −115.0 (1F, dddd, J_{FH}=7.9, 7.9, 5.3., 5.3 Hz). HRMS calc'd for C₂₁H₁₆NF ([M]⁺) 301.1267. Found: 301.1260. Anal. Calc'd for C₂₁H₁₆NF: C, 83.70; H, 5.35; N, 4.65. Found: C, 83.91; H, 5.26; N, 4.64.

### Example 4g

### Synthesis of 3-Methyl-2-phenyl-1-o-tolyl-1H-indole

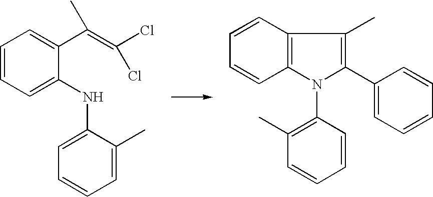

Following General Procedure A of Example 2a, a mixture of [2-(2,2-dichloro-1-methyl-vinyl)-phenyl]-o-tolyl-amine (0.088 g, 0.30 mmol), PhB(OH)₂ (0.055 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 2.5 h. After an aqueous workup, the crude was purified by flash chromatography on silica gel (2.5% EtOAc in hexanes) to afford a white solid (0.0684 g, 77%). R_{f}=0.28 (2.5% EtOAc in hexanes). mp 106-107° C. IR (neat, cm⁻¹) 3051 (w), 2917 (w), 1603 (w), 1493 (s), 1457 (s), 1359 (s), 1225 (m). ¹H NMR (400 MHz, CDCl₃) δ 7.69-7.64 (1H, m), 7.26-7.14 (11H, m), 6.94-6.90 (1H, m), 2.43 (3H, s), 1.88 (3H, s). ¹³C NMR (100 MHz, CDCl₃) δ 137.8, 137.8, 137.7, 137.1, 132.3, 131.1, 130.4, 130.0, 129.1, 128.2, 128.1, 127.3, 126.7, 122.5, 119.9, 119.0, 110.7, 109.9, 17.9, 9.9. HRMS calc'd for C₂₂H₁₉N ([M]⁺) 297.1518. Found: 297.1511.

### Example 4h

### Synthesis of 2-(4-Methoxy-phenyl)-3-methyl-1-(4-trifluoromethyl-phenyl)-1H-indole

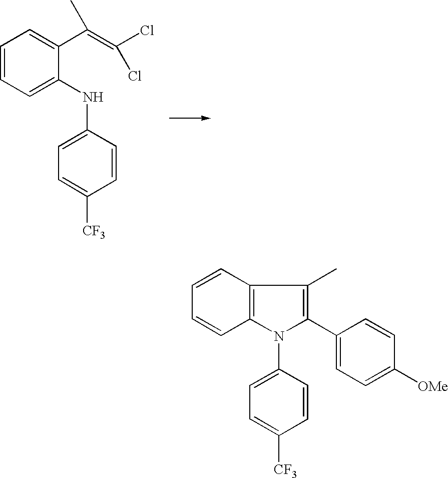

Following General Procedure A of Example 2a, a mixture of [2-(2,2-dichloro-1-methyl-vinyl)-phenyl]-(4-trifluoromethyl-phenyl)-amine (0.106 g, 0.306 mmol), 4-MeOPhB(OH)₂ (0.068 g, 0.45 mmol), K₃PO₄.H₂O (0.35 g, 1.5 mmol), and catalyst solution (Pd(OAc)₂ (2.2 mg, 3 mol %) and s-Phos (8.1 mg, 6 mol %) in PhMe (1.5 mL)) was heated at 100° C. for 11 h. After an aqueous workup, the crude was purified by flash chromatography on silica gel (2.5% EtOAc in hexanes) to afford a white solid (0.092 g, 79%). R_{f}=0.22 (2.5% EtOAc in hexanes). mp 124-125° C. IR (neat, cm⁻¹) 3052 (w), 2935 (w), 1613 (m), 1509 (m), 1456 (m), 1363 (m), 1323 (s), 1249 (s), 1173 (s), 1126 (s), 1067 (m). ¹H NMR (400 MHz, CDCl₃) δ 7.66-7.63 (1H, m), 7.59 (2H, d, J=8.1 Hz), 7.34-7.31 (1H, m), 7.26 (2H, d, J=8.3 Hz), 7.22-7.18 (2H, m), 7.12-7.08 (2H, m), 6.85-6.82 (2H, m), 3.79 (3H, s), 2.37 (3H, s). ¹³C NMR (100 MHz, CDCl₃) δ 158.9, 142.0, 137.0, 136.4, 131.7, 129.5, 128.2 (q, J_{CF}=32.2 Hz), 127.7, 126.2 (q, J_{CF}=3.6 Hz), 124.0 (q, J_{CF}=272 Hz), 123.9, 122.7, 120.6, 119.0, 113.8, 111.4, 109.9, 55.2, 9.5. ¹⁹F NMR (376 MHz, CDCl₃) δ −62.3 (s). HRMS (ESI) calc'd for C₂₃H₁₉NOF₃ ([MH]⁺) 382.1413. Found: 382.1417.

### Example 4i

### General Procedure C for Palladium-Catalyzed Tandem Reactions—Synthesis of 2-(4-Fluoro-phenyl)-3-methyl-1-phenyl-1H-indole

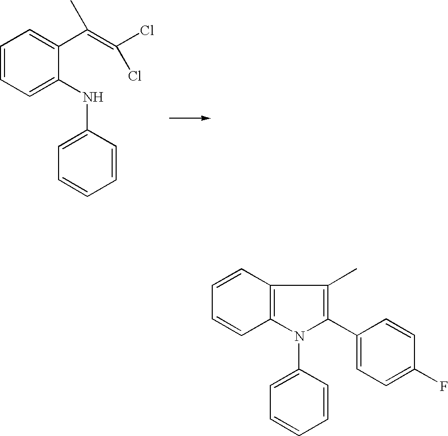

To a 5-mL round-bottom flask was charged with [2-(2,2-dichloro-1-methyl-vinyl)-phenyl]-phenyl-amine (0.056 g, 0.2 mmol), 4-FPhB(OH)₂ (0.042 g, 0.30 mmol), a powdered mixture of K₃PO₄.H₂O/KOH (mol/mol=1:2, 0.072 g, 0.6 mmol) and the mixture was purged with Ar for at least 10 min. To a separate 5-mL round-bottom flask was charged with Pd(OAc)₂ (1.34 mg, 3 mol %) and s-Phos (3.3 mg, 6 mol %) and purged with Ar for at least 10 min. Dry toluene (1 mL) was added to the pre-catalyst flask and the mixture was stirred at rt for 3 min. The homogenous pre-catalyst solution was then cannulated to the reactant flask and the heterogenous mixture was stirred at rt for 2 min and heated to 90° C. After stirred at 100° C. for 1 h, the mixture was cooled to rt and diluted with Et₂O (5 mL). After aqueous workup, the mixture was purified by flash chromatography (2.5% EtOAc in hexanes) to afford a white crystalline solid (0.058 g, 96%). R_{f}=0.22 (2.5% EtOAc in hexanes). mp 154-155° C. IR (neat, cm⁻¹) 3053 (w), 1995 (m), 1499 (s), 1452 (m), 1362 (w), 1217 (s). ¹H NMR (400 MHz, CDCl₃) δ 7.66-7.64 (1H, m), 7.35-7.26 (4H, m), 7.23-7.13 (6H, m), 6.96 (2H, ddd, J=7.7, 7.7, 2.0 Hz), 2.38 (3H, s). ¹³C NMR (100 MHz, CDCl₃) δ 162.1 (J_{CF}=248 Hz), 138.7, 137.8, 136.1, 132.4 (J_{CF}=7.7 Hz), 129.3, 129.1, 128.4 (J_{CF}=3.1 Hz), 128.1, 127.0, 122.8, 120.4, 119.1, 115.3 (J_{CF}=21.5 Hz), 110.9, 110.5, 9.7. ¹⁹F NMR (376 MHz, CDCl₃) δ −114.4 (1F, dddd, J_{FH}=8.6, 8.6, 5.8, 5.8 Hz). HRMS calc'd for C₂₁H₁₆NF ([M]⁺) 301.1267. Found: 301.1257.

### Example 4j

### Synthesis of 1-[4-(3-Methyl-2-o-tolyl-indol-1-yl)-phenyl]-ethanone

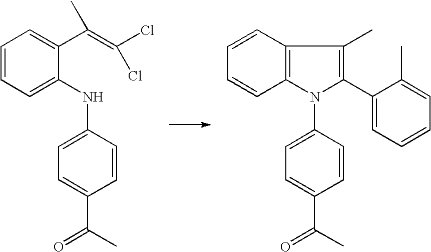

Following General Procedure C, a mixture of 1-{4-[2-(2,2-dichloro-1-methyl-vinyl)-phenylamino]-phenyl}-ethanone (0.096 g, 0.30 mmol), 2-MePhB(OH)₂ (0.061 g, 0.45 mmol), a powdered mixture of K₃PO₄.H₂O/KOH (mol/mol=1:2, 0.108 g, 0.9 mmol), and catalyst solution (Pd(OAc)₂ (2.2 mg, 3 mol %) and s-Phos (8.1 mg, 6 mol %) in PhMe (1.5 mL)) was heated at 100° C. for 3 h. After an aqueous workup, the crude was purified by flash chromatography on silica gel (10% EtOAc in hexanes) to afford a white solid (0.076 g, 75%). R_{f}=0.21 (10% EtOAc in hexanes). mp 138-139° C. IR (neat, cm⁻¹) 3055 (w), 2917 (w), 1683 (s), 1599 (s), 1455 (s), 1362 (s), 1266 (s). ¹H NMR (400 MHz, CDCl₃) δ 7.88-7.85 (2H, m), 7.68-7.64 (1H, m), 7.45-7.40 (1H, m), 7.26-7.20 (5H, m), 7.19-7.14 (3H, m), 2.55 (3H, s), 2.21 (3H, s), 2.00 (3H, s). ¹³C NMR (100 MHz, CDCl₃) δ 197.3, 142.9, 138.4, 136.8, 136.5, 134.7, 132.0, 131.7, 130.3, 129.5, 129.3, 128.7, 126.7, 125.7, 122.8, 120.8, 119.2, 112.3, 110.4, 26.7, 20.0, 9.5. HRMS (ESI) calc'd for C₂₄H₂₂NO ([MH]) 340.1695. Found: 340.1711.

### Example 4k

### Synthesis of 2-(3,4-Dimethoxy-phenyl)-1-phenyl-1H-indole

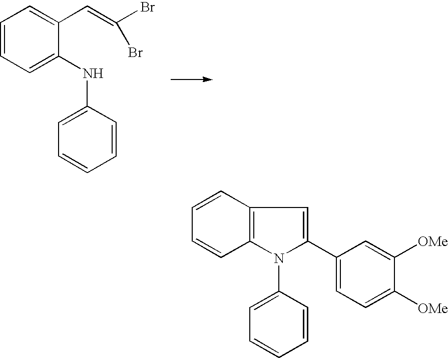

Following General Procedure C, a mixture of [2-(2,2-dibromo-vinyl)-phenyl]-phenyl-amine (0.106 g, 0.30 mmol), 3,4-(MeO)₂PhB(OH)₂ (0.082 g, 0.45 mmol), a powdered mixture of K₃PO₄.H₂O/KOH (mol/mol=1:2, 0.108 g, 0.9 mmol), and catalyst solution (Pd(OAc)₂ (2.2 mg, 3 mol %) and s-Phos (8.1 mg, 6 mol %) in PhMe (1.5 mL)) was heated at 100° C. for 5 h. After an aqueous workup, the crude was purified by flash chromatography on silica gel (20% EtOAc in hexanes) to afford a white solid (0.060 g, 60%). R_{f}=0.22 (20% EtOAc in hexanes). mp 113-115° C. IR (neat, cm⁻¹) 3057 (w), 2934 (w), 1596 (m), 1502 (s), 1454 (s), 1247 (s), 1224 (m), 1140 (m), 1025 (m). ¹H NMR (400 MHz, CDCl₃) δ 7.68-7.64 (1H, m), 7.44-7.40 (2H, m), 7.36-7.32 (1H, m), 7.28-7.25 (3H, m), 7.18-7.13 (2H, m), 6.94 (1H, dd, J=8.3, 2.0 Hz), 6.77 (1H, d, J=9.2 Hz), 6.76 (1H, s), 6.65 (1H, d, J=2.0 Hz), 3.85 (3H, s), 3.57 (3H, s). ¹³C NMR (100 MHz, CDCl₃) δ 148.6, 148.5, 140.8, 139.0, 138.9, 129.5, 128.5, 128.4, 127.4, 125.4, 122.3, 121.7, 120.9, 120.5, 112.4, 111.1, 110.7, 102.9, 56.0, 55.7. HRMS calc'd for C₂₂H₁₉NO₂ ([M]⁺) 329.1416. Found: 329.1424.

### Preparation of 3-[5-[[4-(methylsulfonyl)-1-piperazinyl]methyl]-1H-indole-2-yl]quinolin-2(1H)-one

The results of the preparation of the 3-[5-(4-Methanesulfonyl-piperazin-1-ylmethyl)-1H-indol-2-yl]-2-methoxy-quinoline precursor to 3-[5-[[4-(methylsulfonyl)-1-piperazinyl]methyl]-1H-indole-2-yl]quinolin-2(1H)-one are shown in Examples 5a-5e below.

### Example 5a

### 2-Methoxy-3-quinolin-3-ylboronic acid

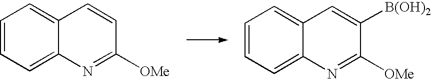

To a solution of to 2-methoxyquinoline (10.0 g, 62.8 mmol) and triisiopropylborate (17.86 g, 95.1 mmol) in THF (140 mL) at −78° C. was added LDA solution (75.4 mmol, prepared from Prⁱ₂NH and n-BuLi). The mixture was stirred at −78° C. for over 4 hours and slowly warmed to rt overnight. The mixture was quenched with saturated NH₄Cl (68 mL) and acidified to a pH=5 with 3M HCl. The organic solvent THF and hexanes were evaporated under vacuum and boronic acid was precipitated as a white solid. The mixture was filtered through a Buchner funnel and the solid was washed thoroughly with H₂O to afford the product after dried under high vacuum (12.11 g, 95%). ¹H NMR (400 MHz, CDCl₃) δ 8.64 (1H, s), 7.85 (1H, d, J=8.3 Hz), 7.80 (1H, J=7.9 Hz), 7.68 (1H, dd, J=14.1, 1.1 Hz), 7.41 (1H, J=7.5 Hz), 5.91 (2H, s, br), 4.18 (3H, s). ¹³C NMR (400 MHz, CDCl₃) δ 164.9, 149.8, 148.1, 131.1, 128.6, 127.4, 125.5, 124.7, 53.9. HRMS (EI) calc'd for C₁₀H₁₀BNO₃ ([M]⁺) 203.0754. Found: 203.0758.

### Example 5b

### 2-(2-Methoxy-quinolin-3-yl)-1H-indole-5-carboxylic acid methyl ester

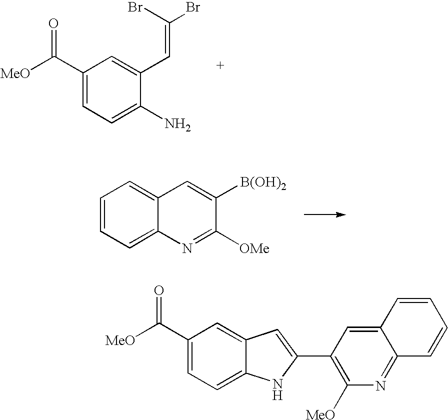

To a 5 mL round bottom flask was charged with 4-amino-3-(2,2-dibromo-vinyl)-benzoic acid methyl ester ((0.1675 g, 0.5 mmol), 2-methoxy-3-quinolin-3-ylboronic acid (0.1523 g, 0.75 mmol), Pd(OAc)₂ (3.4 mg, 0.015 mmol), S-Phos (12.3 mg, 0.03 mmol), and powdered K₃PO₄.H₂O (0.58 g, 2.5 mmol). The solid mixture was purged with argon for 10 min and toluence (2.5 mL) was added. The mixture was stirred at rt for 2 min and allowed to heated at 100° C. for 1.5 h. The mixture was diluted with EtOAc (10 mL) and H₂O and the organic phase was separated, dried over Na₂SO₄. The solid after removal of solvent was then chromatographed with 20% EtOAc/hexanes to afford a white product (0.143 g, 86%). ¹H NMR (300 MHz, DMSO) δ 11.89 (1H, s), 8.74 (1H, s), 8.31 (1H, s), 7.94 (1H, d, J=7.2 Hz), 7.84-7.77 (2H, m), 7.70 (1H, dd, J=7.0, 1.3 Hz), 7.56 (1H, d, J=8.5 Hz), 7.50 (1H, dd, J=6.9, 1.2 Hz), 7.32 (1H, d, J=1.3 Hz), 4.18 (3H, s), 3.86 (3H, s). ¹³C NMR (100 MHz, DMSO) δ 167.2, 158.3, 144.7, 139.4, 135.5, 134.2, 130.0, 127.8, 127.7, 126.4, 124.9, 124.8, 123.0, 122.9, 120.9, 116.5, 111.4, 104.7, 53.8, 51.7. HRMS calc'd for C₂₀H₁₆N₂O₃ ([M]⁺) 332.1161. Found: 332.1161.

### Example 5c

### [2-(2-Methoxy-quinolin-3-yl)-1H-indol-5-yl]-methanol

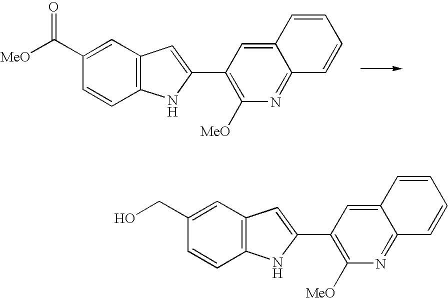

To a suspension of the methyl ester (0.543 g, 1.63 mmol) in dry Et₂O (15 mL) at −15° C. was added LiAlH₄ (0.312 g, 8.2 mmol) in two portions under argon. The mixture was vigorously stirred at under 0° C. for 5 h and then was quenched with NH₄Cl (10 mL). The mixture was extracted with sufficient amount of EtOAc until not product was observed in the aqueous phase. The solution was washed with brine and dried over Na₂SO₄. The residue after removal of solvent was chromatographed with 1:1 EtOAc/hexanes to afford the product as slightly yellow solid (0.471 g, 95%). ¹H NMR (400 MHz, CDCl₃) δ 9.66 (1H, s, br), 8.43 (1H, s), 8.43 (1H, s), 7.85 (1H, d, J=8.3 Hz), 7.76 (1H, dd, J=7.9, 1.3 Hz), 7.63-7.59 (2H, m), 7.44-7.39 (2H, m), 7.22 (1H, dd, J=8.3, 1.5 Hz), 7.04 (1H, dd, J=2.2, 0.9 Hz), 4.79 (2H, d, J=5.7 Hz), 4.31 (3H, s), 1.58 (1H, t, J=5.7 Hz). ¹³C NMR (100 MHz, DMSO) δ 158.3, 145.4, 136.2, 135.4, 134.3, 133.1, 129.8, 128.4, 127.7, 127.2, 125.7, 125.0, 122.7, 119.5, 116.7, 111.6, 101.4, 66.5, 54.2. ESI-HRMS calc'd for C₁₉H₁₇N₂O₂ ([MH]⁺) 305.1284. Found: 305.1281.

### Example 5d

### 2-(2-Methoxy-quinolin-3-yl)-1H-indole-5-carbaldehyde

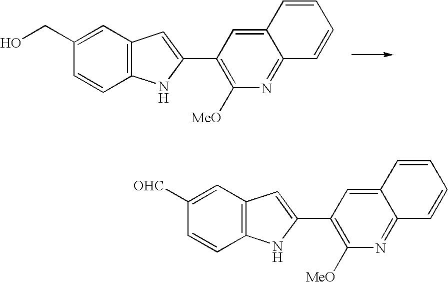

To a mixture of the alcohol (0.266 g, 0.874 mmol), 4-methylmorpholine N-oxide (NMO) (0.151 g, 1.31 mmol), and 4 Å molecular sieves (0.3 g) was added dry DCM (8.5 mL) and the mixture was stirred at rt for 10 min before addition of tetrapropylammonium perruthenate (TPAP) (6.1 mg, 0.00175 mmol). The reaction mixture was stirred at rt for 24 h before quenched by addition of Na₂SO₃ (10 mL) and diluted with HOAc (20 mL). Organic phase was separated and washed with brine and dried over Na₂SO₄. The residue after removal of solvent was chromatographed with 25% EtOAc/hexanes to afford a slightly yellow solid (0.241 g, 91%). ¹H NMR (400 MHz, CDCl₃) δ 10.06 (1H, s), 9.96 (1H, br), 8.52 (1H, s), 8.20 (1H, d, J=0.7 Hz), 7.88 (1H, d, J=8.3 Hz), 7.83-7.79 (2H, m), 7.66 (1H, ddd, J=7.9, 7.0, 1.5 Hz), 7.55 (1H, d, J=8.1 Hz), 7.45 (1H, ddd, J=7.9, 7.9, 1.5 Hz), 7.22 (1H, dd, J=2.2, 0.9 Hz), 4.32 (3H, s). ¹³C NMR (75 MHz, CDCl₃) δ 192.7, 158.1, 145.7, 139.9, 136.0, 135.8, 130.3, 130.2, 128.1, 127.8, 127.2, 125.9, 125.5, 125.2, 123.0, 115.9, 112.0, 102.7, 54.3. ESI-HRMS calc'd for C₁₉H₁₅N₂O₂ ([MH]⁺) 303.1128. Found: 303.1130.

### Example 5e

### 3-[5-(4-Methanesulfonyl-piperazin-1-ylmethyl)-1H-indol-2-yl]-2-methoxy-quinoline

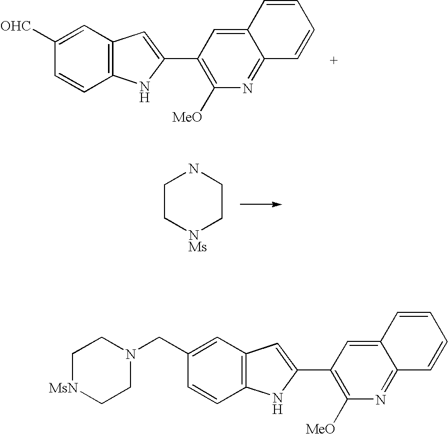

To a mixture of the aldehyde (75.6 mg, 0.248 mmol), the amine (41 mg, 0.25 mmol), and 4 Å molecular sieves (0.1 g) was added dry DCM (5 mL). The mixture was added NaHB(OAc)₃ (79.5 mg, 0.375 mmol) and the mixture was stirred at rt for 24 h. The mixture was filtered through a celite pad and washed with copious amount of EtOAc. The residue after removal of solvent was chromatographed with 100% EtOAc to afford a slightly yellow solid (0.1035 g, 93%). ¹H NMR (400 MHz, CDCl₃) δ 9.65 (1H, s), 8.43 (1H, s), 7.86 (1H, d, J=8.3 Hz), 7.77 (1H, d, J=7.9 Hz), 7.61 (1H, t, J=7.6 Hz), 7.54 (1H, s), 7.43-7.40 (2H, m), 7.17 (1H, d, J=8.3 Hz), 7.03 (1H, s), 4.27 (3H, s), 3.64 (2H, s), 3.24 (4H, br), 2.75 (3H, s), 2.59 (4H, br). ¹³C NMR (75 MHz, CDCl₃) δ 158.4, 145.5, 136.0, 135.4, 134.2, 129.8, 129.2, 128.3, 127.6, 127.2, 125.6, 125.0, 124.3, 121.2, 116.8, 111.3, 101.3, 63.4, 54.2, 52.4, 46.2, 34.2. ESI-HRMS calc'd for C₂₄H₂₇N₄O₃S ([MH]⁺) 451.1798. Found: 451.1799.

Although the invention has been shown and described with respect to illustrative embodiments thereof, it should be appreciated that the foregoing and various other changes, omissions and additions in the form and detail thereof may be made without departing from the spirit and scope of the invention as delineated in the claims.

1. (canceled)

2. (canceled)

3. A process for the preparation of a 2-substituted indole compound of formula (IV) 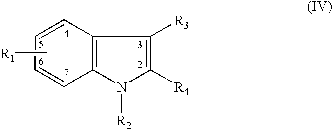
wherein
each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the indole ring; all of which are optionally substituted with one or more suitable substituents at one or more substitutable positions,
R₂ comprises H, alkyl, cycloalkyl, aryl, heteroaryl, aryl-loweralkyl-, or heteroaryl-loweralkyl-, all of which are optionally substituted at one or more substitutable positions with one or more suitable substituents,
R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents, and
R₄ is selected from the group consisting of monocyclic aromatic, polycyclic aromatic, monocyclic heteroaromatic, polycyclic heteroaromatic, 1° alkyl, and alkenyl, all of which are optionally substituted at one or more substitutable positions with one or more suitable substituents, and wherein R₄ is bonded to the 2-position of the indole ring via a C—C bond;
the process comprising reacting an ortho-gem-dihalovinylaniline compound of formula (V) 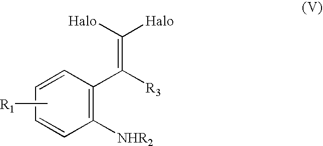
wherein
R₁, R₂ and R₃ are as defined above, and Halo comprises bromo, chloro, or iodo;
with an organoboron reagent selected from the group consisting of a boronic ester of R₄, a boronic acid of R₄, a boronic acid anhydride of R₄, a trialkylborane of R₄ and a 9-BBN derivative of R₄;
in the presence of a base, a palladium metal pre-catalyst and a ligand under reaction conditions effective to form the 2-substituted indole compound.

4.-13. (canceled)

14. The process of claim 3, wherein the organoboron reagent comprises a boronic acid of R₄.

15. The process of claim 3, wherein the organoboron reagent comprises a 9-BBN derivative of R₄.

16. The process of claim 3, wherein the organoboron reagent comprises a trialkylborane of R₄.

17. The process of claim 3, wherein the pre-catalyst comprises Pd(OAc)₂, Pd(PPh₃)₄, Pd₂(dba)₃, Pd(CH₃CN)₂Cl₂, PdCl₂, K₂PdCl₄, or Pd₂(dba)₃.CHCl₃.

18. The process of claim 17, wherein the pre-catalyst comprises Pd(OAc)₂ and the organoboron reagent comprises a boronic acid of R₄.

19. The process of claim 17, wherein the pre-catalyst comprises Pd₂(dba)₃, and the organoboron reagent comprises a 9-BBN derivative of R₄.

20. (canceled)

21. (canceled)

22. The process of claim 3, wherein the ligand comprises a phosphorous-containing ligand or a nitrogen-containing carbenoid ligand.

23. (canceled)

24. (canceled)

25. The process of claim 22, wherein the ligand comprises s-Phos, P(o-tol)₃, PPh₃, P(O—CF₃-Ph)₃, BINAP, tol-BINAP, dppm, dppe, dppp, dppb, dppf, Xanphos, BIPHEP, AsPh₃, or

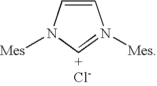

26.-28. (canceled)

29. The process of claim 3, wherein the base comprises an organic base or an inorganic base.

30. The process of claim 29, wherein the base comprises a metal carbonate, a metal hydroxide, a metal phosphonate, or a trialkylamine.

31. The process of claim 30, wherein the base comprises K₂CO₃, Na₂CO₃, Cs₂CO₃, NaOH, K₃PO₄, K₃PO₄.H₂O, or NEt₃.

32. (canceled)

33. The process of claim 32, wherein the ligand comprises s-Phos, the base comprises K₃PO₄.H₂O, and the catalyst comprises Pd(OAc)₂.

34.-42. (canceled)

43. The process of claim 3 wherein the ortho-gem-dihalogen vinylaniline compound of formula (V) 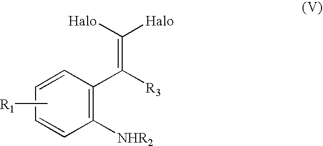
wherein each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₃ is H, CF₃, or alkynyl optionally substituted at one or more positions with one or more suitable substituents, R₂ is H, and Halo comprises bromo, is prepared by a process comprising the steps of:
(a) reacting a nitrobenzaldehyde or ketone compound of formula (VI) 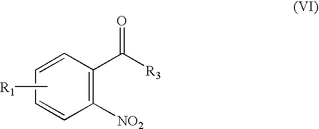
wherein R₁ is as defined above, and R₃ is as defined above, with CBr₄ and PPh₃ under conditions effective to generate in situ the ortho-gem-dihalovinyl compound of formula (VII) 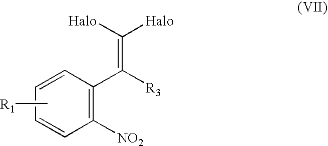
wherein R₁ is as defined above, R₃ is as defined above, and Halo is bromo; and
(b) reducing the compound of formula (VII) under conditions effective to reduce the nitro group of the compound of formula (VII) without affecting the functional groups present in the compound, to afford the compound of formula (V).

44.-50. (canceled)

51. The process of claim 3 wherein the ortho-gem-dihalovinylaniline compound of formula (V) 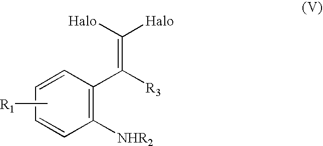
wherein each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₂ is H and R₃ is H, alkyl, or alkynyl optionally substituted at one or more positions with one or more suitable substituents, and Halo comprises chloro, is prepared by a process comprising the steps of:
(a) reacting a nitrobenzaldehyde or ketone compound of formula (VI) 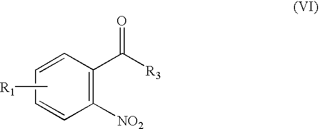
wherein R₁ and R₃ are as defined above, with 2 or more equivalents of CHCl₃ and PPh₃ in the presence of 2 or more equivalents of KO^{t}Bu, wherein said equivalents are relative to formula (VI), under conditions effective to generate in situ the ortho-gem-dichlorovinyl compound of formula (VII) 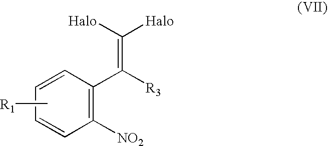
wherein R₁ and R₃ are as defined above and Halo is chloro; and
(b) reducing the compound of formula (VII) under conditions effective to reduce the nitro group of the compound of formula (VII), without affecting the functional groups present in the compound, to afford the compound of formula (V).

52.-56. (canceled)

57. The process of claim 3 wherein the ortho-gem-dihalovinylaniline compound of formula (V) 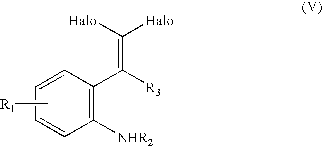
wherein each of the one or more R₁ substituents is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (V); all of which are optionally substituted with one or more suitable substituents at one or more substitutable positions; R₂ comprises H; R₃ comprises alkyl, haloalkyl, alkenyl, alkynyl, aryl, heteroaryl, cycloalkyl, heterocycle, aryl-loweralky-, or heteroaryl-loweralkyl-, all of which are optionally substituted at one or more substitutable positions with one or more suitable substituents; and Halo comprises bromo or chloro, is prepared by a process comprising the steps of:
(a) converting a ketone of formula (VIII) 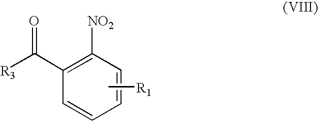
wherein R₁ and R₃ are as defined above into the corresponding olefin derivative of formula (IX) under conditions effective to generate the corresponding olefin derivative of formula (IX) 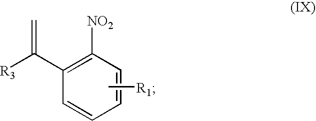
(b) halogenating the olefin derivative of formula (IX) under conditions effective to generate the diahalogen compound of formula (X) 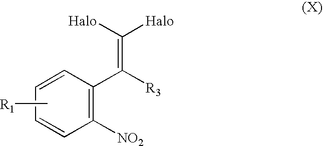
wherein R₁, Halo, and R₃ are defined above; and
(c) reducing the compound of formula (X) under conditions effective to reduce the nitro group of the compound of formula (X) without affecting the functional groups present in the compound, to afford the compound of formula (V).

58.-65. (canceled)

66. The process of claim 3 wherein the compound of Formula V comprises an N-arylaniline compound of formula (XI) 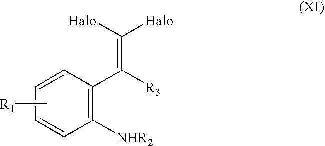
wherein Halo comprises Br, Cl, or I; R₂ comprises aryl which is optionally substituted at one or more substitutable positions with one or more suitable substituents; R₃ comprises H, alkyl, haloalkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (XI); all of which are optionally substituted with one or more suitable substituents at one or more substitutable positions; said N-arylaniline compound of formula (XI) being prepared by a process comprising the steps of:
(a) reacting a compound of formula (V) 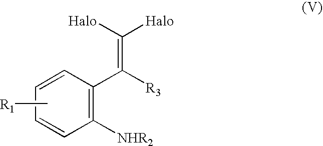
wherein Halo, R₁, R₃ are as defined in Formula (XI) above and R₂ is H, with an organoboron reagent comprising a boronic acid, boronic acid anhydride or BF₃⁻ salt of R₂ in the presence of at least about 1 equivalent of a copper (II) catalyst relative to the compound of formula (V), at least about 0.3 equivalents of a C₈-C₂₀ fatty acid relative to the compound of formula (V), molecular oxygen, and a non-nucleophlic base, at a reaction temperature of between about 40° C. and 60° C., under conditions effective to form a C—N bond between formula (V) and the R₂ group of the organoboron reagent, to afford the N-arylaniline compounds of formula (XI).

67.-69. (canceled)

70. The process of claim 3, wherein each of the one or suitable substituents at the one or more substitutable positions is independently selected from the group consisting of H; hydroxyl; cyano; alkyl; alkoxy; aryloxy; vinyl; alkenyl; alkynyl; formyl; haloalkyl; halogen; aryl; heteroaryl; amido; acyl; ester; ether; thioether; amino; thioalkoxy; and phosphino.

71. (canceled)

72. A process for the preparation of fluvastatin 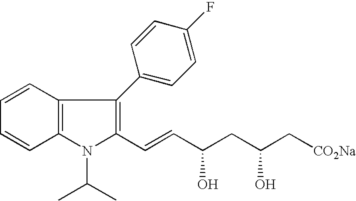
comprising the steps of:
(a) reacting 2,2-dibromo-1-(4-fluorophenyl)-1-(2-aminophenyl)ethene: 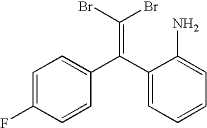
under conditions effective to prepare {2-[2,2-Dibromo-1-(4-fluoro-phenyl)-vinyl]-phenyl} isopropylamine: 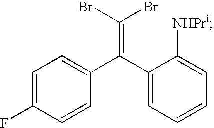
(b) coupling {2-[2,2-Dibromo-1-(4-fluoro-phenyl)-vinyl]-phenyl}isopropylamine with a boronic acid fragment of the formula: 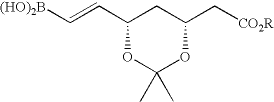
wherein R comprises methyl, ethyl, or t-butyl, under conditions effective to form an indole of the formula (6-{2-[3-(4-fluorophenyl)-1-isoproyl-1H-indole-2-yl]-vinyl}-2,2-dimethyl-[1,3]dioxan-4-yl)acetic acid alkyl esters: 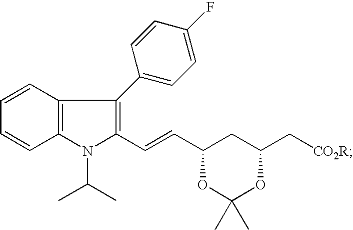
(c) reacting said indole under conditions effective to generate a lactone of the formula 6-{2-[3-(4-fluorophenyl)-1-isoproyl-1H-indole-2-yl]-vinyl}-4-hydroxytetrahydropyran-2-one: 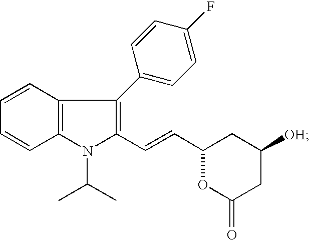
(d) reacting said lactone under conditions effective to generate fluvastatin.

73.-85. (canceled)

86. A process for the preparation of 3-[5-[[4-(methylsulfonyl)-1-piperazinyl]methyl]-1H-indole-2-yl]quinolin-2(1H)-one: 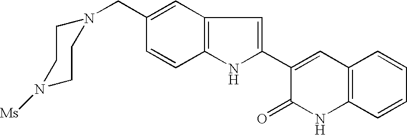
comprising the steps of:
(a) coupling 4-amino-3-(2,2-dibromo-vinyl)-benzoic acid methyl ester: 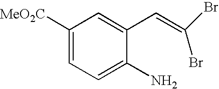
with 2-methoxyquinolinylboronic acid 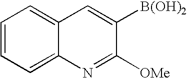
under conditions effective to form 2-(2-methoxy-quinolin-3-yl)-1H-indole-5-carboxylic acid methyl ester: 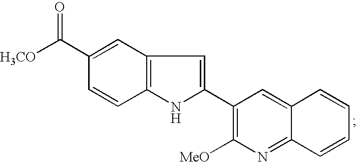
(b) reducing 2-(2-methoxy-quinolin-3-yl)-1H-indole-5-carboxylic acid methyl ester under conditions effective to form [2-(2-Methoxy-quinolin-3-yl)-1H-indol-5-yl]-methanol: 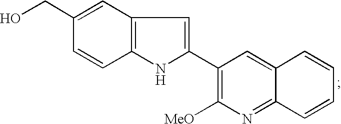
(c) converting [2-(2-Methoxy-quinolin-3-yl)-1H-indol-5-yl]-methanol to 2-(2-Methoxy-quinolin-3-yl)-1H-indole-5-carbaldehyde: 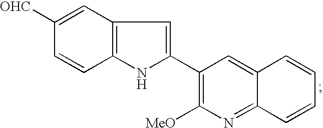
(d) coupling 2-(2-Methoxy-quinolin-3-yl)-1H-indole-5-carbaldehyde with N-methanesulfonyl piperazine to yield 3-[5-(4-Methanesulfonyl-piperazin-1-ylmethyl)-1H-indol-2-yl]-2-methoxy-quinoline: 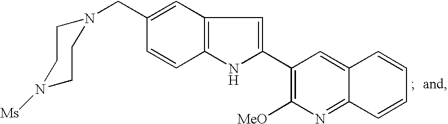
(e) converting 3-[5-(4-Methanesulfonyl-piperazin-1-ylmethyl)-1H-indol-2-yl]-2-methoxy-quinoline to 3-[5-[[4-(methylsulfonyl)-1-piperazinyl]methyl]-1H-indole-2-yl]quinolin-2(1H)-one.

87.-93. (canceled)

94. A The process of claim 3 wherein the compound of Formula (V) comprises an N-alkylaniline compound of formula (XI) 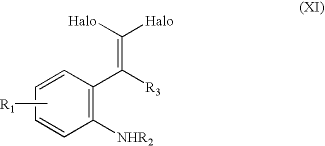
wherein Halo comprises Br, Cl, or I; R₂ comprises alkyl which is optionally substituted at one or more substitutable positions with one or more suitable substituents; R₃ comprises H, alkyl, alkenyl, alkynyl, aryl, heteroaryl, cycloalkyl, heterocycle, aryl-loweralkyl-, or heteroaryl-loweralkyl-, all of which are optionally substituted at one or more substitutable positions with one or more suitable substituents; and each of the one or more R₁ is independently selected from the group consisting of H, fluoro, lower alkyl, lower alkenyl, lower alkoxy, aryloxy, lower haloalkyl, lower alkenyl, —C(O)O-lower alkyl, monocyclic or polycyclic aryl or heteroaryl moiety, or R₁ is an alkenyl group bonded so to as to form a 4- to 20-membered fused monocycle or polycyclic ring with the phenyl ring of Formula (XI); all of which are optionally substituted with one or more suitable substituents at one or more substitutable positions; said N-alkylaniline compound of formula (XI) being prepared by a process comprising:
reacting a compound of formula (V) 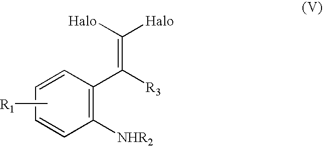
wherein Halo, R₁, R₃ are as defined in Formula (XI) above and R₂ is H, with a suitable alkylating agent under conditions effective to form a C—N bond between formula (V) and the alkyl group of the alkyl halide, to afford the N-alkylaniline compounds of formula (XI).

95.-96. (canceled)
